(19) United States
(12) Patent Application Publication
(10) Pub. No.: US 2014/0018384 A1
(43) Pub. Date: Jan. 16, 2014

(54) QUINOLINONE DERIVATIVES FOR USE IN THE TREATMENT OF AN AUTOIMMUNE DISEASE AND/OR AN INFLAMMATORY DISEASE

(71) Applicants: Karl Fredrik Wallner, Goteborg (SE); Jan Peter Olofsson, Sjuntorp (SE); Malin Kristin Hultqvist, Harlosa (SE); Benjamin Harry Pelcman, Stockholm (SE)

(72) Inventors: Karl Fredrik Wallner, Goteborg (SE); Jan Peter Olofsson, Sjuntorp (SE); Malin Kristin Hultqvist, Harlosa (SE); Benjamin Harry Pelcman, Stockholm (SE)

(73) Assignee: PRONOXIS AB, Lund (SE)

(21) Appl. No.: 14/005,773

(22) PCT Filed: Mar. 16, 2012
(86) PCT No.: PCT/GB2012/050586

§ 371 Date: Sep. 17, 2013

(30) Foreign Application Priority Data
Mar. 18, 2011 (GB) 1104600.0

(51) Int. Cl. A61K 31/4741; C07D 215/233; G01N 33/50; C07D 471/04; A61K 31/4375; A61K 45/06; C07D 491/04; A61K 31/47

(52) U.S. Cl. CPC A61K 31/4741; C07D 491/04; C07D 215/233; A61K 31/47; C07D 471/04; A61K 31/4375; A61K 45/06; G01N 33/5038
USPC 514/291; 546/90; 546/156; 514/312; 546/123; 514/300; 435/25

(57) ABSTRACT

There is provided compounds of formula I, wherein X¹ to X⁴, R¹ to R⁴, Y¹, Y² and L are as defined in the description, and pharmaceutically-acceptable salts thereof, which may be useful in the treatment and/or prophylaxis of autoimmune diseases, inflammatory (e.g. chronic inflammatory) diseases and/or other diseases that may benefit from production of ROS (reactive oxygen species) by a NADPH oxidase complex.

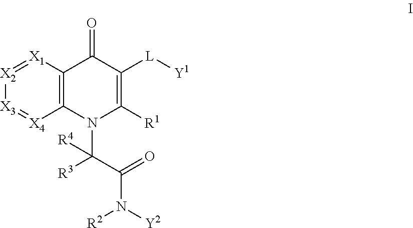

### FIELD OF THE INVENTION

The invention relates to novel compounds, compositions and methods for treatment, prevention and/or prophylaxis of autoimmune diseases and inflammatory (e.g. chronic inflammatory) conditions. In particular, the invention relates to novel compounds, compositions and methods for the treatment and/or prophylaxis of common autoimmune diseases including rheumatoid arthritis (RA) and variants thereof, multiple sclerosis (MS) and inflammatory bowel disease (IBD) through stimulation of production of reactive oxygen species (ROS) by a NADPH oxidase complex.

### BACKGROUND OF THE INVENTION

The listing or discussion of an apparently prior-published document in this specification should not necessarily be taken as an acknowledgement that the document is part of the state of the art or is common general knowledge.

### Autoimmune Diseases

Autoimmune diseases and hyperinflammatory disorders are conditions where a mammal's immune system starts reacting against its own tissues. Such conditions include, without limitation, arthritis, e.g. rheumatoid arthritis (RA), multiple sclerosis (MS), inflammatory bowels disease (IBD), systemic lupus erythematosus (SLE), autoimmune uveitis, type I diabetes, dermatomyesitis, Goodpasteure's syndrome, Graves' disease, Guillian-Barré Syndrome (GBS), Hashimotos Disease, Mixed connective tissue disease, Myasthenia gravis, Pemphigus vulgaris, Pernicious anemia, Psoriasis, Polymyositis Primary biliary cirrhosis, Sjögren's syndrome, Giant cell arteritis, Ulcerative colitis, Vasculitis, Wegener's granulomatosis, Churg-Strauss syndrome, postpolio syndrome and idiopathic thrombocytopenic purpura.

Shortcomings and Complications with Current Treatment

Today's treatment of autoimmune conditions like RA is not effective for all patients with diagnosed disease also including a large proportion of patients that experience adverse effects from treatments with biological agents, as represented by the therapy with TNF-α inhibitors, or from treatment with Methotrexate and COX-2 inhibitors. In similarity to RA, the cause and pathology of autoimmune and (hyper) inflammatory conditions including MS, IBD and the majority of less prevalent autoimmune conditions, is far from understood and many patients suffer from a disease that current treatments do not have the capacity to treat or ameliorate, hence there is a great need to understand the mechanisms driving these diseases which will enable novel ways for treatments.

The present invention aims at providing new treatments for inflammatory and autoimmune diseases based on immunomodulatory effects that can be achieved by stimulation of ROS (reactive oxygen species) production by a NADPH oxidase complex.

The role of ROS in immunity is complex and both anti-inflammatory and pro-inflammatory responses to ROS have been reported. Traditionally, ROS is considered harmful and promoting inflammatory responses. However, CGD (Chronic granulomateous disease) caused by defects in any of the subunits of a NADPH oxidase complex may be characterized not only by inability to produce ROS resulting in defective bactericidal and fungiciadal capacity, but is also associated with hyper-inflammatory syndromes similar to idiopatic inflammatory diseases (Bjorkman, Dahlgren et al. 2008), suggesting an inflammation dampening effect of ROS. CGD patients also have a higher risk of developing autoimmune diseases (De Ravin, Naumann et al. 2008). Direct evidence for involvement of ROS in autoimmunity has also been reported, since a correlation between defect capacity to produce ROS and increased severity of autoimmune inflammation has been shown in indications like Guillian Barré Syndrome (Mossberg, Andersen et al. 2007) and MS (Mossberg, Movitz et al. 2009).

The idea to activate ROS production as a way to treat inflammatory conditions was postulated when the organising component, P47^{PHOX} (NCF1), of a NADPH oxidase complex was found to regulate autoimmunity in a rat model of RA. The importance of a functional NADPH oxidase complex was verified both in rat and mouse models of arthritis (Olofsson, Holmberg et al. 2003) (Hultqvist, Olofsson et al. 2004) where animals with genetic variations of Ncf1 causing low ROS production was rendered more susceptible to development of disease. The role of ROS in autoimmune models has also been suggested as a potential novel way for treatment of RA (Hultqvist, Olofsson et al. 2006).

To further strengthen the importance of functional ROS production and correlation with autoimmunity an association between polymorphisms in one of the genes in a NADPH oxidase complex, i.e. NCF4 (encoding the P40^{PHOX} subunit) and RA in rheumatoid factor negative men has been confirmed (Olsson, Lindqvist et al. 2007) as well as an association with NCF4 in Crohn's disease (Rioux, Xavier et al. 2007; Roberts, Hollis-Moffatt et al. 2008). In addition, NCF2 (encoding p67^{PHOX}) was found to be associated with arthritis in Chinese SLE patients (Yu, Chen et al.). These findings together argue for a general ROS-regulated pathway affecting autoimmunity. Similarly, a recent study proposed a role of number of copies (functional versus pseudo) of NCF1, determining responses to infections and autoimmunity (Greve, Hoffmann et al. 2008).

Use of inducers of ROS from a NADPH oxidase complex has been described in WO 2008/028002, WO 2007/104790, WO 2003/095667, US 2008/0227135, US 2004/0009901 all of which are hereby incorporated by reference.

Small molecules that activate ROS production have been selected and produced through screening processes (Schepetkin, Kirpotina et al. 2007; Schepetkin, Kirpotina et al. 2008). (Kirpotina, Khlebnikov et al.)(Forsman, Kalderen et al.), However, most attempts to modulate the effect of a NADPH oxidase are directed to the identification of compounds that inhibit ROS production (Lambeth, Krause et al. 2008; Jaquet, Scapozza et al. 2009). For most of such screens it is not known the direct activating mechanism for induction of ROS production (Bae, Lee et al. 2003; Krjukova, Holmqvist et al. 2004). The present invention is aimed at treatment of autoimmune and other inflammatory (e.g. chronic inflammatory) conditions with small molecules that stimulate production of ROS from a NADPH oxidase complex.

International patent application WO 2009/086303 and US patent application US 2009/0163545 both disclose a method for altering the lifespan of various eukaryotic organisms and screening for such compounds. This document does not concern the increase of ROS production and the treatment of autoimmune diseases.

International patent application WO 2007/059108 discloses bicyclic compounds, including quinolinones and variants, which may act as antagonists to the chemokine receptor (and may therefore be useful in the treatment of e.g. cancer). However, this document only discloses compounds in which the quinolinone (or variant) is substituted on the nitrogen atom with an alkylene linker group. Further, this document does not relate to compounds that increase ROS production and therefore useful to treat e.g. autoimmune diseases.

International patent application WO 2008/107436 discloses various compounds including quinolinones (or variants), which may be useful as antagonists to sphingosine-1-phosphate receptors. However, this document does not disclose compounds in which the nitrogen atom of the quinolinone is substituted with an alkylamido linker group and the bicyclic ring is itself directly substituted with a heteroatom. Further, this document does not relate to compounds that increase ROS production and therefore useful to treat e.g. autoimmune diseases.

International patent application WO 2007/005779 discloses compounds of potential use as protease inhibitors. International patent application WO 2008/112715 discloses various compounds for potential use as calcium ion channel inhibitors. Neither of these documents discloses quinolinones (or variants) in which the nitrogen atom of the quinolinone is substituted with an alkylamido linker group. Nor do these documents relate to autoimmune diseases.

### DISCLOSURE OF THE INVENTION

There is provided a compound of formula I,

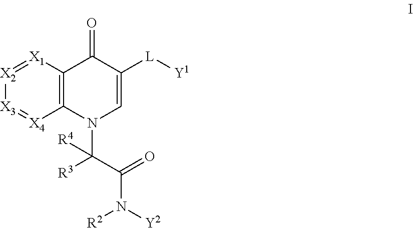

wherein:
X¹, X², X³ and X⁴ each represents CR⁵, CR⁶, CR⁷, CR⁸ respectively or, any one or two of X¹ to X⁴, alternatively and independently represents N;
R⁵, R⁶, R⁷ and R⁸ each independently represents hydrogen, halo, —R^{a}, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, —S(O)₂N(R^{r})R^{s}, or —SR^{t}; or
any two R⁵, R⁶, R⁷ or R⁸ groups, when adjacent to one another, may be linked by a —C₁₋₅alkylene-, —O—C₁₋₄alkylene-, —C₁₋₄alkylene-O—, —O—C₁₋₃alkylene-O—, —S—C₁₋₄alkylene-, —C₁₋₄alkylene-S— or —S—C₁₋₃alkylene-S— group, which alkylene moieties are optionally substituted by one or more substituent selected from —F, C₁₋₃alkyl (optionally substituted by one or more —F) and ═O;
but wherein at least one of R⁵ to R⁸ is present and represents —ORⁿ, —SR^{t} or halo, or two adjacent groups selected from R⁵ to R⁸ are both present and are linked together to form a —O—C₁₋₄alkylene-, —C₁₋₄alkylene-O—, —O—C₁₋₃alkylene-O—, —S—C₁₋₄alkylene-, —C₁₋₄alkylene-S— or —S—C₁₋₃alkylene-S— group;
L represents —C(O)— or —S(O)₂—;
Y¹ represents aryl or heteroaryl, both of which are optionally substituted by one or more substituents selected from E¹;
Y² represents aryl or heteroaryl, both of which are optionally substituted by one or more substituents selected from E²;
E¹ and E² each independently represent halo, —R^{a}, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, —S(O)₂N(R^{r})R^{s}, or —SR^{t}; and/or
any two E¹ or E² substituents, when adjacent to one another, may be linked to form a —C₁₋₆alkylene-, —O—C₁₋₄alkylene-, —O—C₁₋₃alkylene-O—, —S—C₁₋₄alkylene-, —C₁₋₄alkylene-S— or —S—C₁₋₃alkylene-S— group, which alkylene moieties are optionally substituted by one or more substituents selected from —F, C₁₋₃alkyl (optionally substituted by one or more —F) and ═O;
R¹ and R² each independently represent hydrogen or C₁₋₆ alkyl (optionally substituted by one or more substituents selected from —F, —OMe, —OEt, —OCHF₂ and —OCF₃);
R³ and R⁴ each independently represent hydrogen or C₁₋₆ alkyl (optionally substituted by one or more substituents selected from —F, —OMe, —OEt, —OCHF₂ and —OCF₃); or
R³ and R⁴ may be linked to form a further 3- to 6-membered ring, which may optionally contain one or more heteroatoms (e.g. one or two heteroatoms, such as one oxygen heteroatom) and may optionally be further substituted by one or more substituents selected from C₁₋₆alkyl (optionally substituted by one or more —F) and ═O;
each R^{a}, R^{b}, R^{c}, R^{d}, R^{e}, R^{f}, R^{g}, R^{h}, Rⁱ, R^{j}, R^{l}, Rⁿ, R^{o}, R^{q}, R^{r}, R^{s} and R^{t} independently represents hydrogen or C₁₋₆ alkyl (optionally substituted by one or more —F); or
any two R^{c} and R^{d}, R^{f} and R^{g} and/or R^{r} and R^{s} may be linked together with the nitrogen atom to which they are necessarily attached, to form a 3- to 8-membered ring (e.g. a 5- or 6-membered ring), such as a monocyclic or bicyclic ring, which ring may optionally contain one or two heteroatoms (e.g. one or two heteroatoms, such as one oxygen heteroatom), and which ring may optionally be substituted by one or more substituents selected from —F, C₁₋₃alkyl (optionally substituted by one or more —F) and ═O;
each R^{k}, R^{m} and R^{p} independently represent C₁₋₆ alkyl (optionally substituted by one or more —F);
m represents 0, 1 or 2,
or a pharmaceutically acceptable salt thereof,
for use in the treatment, prevention and/or prophylaxis of autoimmune diseases and chronic inflammatory diseases,
which compounds may be referred to herein as “the compounds of the invention”.

There also provided a compound of formula Ia, or a pharmaceutically acceptable salt thereof, for use in the treatment, prevention and/or prophylaxis of autoimmune diseases and chronic inflammatory diseases,

wherein the compound is as defined by formula I except that:

R⁵, R⁶, R⁷ and R⁸ each independently represents hydrogen, halo, —R^{a}, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, —S(O)₂N(R^{r})R^{s}, or —SR^{t}; or

any two R⁵, R⁶, R⁷ or R⁸ groups, when adjacent to one another, may be linked by a —C₁₋₅alkylene-, —O—C₁₋₄alkylene-, —C₁₋₄alkylene-O—, —O—C₁₋₃alkylene-O—, —S—C₁₋₄alkylene-, —C₁₋₄alkylene-S— or —S—C₁₋₃alkylene-S— group, which alkylene moieties are optionally substituted by one or more substituent selected from —F, C₁₋₃alkyl (optionally substituted by one or more —F) and ═O;
but wherein at least one of R⁶ and R⁷ is present and represents —ORⁿ or —SR^{t}, or R⁶ and R⁷ are both present and are linked together to form a —O—C₁₋₄alkylene-, —C₁₋₄alkylene-O≠, —O—C₁₋₄alkylene-O≠, —S—C₁₋₄alkylene-, —C₁₋₄alkylene-S— or —S—C₁₋₃alkylene-S— group.

In a certain embodiment that may be mentioned, there also provided a compound of formula I, or a pharmaceutically acceptable salt thereof, for use in the treatment, prevention and/or prophylaxis of autoimmune diseases and chronic inflammatory diseases, wherein:

R⁵, R⁶, R⁷ and R⁶ each independently represents hydrogen, halo, —R^{a}, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, —S(O)₂N(R^{r})R^{s}, or —SR^{t}; or
any two R⁵, R⁶, R⁷ or R⁸ groups, when adjacent to one another, may be linked by a —C₁₋₅alkylene-, —O—C₁₋₄alkylene-, —C₁₋₄alkylene-O≠, —O—C₁₋₃alkylene-O—, —S—C₁₋₄alkylene-, —C₁₋₄alkylene-S— or —S—C₁₋₃alkylene-S— group, which alkylene moieties are optionally substituted by one or more substituent selected from —F, C₁₋₃alkyl (optionally substituted by one or more —F) and ═O;
but wherein at least one of R⁵ and R⁸ is present and represents —ORⁿ—SR^{t} or halo, or any two adjacent groups selected from R⁵ and R⁶ and R⁷ and R⁸ are both present and are linked together to form a —O—C₁₋₄alkylene-, —C₁₋₄alkylene-O—, —O—C₁₋₃alkylene-O—, —S—C₁₋₄alkylene-, —C₁₋₄alkylene-S— or —S—C₁₋₃alkylene-S— group, or
wherein at least one of R⁶ and R⁷ is present and represents halo.

There is also provided a compound of formula I or Ia (e.g. a compound of formula Ia) as defined herein (i.e. above or hereinafter) for use as a pharmaceutical. In such an embodiment of the invention, it is preferred that the compound does not represent certain compounds. That is, there is further provided a compound of formula I or Ia (for example, a compound of formula Ia) (or a salt thereof) as defined herein, but in which:

(i) when L represents —C(O)—:
R², R³ and R⁴ each represent hydrogen;
Y² represents 4-fluorophenyl;
X¹, X², X³ and X⁴ respectively represent CR⁵, CR⁶, CR⁷ and CR⁸,
R⁵, R⁷ and R⁸ each represent hydrogen;
R⁶ represents —OCH₃,
then Y¹ does not represent phenyl substituted at the 4-position with chloro, fluoro or methyl;
(ii) when L represents —S(O)₂—:
R², R³ and R⁴ each represent hydrogen;
Y² represents phenyl;
X¹, X², X³ and X⁴ respectively represent CR⁵, CR⁶, CR⁷ and CR⁸,
R⁵, R⁷ and R⁸ each represent hydrogen;
R⁶ represents fluoro,
then Y¹ does not represent phenyl substituted at the 4-position with methyl;
(iii) when L represents —S(O)₂—;
R², R³ and R⁴ each represent hydrogen;
Y² represents 4-methylphenyl;
X¹, X², X³ and X⁴ respectively represent CR⁵, CR⁶, CR⁷ and CR⁸,
R⁵, R⁷ and R⁸ each represent hydrogen;
R⁶ represents fluoro,
then Y¹ does not represent (unsubstituted) phenyl, or phenyl substituted at the 4-position with methyl or fluoro.

Further compounds for use as pharmaceuticals include those in which R⁶ and R⁷ are linked together with a —O—C₁₋₄alkylene-, —C₁₋₄alkylene-O— or —O—C₁₋₃alkylene-O— group as defined herein (or linked together with a —S—C₁₋₄alkylene-, —C₁₋₄alkylene-S— or —S—C₁₋₃alkylene-S-group as defined herein; but preferably the linking groups are the aforementioned oxygen-containing ones). Such an embodiment is also preferred for pharmaceutical compositions and/or formulations mentioned hereinafter. Such an embodiment may also be mentioned in respect of compounds of the invention for the medical uses described herein.

Yet further compounds for use as pharmaceuticals include those in which any one or more (e.g one) of R⁵ to R⁸ represent —OCH₃ (in particular, wherein R⁶ represents —OCH₃). Such an embodiment is also preferred for pharmaceutical compositions and/or formulations mentioned hereinafter and for the medical uses described herein.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin. For the avoidance of doubt, solvates are also included within the scope of the invention.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). When there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

Unless otherwise stated, the term C_{1-q} alkylene (where q is the upper limit of the range) defined herein may (in a similar manner to the definition of C_{1-q} alkyl) be straight-chain or, when there is a sufficient number of carbon atoms, be saturated or unsaturated (so forming, for example, an alkenylene or alkynylene linker group) or branched (although, alkylene groups are preferably straight-chained and saturated).

The term “halo”, when used herein, includes fluoro, chloro, bromo and iodo (for example, fluoro and chloro).

The term “aryl”, when used herein, includes C₆₋₁₄ (e.g. C₆₋₁₀) aromatic groups. Such groups may be monocyclic, bicyclic or tricyclic and, when polycyclic, be either wholly or partly aromatic. C₆₋₁₀ aryl groups that may be mentioned include phenyl, naphthyl, 1,2,3,4-tetrahydronaphthyl, indanyl, and the like (e.g. phenyl, naphthyl and the like). For the avoidance of doubt, the point of attachment of substituents on aryl groups may be via any carbon atom of the ring system (but is preferably via an aromatic ring of the aryl group).

The term “heteroaryl” (or heteroaromatic), when used herein, includes 5- to 14-membered heteroaromatic groups containing one or more heteroatoms selected from oxygen, nitrogen and/or sulfur. Such heteroaryl group may comprise one, two or three rings, of which at least one is aromatic. Substituents on heteroaryl/heteroaromatic groups may, where appropriate, be located on any atom in the ring system including a heteroatom. The point of attachment of heteroaryl/heteroaromatic groups may be via any atom in the ring system including (where appropriate) a heteroatom (preferably the point of linkage is via an atom of an aromatic ring of the heteroaryl group). Polycyclic heteroaryl/heteroaromatic groups may comprise a benzene ring fused to one or more further aromatic or non-aromatic heterocyclic rings, in which instances, the point of attachment of the polycyclic heteroaryl/heteroaromatic group may be via any ring including the benzene ring or the heteroaryl/heteroaromatic or heterocycloalkyl ring. Examples of heteroaryl/heteroaromatic groups that may be mentioned include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, quinolinyl, benzoimidazolyl and benzthiazolyl. The oxides of heteroaryl/heteroaromatic groups are also embraced within the scope of the invention (e.g. the N-oxide). As stated above, heteroaryl includes polycyclic (e.g. bicyclic) groups in which one ring is aromatic (and the other may or may not be aromatic). Hence, other heteroaryl groups that may be mentioned include e.g. benzo[1,3]dioxolyl, benzo[1,4]dioxinyl, 2,3-dihydrofuryl, 1,2,3,4-tetrahydro-quinolinyl and the like.

Heteroatoms that may be mentioned include phosphorus, silicon, boron and, preferably, oxygen, nitrogen and sulfur.

For the avoidance of doubt, when a term such as “R⁵ to R⁸” is employed herein, this will be understood by the skilled person to mean R⁵, R⁶, R⁷ and R⁸ inclusively.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. in which one or more hydrogen atoms are replaced by the hydrogen isotope deuterium.

All individual features (e.g. preferred features) mentioned herein may be taken in isolation or in combination with any other feature (including preferred features) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

In a particular embodiment, particular compounds of the invention (i.e. compounds of formula I or Ia, such as formula Ia) that may be mentioned (in respect of medical uses, use as pharmaceuticals, and pharmaceutical compositions and/or formulations mentioned herein) include those in which X¹, X², X³ and X⁴ each represents CR⁵, CR⁶, CR⁷, CR⁸ respectively or any one of X¹ to X³ alternatively and independently represents N. In particular, this embodiment may be combined with any other preference and/or embodiment relating to compounds of the invention (i.e. compounds of formula I or Ia, such as formula Ia) mentioned herein (such as the preferred embodiment mentioned directly below).

Preferred compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

R⁵, R⁶, R⁷ and R⁸ each independently represents hydrogen, halo, —R^{a}, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, —S(O)₂N(R^{r})R^{s}, or —SR^{t}; or any two R⁵, R⁶, R⁷ or R⁸ groups, when adjacent to one another, may be linked by a —C₁₋₅alkylene-, —O—C₁₋₄alkylene-, —C₁₋₄alkylene-O— or —O—C₁₋₃alkylene-O— group, which alkylene moieties are optionally substituted by one or more substituent selected from —F, C₁₋₃alkyl (optionally substituted by one or more —F) and ═O; but wherein at least one of R⁶ and R⁷ is present and represents —ORⁿ or —SR^{t}, or R⁶ and R⁷ are both present and are linked together to form a —O—C₁₋₄alkylene-, —C₁₋₄alkylene-O— or —O—C₁₋₃alkylene-O— group;

E¹ and E² each independently represent halo, —R^{a}, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, or —S(O)₂N(R^{r})R^{s}; and/or

any two E¹ or E² substituents, when adjacent to one another, may be linked to form a —C₁₋₅alkylene-, —O—C₁₋₄alkylene-, —C₁₋₄alkylene-O— or —O—C₁₋₃alkylene-O— group, which alkylene moieties are optionally substituted by one or more substituents selected from —F, C₁₋₃alkyl (optionally substituted by one or more —F) and ═O.

Other compounds of the invention (i.e. compounds of formula I or Ia, such as formula Ia) that may be mentioned (in respect of medical uses, use as pharmaceuticals, and pharmaceutical compositions and/or formulations mentioned herein, e.g. for the use(s) indicated herein) include those in which the compound of formula I is as hereinbefore defined, but in which:

either:
(a) R⁶ and R⁷ independently represent hydrogen, halo, C₂₋₆ alkyl (optionally substituted by one or more fluoro atoms), —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, —S(O)₂N(R^{r})R^{s}, or —SR^{t} (and e.g. at least one of R⁶ and R⁷ is present);
(b) R⁶ and R⁷ independently represent halo, C₁₋₆ alkyl (optionally substituted by one or more fluoro atoms), —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, —S(O)₂N(R^{r})R^{s}, or —SR^{t} (and e.g. at least one, and preferably both, of R⁶ and R⁷ is/are present); and/or, more preferably
(c) at least one of R⁶ and R⁷ is present and represents —ORⁿ or —SR^{t} (particularly —ORⁿ), or R⁶ and R⁷ are both present and are linked together to form a —O—C₁₋₄alkylene-, —C₁₋₄alkylene-O—, —O—C₁₋₃alkylene-O—, —S—C₁₋₄alkylene-, —C₁₋₄alkylene-S— or —S—C₁₋₃alkylene-S— group (more preferably to form a —O—C₁₋₄alkylene-, —C₁₋₄alkylene-O— or —O—C₁₋₃alkylene-O— group).

In the case of (a) above, it is preferred that R⁶ and R⁷ independently represent hydrogen, halo or C₂₋₆ alkyl (optionally substituted by one or more fluoro atoms) and at least one of R⁶ and R⁷ is present. In the case of (b) above, it is preferred that R⁶ and R⁷ independently represent halo or C₁₋₆ alkyl (optionally substituted by one or more fluoro atoms) and at least one, and preferably both, of R⁶ and R⁷ is/are present. In an embodiment of the invention the alternatives (a) and (b) (and the associated preferred embodiments of these alternatives) may be present (and not alternative (c)).

Preferred compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

X¹, X², X³ and X⁴ each represents CR⁵, CR⁶, CR⁷, CR⁸ respectively, or any one of X¹ to X⁴, alternatively and independently represents N
(in particular, in which X¹, X², X³ and X⁴ each represents CR⁵, CR⁶, CR⁷, CR⁸ respectively or any one of X¹ to X³ (such as X¹) alternatively and independently represents N);
E¹ and E² each independently represent halo, —R^{a}, —CN, —C(O)N(R^{c})R^{d}, —N(R^{f})R^{g}, —N(R^{h})C(O)Rⁱ, —N(R^{j})C(O)OR^{k}, —NO₂, —N(R^{l})S(O)₂—R^{m}, —OR^{o}, —S(O)ₘR^{q} or —S(O)₂N(R^{r})R^{s}; or
any two E¹ or E² substituents, when adjacent to one another, may be linked to form a —C₁₋₅alkylene-, —O—C₁₋₄alkylene-, —C₁₋₄alkylene-O— or a —O—C₁₋₃alkylene-O— group, which alkylene moieties are optionally substituted by one or more substituent selected from —F, C₁₋₃alkyl (optionally substituted by one or more —F) and ═O;

R¹ represents hydrogen, -Me, -Et, —CH₂F, —CHF₂ or —CF₃;

R² represents hydrogen, -Me or -Et;
R³ and R⁴ each independently represents hydrogen or -Me; or
R³ and R⁴ may be linked to form a further 3- to 4-membered ring;
R⁵, R⁶, R⁷ and R⁸ each independently represents hydrogen, halo, —R^{a}, —CN, —C(O)N(R^{c})R^{d}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —S(O)ₘR^{q}, —S(O)₂N(R^{r})R^{s} or —SR^{t}; or
any two R⁵, R⁶, R⁷ or R⁸ groups, when adjacent to one another, may be linked by a —C₁₋₅alkylene-, —O—C₁₋₄alkylene-, —C₁₋₄alkylene-O— or a —O—C₁₋₃alkylene-O— group, which alkylene moieties are optionally substituted by one or more —F or -Me;
each R^{a}, R^{b}, R^{c}, R^{d}, R^{e}, R^{f}, R^{g}, R^{h}, Rⁱ, R^{j}, R^{l}, Rⁿ, R^{o}, R^{q}, R^{r}, R^{s} and R^{t} independently represent hydrogen or C₁₋₃ alkyl (optionally substituted by one, two or three —F); or
any two R^{c} and R^{d}, R^{f} and R^{g} and/or R^{r} and R^{s} may be linked together with the nitrogen atom to which they are necessarily attached, to form a 4- to 6-membered monocyclic ring, which ring may optionally contain one additional heteroatom, and which ring may optionally be substituted by C₁₋₃alkyl (optionally substituted by one, two or three —F).

Particular compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

X², X³ and X⁴ each represents CR⁵, CR⁶, CR⁷, CR⁸ respectively; or
X¹, X², X³ and X⁴ each represents CR⁵, CR⁶, CR⁷, CR⁸ respectively and any one of X¹ to X³ (such as X¹) alternatively and independently represents N.

Preferred compounds of the invention (i.e. compounds of formula I or Ia, such as formula Ia) that may mentioned (in respect of medical uses, use as pharmaceuticals, and pharmaceutical compositions and/or formulations mentioned herein) include those in which:

at least one (i.e. one or both) of R⁶ and R⁷ is present and represents —ORⁿ or —SR^{t} (particularly, —ORⁿ); or
R⁶ and R⁷ are linked together to form a —O—C₁₋₄alkylene-, —C₁₋₄alkylene-O≠, —O—C₁₋₃alkylene-O—, —S—C₁₋₄alkylene-, —C₁₋₄alkylene-S— or —S—C₁₋₃alkylene-S— group; or, more preferably
at least one (i.e. one or both) of R⁶ and R⁷ is presents and represents —ORⁿ; or, preferably,
R⁶ and R⁷ are linked together to form a —O—C₁₋₄alkylene-, —C₁₋₄alkylene-O—, —O—C₁₋₃alkylene-O— (especially a —O—C₁₋₃alkylene-O— group).

Further preferred compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

when Y¹ and/or Y² represent aryl, they preferably represent optionally substituted phenyl or naphthyl;
when Y¹ and/or Y² represent heteroaryl, they preferably represent a 5- or 6-membered ring, in which the heteroaryl group contains one or two (e.g. one) heteroatom(s) preferably selected from nitrogen and oxygen;
Y¹ represents phenyl, furanyl (preferably, furan-2-yl), naphthyl or pyridyl (preferably, pyridin-3-yl);
Y² represents phenyl or pyridyl (preferably, pyridin-2-yl);
X¹, X², X³ and X⁴ each respectively represent CR⁵, CR⁶, CR⁷ and CR⁸;
each E¹ independently represents halo (e.g. —F, —Cl), —R^{a} or —ORⁿ; or
any two E¹ substituents, when adjacent to one another, may be linked to form a —O—C₁alkylene-O— group (which alkylene moiety is preferably unsubstituted);
each E² independently represents halo (e.g. —F, Cl), —N(R^{f})R^{g} or —ORⁿ; or
any two E² substituents, when adjacent to one another, may be linked to form a —O—C₁₋₂alkylene-O— group (which alkylene group is preferably unsubstituted);
R¹, R², R³, R⁴, R⁵ and R⁸ each independently represent hydrogen;
R⁶ and R⁷ each independently represent —ORⁿ; or
R⁶ and R⁷ are linked by a —O—C₁alkylene-O— group;
each R^{a}, R^{f}, R^{g} and Rⁿ independently represents hydrogen or, preferably, C₁₋₂ alkyl.

Still further preferred compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

E¹ and E² each independently represents halo, —R^{a}, —CN, —N(R^{f})R^{g}, —ORⁿ, or —S(O)ₘR^{q}; or
any two E¹ or E² substituents, when adjacent to one another, may be linked to form a —O—C₁₋₂alkylene-O— group, which alkylene moiety is optionally substituted by one or more groups selected from —F and -Me;
R¹ represents -Me, -or preferably, hydrogen;
R² represents -Me, -or preferably, hydrogen;
R³ and R⁴ each independently represents -Me, preferably one of R³ and R⁴ represents hydrogen and the other represents -Me, or, more preferably, both R³ and R⁴ represent hydrogen;
R⁵, R⁶, R⁷ and R⁸ each independently represents hydrogen, halo, —R^{a}, —CN, —N(R)R^{g}, —ORⁿ, —S(O)ₘR^{q}, or —SR^{t}; or
R⁶ and R⁷ are linked by a —O—C₁₋₂alkylene-O— group, which alkylene moiety is optionally substituted by one or more substituents selected from —F and -Me.

Yet further preferred compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

E¹ and E² each independently represents halo, —R^{a}, or —ORⁿ; or
any two E¹ or E² substituents, when adjacent to one another, may be linked to form a —O—C₁₋₂alkylene-O— group;
R¹ represents hydrogen;
R² represents -Me -or hydrogen;
R³ and R⁴ each independently represents -Me, preferably one of R³ and R⁴ represents hydrogen and the other represents -Me, or, more preferably, both R³ and R⁴ represent hydrogen;
R⁵, R⁶, R⁷ and R⁸ each independently represents hydrogen, halo, —R^{a}, —CN, —N(R^{f})R^{g}, —ORⁿ, —S(O)ₘR^{q}, or —SR^{t}; or
R⁶ and R⁷ are linked by a —O—C₁₋₂alkylene-O— group, which alkylene moiety is optionally substituted by one or more substituents selected from —F and -Me.

More preferred compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

Y¹ represents aryl or heteroaryl containing one heteroatom (e.g. one nitrogen or one oxygen atom), both of which are optionally substituted by one or more substituents selected from E¹;
Y² represents aryl or heteroaryl containing one heteroatom (e.g. one nitrogen atom), both of which are optionally substituted by one or more substituents selected from E²;
E¹ and E² each independently represents halo, —R^{a}, —CN, —ORⁿ or —S(O)ₘR^{q}; or
any two E¹ or E² substituents, when adjacent to one another, may be linked to form a —O—C₁₋₂alkylene-O— group, which alkylene moiety is optionally substituted by:

### (a) two —F;

### (b) one —F and one -Me;

### (c) two -Me; or

### (d) one -Me;

R⁵ and R⁸ both represent hydrogen.

More preferred compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

Y¹ represents aryl or heteroaryl containing one heteroatom (e.g. one nitrogen atom), both of which are optionally substituted by one or more substituents selected from E¹;
Y² represents aryl or heteroaryl containing one heteroatom (e.g. one nitrogen atom), both of which are optionally substituted by one or more substituents selected from E²;
X¹, X², X³ and X⁴ each represents CR⁵, CR⁶, CR⁷, CR⁸ respectively;
E¹ and E² each independently represents halo, —R^{a}, —CN, —ORⁿ or —S(O)ₘR^{q}; or
any two E¹ or E² substituents, when adjacent to one another, may be linked to form a —O—C₁₋₂alkylene-O— group, which alkylene moiety is optionally substituted by:

### (a) two —F;

### (b) one —F and one -Me;

### (c) two -Me; or

### (d) one -Me;

R⁵ and R⁸ both represent hydrogen.

Particular compounds of the invention (i.e. compounds of formula I or Ia, such as formula Ia) that may be mentioned (in respect of medical uses, use as pharmaceuticals, and pharmaceutical compositions and/or formulations mentioned herein) include those in which R⁵, R⁶, R⁷ and R⁸ are independently selected from halo, —R^{a}, —CN, —ORⁿ, —OC(O)R^{o}, —OS(O)₂R^{p}. In particular, compounds of the invention that may be mentioned include those in which: at least one (e.g. one or two, such as one) of R⁵, R⁶, R⁷ and R⁸ (such as R⁶) is —ORⁿ (and the remainder of R⁵, R⁶, R⁷ and R⁸ are hydrogen or methyl (e.g. hydrogen)); or in which R⁶ and R⁷ are linked to form —O—C₁₋₃alkylene-O— and R⁵ and R⁸ are hydrogen.

More particular compounds of the invention (i.e. compounds of formula I or Ia, such as formula Ia) that may be mentioned (in respect of medical uses, use as pharmaceuticals, and pharmaceutical compositions and/or formulations mentioned herein) include those in which:

L represents —C(O)—;
Y¹ represents phenyl or naphthyl, both of which are optionally substituted by one or more substituents selected from E¹;
R⁶ and R⁷ each independently represents —H, —F, —Cl, -Me, —CN, —ORⁿ or —SR^{t} provided that both do not represent H, i.e. at least one of R⁶ and R⁷ represents —ORⁿ or —SR^{t} (particularly, —ORⁿ), or R⁶ and R⁷ are linked by a —O—C₁₋₃alkylene-O— group (e.g. —O—C₁alkylene-O— group), which group (alkylene moiety) is optionally substituted by

### (a) two —F,

### (b) one —F and one -Me

### (c) two -Me, or

### (d) one -Me

(for example, said group (alkylene moiety) is not substituted); or
more preferably, R⁶ and R⁷ are linked together to form a —O—C₁alkylene-O group in which the alkylene moiety is optionally substituted by one or more substituents selected from —F, —CH₃ and —CF₃ (but is preferably unsubstituted).

Compounds of the invention (i.e. compounds of formula I or Ia, such as formula Ia) that may be mentioned (in respect of medical uses, use as pharmaceuticals, and pharmaceutical compositions and/or formulations mentioned herein) include those of formula II:

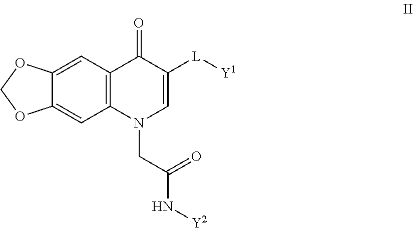

i.e. in which R⁶ and R⁷ are linked together to form a —OCH₂—O— moiety and wherein L, Y¹ and Y² are as herein before defined.

Further compounds of the invention that may be mentioned (in respect of medical uses, use as pharmaceuticals, and pharmaceutical compositions and/or formulations mentioned herein) include compounds of formula II in which:

Y¹ represents phenyl, furanyl (preferably, furan-2-yl), naphthyl or pyridyl (preferably, pyridin-3-yl);

Y² represents phenyl, or pyridyl (perferably, pyridin-2-yl);
each E¹ independently represents —F, —Cl, —R^{a}, or —ORⁿ; or
any two E¹ substituents, when adjacent to one another, may be linked to form a —O—C₁alkylene-O— group;

each E² independently represents —F, Cl, —N(R^{f})R^{g}, or —ORⁿ; or

any two E² substituents, when adjacent to one another, may be linked to form a —O—C₁₋₂alkylene-O— group;
each R^{a}, R^{f}, R^{g} and Rⁿ independently represents C₁₋₂ alkyl (optionally substituted by one or more —F).

Yet further compounds of the invention that may be mentioned (in respect of medical uses, use as pharmaceuticals, and pharmaceutical compositions and/or formulations mentioned herein) include those of formula IIa, wherein compounds of formula IIa take the same definition as compounds of formula I (as defined in any of the embodiments above) except that any one of X¹ to X³ (such as X¹) represents N, X⁴ represents CR⁸ and the remainder of X¹, X² and X³ each represents CR⁵, CR⁶, CR⁷ respectively.

Yet further compounds of the invention that may be mentioned (in respect of medical uses, use as pharmaceuticals, and pharmaceutical compositions and/or formulations mentioned herein) include those of formula IIb, wherein compounds of formula IIb take the same definition as compounds of formula Ia (as defined in any of the embodiments above) except that any one of X¹ to X³ (such as X¹) represents N, X⁴ represents CR⁸ and the remainder of X¹, X² and X³ each represents CR⁵, CR⁶, CR⁷ respectively.

More preferred compounds of formula IIa or IIb include those in which:

L represents —C(O)—;
Y¹ represents phenyl or naphthyl, both of which are optionally substituted by one or more (e.g. one) substituents selected from E¹ (such as fluoro);
R⁵, R⁶, R⁷ and R⁸ each independently represents hydrogen, halo (such as fluoro), —ORⁿ or —SR^{t} (particularly, H or —ORⁿ, such as wherein Rⁿ represents H or methyl) (for example, wherein one of R⁶ and R⁷ represents —ORⁿ and the remainder of R⁵, R⁶, R⁷ and R⁸ represents H);
R² represents hydrogen;
both R³ and R⁴ represent hydrogen;
Y² represents phenyl, optionally substituted with one or more (e.g. one) substituents selected from E²;
E² each independently represents halo (such as fluoro or, particularly, chloro) or
R^{a} (such as wherein R^{a} represents methyl, optionally substituted with one or more fluoro (e.g. —CF₃)).

Particularly preferred compounds of the invention (i.e. compounds of formula I, Ia, II, IIa and/or IIb) that may be mentioned (in respect of medical uses, use as pharmaceuticals, and pharmaceutical compositions and/or formulations mentioned herein) include those of the examples described hereinafter.

Compounds of the invention (including compounds of formula I, Ia, II, IIa and/or IIb) may be made in accordance with techniques that are well known to those skilled in the art, for example as described hereinafter.

According to a further aspect of the invention there is provided a process for the preparation of a compound of formula I or Ia (e.g. a compound of formula Ia), which process comprises:

(i) reaction of a compound of formula III

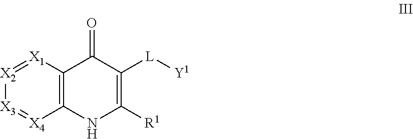

wherein X¹ to X⁴ (inclusive), R¹, L and Y¹ are as hereinbefore defined, with a compound of formula IV

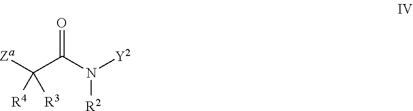

wherein R² to R⁴ (inclusive) and Y² are as hereinbefore defined and Z^{a} represents a suitable leaving group (e.g. —Cl, —Br, mesylate or the like), under conditions known to those skilled in the art, for example in the presence of a suitable base (such as a non-nucleophilic base, e.g. sodium hydride) and in the presence of a suitable solvent (such as an aprotic solvent, e.g. DMF);
(ii) reaction of a compound of formula III as hereinbefore defined with a compound of formula V

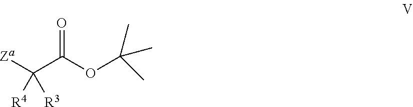

wherein R³, R⁴ and Z^{a} are as hereinbefore defined, in the presence of a suitable base (preferably a non-nucleophilic base, such a potassium carbonate) and in the presence of a suitable solvent (such as DMF), followed by generation of the free carboxylic acid under conditions known to those skilled in the art, for example acid in the presence of a suitable acid (such as strong acid, e.g. TFA) and in the presence of a suitable solvent (such as dichloromethane), and then (followed by) either:
(a) reaction with a compound of formula VI

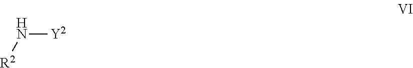

wherein R² and Y² are as hereinbefore defined, in the presence of a suitable peptide coupling reagent (such as a carbodiimide (e.g. DCC) or a phosphonium salt (e.g. HATU)) and in the presence of a suitable solvent (such as DMSO), or
(b) conversion to an acid chloride under conditions known to those skilled in the art, such in the presence of thionyl chloride and in the presence of a suitable solvent (such as an aprotic solvent, e.g. dichloromethane), and then followed by reaction with a compound of formula VI as herein before defined under conditions known to those skilled in the art, such as in the presence of a suitable solvent (such as an aprotic solvent, e.g. dichloromethane) and in the presence of a suitable base (such as a non-nucleophilic base, e.g. pyridine). Those skilled in the art will understand that esters other than tert-butyl may be used in compounds of formula V and that these may be deprotected (i.e. to generate the free carboxylic acid) using different conditions, e.g. hydrolysis for primary alkyl esters and hydrogenation of benzyl esters;
(iii) for compounds of formula I (for example, wherein L is represented by —C(O)—), reaction of a compound of formula VII

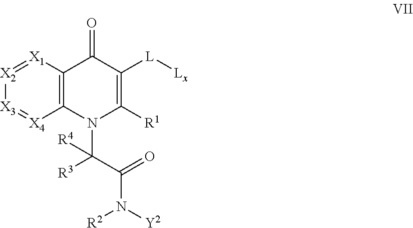

wherein X¹ to X⁴, L, R¹ to R⁴ and Y² are as hereinbefore defined and Lₓ represents a suitable leaving group (such as —N(OMe)Me, halo (e.g. —Cl) or —O—C₁₋₂alkyl), with a compound of formula VIII

Y¹-Mₓ VIII

wherein Y¹ is as hereinbefore defined and Mₓ represents a either a suitable metal (such as Li) or a suitable metal halide (such as —MgBr or —ZnCl), in the presence of a suitable solvent (such as an aprotic solvent, e.g. THF). The skilled person will recognise that, wherein R² is represented by H, R² may be replaced with suitable nitrogen protecting group (e.g. benzyl) which may then be removed, under conditions known to those skilled in the art, to obtain the required product;
(iv) for compounds of formula I (in particular, wherein L is represented by —C(O)—), reaction of a compound of formula IX

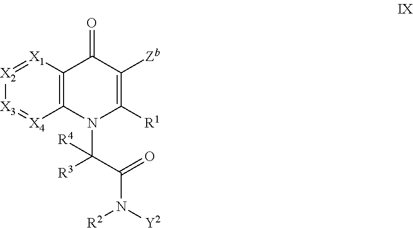

wherein X¹ to X⁴, R¹ to R⁴ and Y² are as hereinbefore defined and Z^{b} represents halo (e.g. —Br, —Cl or —I), either with:
(a) a suitable transmetallation agent, such as an alkyl metal (e.g. t-butyl lithium) or alkyl metal halide (e.g. iPrMgCl), or
(b) a metal capable of forming a suitable metal halide (such as magnesium, thus converting —Z^{b} to —MgZ^{b}),
and in the presence of a suitable solvent (such as a aprotic solvent, e.g. THF) and (followed by reaction with) a compound of formula X

Y¹-L-Lₓ X

wherein Y¹ and Lₓ are as hereinbefore defined, in the presence of a suitable solvent (such as an aprotic solvent, e.g. THF). The skilled person will recognise that, wherein R² is represented by H, R² may be replaced with suitable nitrogen protecting group (e.g. benzyl) which may then be removed, under conditions known to those skilled in the art, to obtain the required product;
(v) for compounds of formula I (in particular, wherein R¹ is H and/or at least one of X₂ and X₄ represents N), reaction of a compound of formula XI,

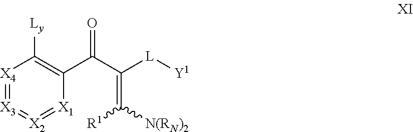

wherein X¹ to X⁴ and Y¹ are as hereinbefore defined, L_{y} represents a suitable leaving group (such as halo (e.g. —Cl)) and each R_{N} represents a suitable substituent (such as C₁₋₄ alkyl (e.g. methyl) or wherein both R_{N} groups are linked to form a C₃₋₆ heteroalkyl containing the N to which they are attached), with a compound of formula XII,

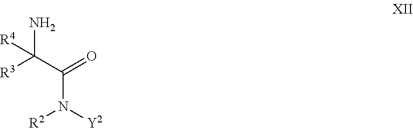

wherein R² to R⁴ and Y² are as hereinbefore defined, under conditions known to those skilled in the art, such as in the presence of a suitable solvent (such as MeOH) and a suitable base (such as NaOMe) and, optionally, at increased temperature.

Compounds of formula III (in particular, in which R¹ represents H) may be prepared by intramolecular reaction of:

a compound of formula XIII

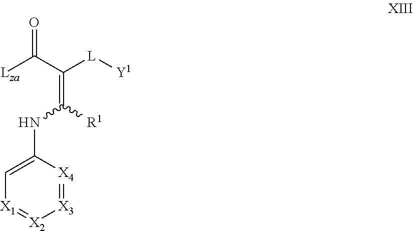

or; a compound of formula XIV

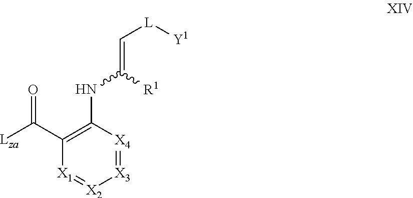

wherein X¹ to X⁴ and Y¹ are as hereinbefore defined and each L_{za} independently represents a suitable leaving group (such as —O—C₁₋₂alkyl), under conditions known to those skilled in the art.

Compounds of formula III may also be prepared by reaction of a compound of formula XV

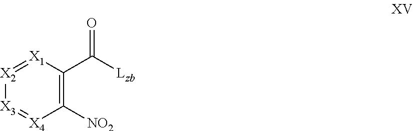

wherein X¹ to X⁴ are as hereinbefore defined and L_{Zb} represents a suitable leaving group (such as halo (e.g. —Cl) or —O—C₁₋₂alkyl), with a compound of formula XVI

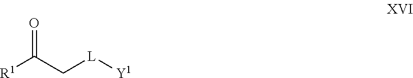

wherein R¹, L and Y¹ are as hereinbefore defined, in the presence of a suitable base (such as a non-nucleophilic base, e.g. LDA) and in the presence of a suitable solvent (such as THF) at reduced temperature (e.g. −78° C.), and (followed by) hydrogenation, under conditions known to those skilled in the art (for example, under an atmosphere of hydrogen gas and in the presence of a suitable catalyst, such as Pd—C, and a suitable solvent, such as ethyl acetate), and subsequent intramolecular cyclisation.

Compounds of formula III (in particular, wherein in which R¹ represents H and/or at least one of X₂ or X₄ represents N) may also be prepared by reaction of a compound of formula XI as hereinbefore defined with a suitably-protected amine (such as tert-octylamine) under conditions known to those skilled in the art, such as in the presence of a suitable solvent (such as MeOH) and a suitable base (such as NaOMe) and, optionally, at increased temperature, followed by removal of the amine protecting group under conditions known to those skilled in the art.

Compounds of formulae IV, V, VI, VII, VIII, IX, X, XI, XII, XIII, XIV, XV and XVI are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

The substituents R⁶ to R⁸ (i.e. substituents on X¹ to X⁴), R¹ to R⁴, Y¹, Y², L, Z^{a}, Z^{b}, Lₓ, L_{y}, L_{za} and L_{zb} as hereinbefore defined may be modified one or more times, after or during the processes described above for preparation of compounds of formula I and/or III by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. In the case where X¹ to X⁴ represents CR⁶, CR⁶, CR⁷ and CR⁶ respectively and R⁵ to R⁸ represents a Cl or F group, such groups may be inter-converted (or converted from another halo group) one or more times, after or during the processes described above for the preparation of compounds of formula I and/or formula III. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures using conventional techniques.

It will be appreciated by those skilled in the art that, in the processes described above and hereinafter, the functional groups of intermediate compounds may need to be protected by protecting groups. For example the nitrogen atom of an amide, quinolone or naphthyridone may need to be protected. Suitable nitrogen-protecting groups include those which form:

(i) carbamate groups (i.e. alkoxy- or aryloxy-carbonyl groups);
(ii) amide groups (e.g. acetyl groups);
(iii) N-alkyl groups (e.g. benzyl, tert-octyl or SEM groups);
(iv) N-sulfonyl groups (e.g. N-arylsulfonyl groups);
(v) N-phosphinyl and N-phosphoryl groups (e.g. diarylphosphinyl and diarylphosphoryl groups);
(vi) N-silyl group (e.g. a N-trimethylsilyl group); or
(vii) —C(O)O⁻ (which may be formed in situ by reaction with an organolithium base and CO₂).

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3rd edition, T. W. Greene & P.G.M. Wutz, Wiley-Interscience (1999).

### Medical and Pharmaceutical Uses

As discussed hereinbefore, compounds of the invention are indicated as pharmaceuticals. According to a further aspect of the invention there may be provided a compound of the invention, as hereinbefore defined, for use as a pharmaceutical (e.g. with certain provisos).

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time, following enteral or parenteral administration (e.g. oral or parenteral administration). All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds, which possess pharmacological activity.

It is stated herein that the compounds of the invention may be useful in the treatment, prevention and/or prophylaxis of autoimmune diseases and/or inflammatory (e.g. chronic inflammatory) diseases. For the purposes of this specification, and for the avoidance of doubt, the term “treatment” includes treatment per se, prevention and prophylaxis.

In an alternative embodiment, compounds of the invention may be useful in the treatment of autoimmune diseases and/or of chronic inflammatory disorders and/or the therapeutic treatment of inflammation (or inflammatory symptoms) associated with such disorders.

Preferably, the autoimmune disease is selected from rheumatoid arthritis, multiple sclerosis, inflammatory bowel disease including Crohn's disease and ulcerative colitis, systemic lupus erythematosus, autoimmune uveitis, type I diabetes, dermatomyesitis, Goodpasteure's syndrome, Graves' disease, Guillian-Barré Syndrome (GBS), Hashimotos Disease, Mixed connective tissue disease, Myasthenia gravis, Pemphigus vulgaris, Pernicious anemia, Psoriasis, Polymyositis, Primary biliary cirrhosis, Sjögren's syndrome, Giant cell arteritis, ulcerative colitis, vasculitis, Wegener's granulomatosis, Churg-Strauss syndrome and iopathic thrombocytopenic purpura. Most preferably the autoimmune disease is selected from rheumatoid arthritis and multiple sclerosis. Preferably, the inflammatory (e.g. chronic inflammatory) disease is selected from include celiac disease, vasculitis, lupus, chronic obstructive pulmonary disease (COPD), irritable bowel disease, atherosclerosis, arthritis and psoriasis.

Compounds of the invention will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form.

Compounds of the invention may be administered alone, but are preferably administered by way of known pharmaceutical compositions/formulations, including tablets, capsules or elixirs for oral administration, suppositories for rectal administration, sterile solutions or suspensions for parenteral or intramuscular administration, and the like.

Compounds of the invention (i.e. compounds that increase a NADPH oxidase complex activity) may be administered in the form of tablets or capsules, e.g., time-release capsules that are taken orally. Alternatively, the compounds of the invention may be in a liquid form and may be taken orally or by injection. The compounds of the invention may also be in the form of suppositories, or, creams, gels, and foams e.g. that can be applied to the skin. In addition, they may be in the form of an inhalant that is applied nasally.

Such compositions/formulations may be prepared in accordance with standard and/or accepted pharmaceutical practice.

According to a further aspect of the invention there is thus provided a pharmaceutical composition/formulation including a compound of the invention, as hereinbefore defined, optionally in admixture with a pharmaceutically acceptable adjuvant, diluent and/or carrier. Such compositions/formulations may be of use in the treatment, prevention and/or prophylaxis of autoimmune diseases and/or inflammatory (e.g. chronic inflammatory) diseases (e.g. such as those defined herein).

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The invention further provides a process for the preparation of a pharmaceutical formulation, as hereinbefore defined, which process comprises bringing into association a compound of the invention, as hereinbefore defined, or a pharmaceutically acceptable salt thereof with a pharmaceutically-acceptable adjuvant, diluent or carrier.

In yet another aspect the present invention provides methods for the treatment, prevention and/or prophylaxis of autoimmune diseases and/or inflammatory (e.g. chronic inflammatory) diseases (such as those described herein) comprising administering a therapeutically effective amount of a compound of the invention to a subject (e.g. patient) in need of such treatment.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention may also be combined with other therapeutic agents that are useful in the treatment, prevention and/or prophylaxis of autoimmune diseases and/or inflammatory (e.g. chronic inflammatory) diseases.

According to a further aspect of the invention, there is provided a combination product comprising:

(A) a compound of the invention, as hereinbefore defined; and
(B) another therapeutic agent that is useful in the in the treatment, prevention and/or prophylaxis of autoimmune diseases and/or inflammatory (e.g. chronic inflammatory) diseases,
wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the treatment, prevention and/or prophylaxis of autoimmune diseases and/or inflammatory diseases, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the treatment, prevention and/or prophylaxis of autoimmune diseases and/or inflammatory (e.g. chronic inflammatory) diseases in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined, or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages (and subcutanoues dosages, although these dosages may be relatively lower) may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

### Treating an Autoimmune Condition

As stated herein, the invention provides compounds, compositions and methods for treating/preventing autoimmune conditions, like arthritis, etc. The therapeutic effect of the compounds of the invention may be mediated by an increase in the capacity of a NADPH oxidase complex to produce ROS. Hence, where it is stated herein that the compounds of the invention may be useful in the treatment/prevention of “autoimmune diseases and/or inflammatory (e.g. chronic inflammatory) diseases”, this may also be taken to mean in the treatment/prevention of a disease or condition wherein increased NADPH oxidase activity (and therefore increased production of reactive oxygen species (ROS)) is desired and/or required. That is, the diseases/conditions mentioned herein may be treated/prevented from/by production of ROS by a NADPH oxidase complex (and compounds of the invention may increase NADPH oxidase activity).

As stated herein, compounds of the invention may be administered at any suitable dose, for instance, which is sufficient to increase NADPH oxidase activity in cells. Such doses can be taken over a period of years to prevent and/or delay the progression of autoimmune diseases or symptoms of inflammation (e.g. chronic inflammation). Doses can be selected based on the effectiveness and toxicity of the particular agent using standard pharmacology techniques.

Identifying Agents that Modify NADPH Oxidase Activity

The invention provides methods and materials for identifying agents that modulate reactivity of autoreactive cells by enhancing ROS production from a NADPH oxidase complex. A NADPH oxidase complex activity can be determined after incubation of a test agent and NADPH oxidase sufficient cells and compared to ROS production of the same cells in absence of test agent. Such cells can be from human or non-human mammals.

Agents inducing ROS production can also cause activation of Reactive Nitrogen Species (NOX) by stimulation of iNOS, eNOS or nNOS (Zhou and Zhu 2009). Hence, novel compounds, compositions and methods described herein could also be directed for activation of these enzymes for production of NOX with the same therapeutic purpose as that of NADPH oxidase produced ROS.

Described herein are novel ROS inducing compounds/compositions for activation of a NADPH oxidase complex to produce ROS. These compounds/compositions are shown to be effective for treatment and/or prophylaxis of autoimmune diseases.

Another aspect of the invention provides a method for identifying an agent being potentially suitable for the prophylaxis, prevention and/or treatment of an autoimmune disease and/or an inflammatory (e.g. chronic inflammatory) disease.

The method comprises the steps: (a) providing a biochemical or cellular assay able to determine NADPH oxidase activity; (b) Determination of the level of NADPH oxidase activity in the presence of a test agent; (c) Evaluation of whether or not the level is greater than a control level of NADPH oxidase activity, where the control level is the NADPH oxidase activity in the absence of test agent; (d) Identification of the agent as being potentially suitable for the prophylaxis, prevention and/or treatment of autoimmune diseases and/or inflammatory (e.g. chronic inflammatory) disease when the level is greater than the control level. The level of NADPH oxidase activity can be determined by measuring the level of cellular ROS production measured by luminescence from luminol/Isoluminol, fluorescence of dihydrorhodamine-123, absorbance of cytochrome C or other ROS detection methods in vitro and in vivo; (e) Identified ROS producing agents are evaluated for effect on immunological autoreactivity such as modulation of T cell activation measured by quantification of cell surface markers including, but not limited to, T cell markers e.g. T cell receptor CD3, CD4, markers for activated T cells (e.g. CD25, CD69 and MHC class II), presense of regulatory T cells (FoxP3), T cell proliferation and cytokine production (including but not limited to TNFa, IFN-g, IL1, IL6, IL17 and IL-2). Substances that, by inducing ROS production, significantly alter T cell activation compared to control cells are regarded as suitable agents for down regulation of T cell autoreactivity.

It is contemplated that any method or composition described herein can be implemented with respect to any other method or composition described herein.

Unless otherwise defined, all technical and scientific terms used herein have the same meaning as commonly understood by one of ordinary skill in the art to which this invention pertains.

The invention is illustrated by way of the following examples. As the examples are merely illustrative, they are not intended to limit the scope of the invention, but may be considered to embody certain aspects of the invention. Certain aspects of the invention (and specific examples) will now be described, with reference to the following figures (in which Substance A corresponds to Compound 84; and Substance B corresponds to Compound I, both described hereinafter):

### FIGURE LEGENDS

FIG. 1: Adoptive transfer of arthritogenic cells incubated with H₂O₂ at different concentrations in rats. Arthritogenic cells were treated with H₂O₂ (176-1.76 μM) for 20 min and A) granulocytes and B) T cells (see FIGS. 1A and 1B) were investigated for intracellular ROS using a DHR-123 based flow cytometric assay. Intracellular ROS reflected amount of H₂O₂ added (n=3). Arthritogenic cells were cultured in presence of Con A and w/wo H₂O₂ (176-1.76 μM) for 44 hours and then transferred to naïve recipient rats (n=7-9). A significant reduction in C) mean arthritis severity in recipient rats can be seen (see FIG. 1C) when arthritogenic T cells are cultured in presence of 176 μM and 88 μM but at lower concentrations of H₂O₂ the effect is lost. D) A significant effect of 176 μM on arthritogenic T cells can be seen (FIG. 1D).

All data is presented as mean±SEM. * represent a p-value <0.05, ** represent a p-value <0.01, *** represent a p-value <0.001.

FIG. 2: ROS production and effect on T cell arthritogeniciy of ROS inducing substances. A-B). Thioglycolate recruited rat granulocytes (in accordance with the procedure in Biological Example 3) were treated with 25 μM substance and measurement of ROS production was initiated immediately using an isoluminol based chemiluminescence assay (FIGS. 2A and 2B). ROS production was clearly enhanced when treated with substance compared to DMSO control. C-D) Arthritogenic rat splenocytes were treated with 12.5 μM substance during 44 hours ex vivo incubation in presence of ConA (FIGS. 2C and 2D). Following incubation, cells were washed and injected i.p. into naive recipient rats (n=8-11). Disease severity in recipient rats was followed by investigation of macroscopic swelling and redness of the rat joints. Treatment with substance during ex vivo stimulation prevented transfer of arthritogenic T cells.

All data is presented as mean±SEM. * represent a p-value <0.05, ** represent a p-value <0.01, *** represent a p-value <0.001.

### EXAMPLES

The invention is illustrated by way of the following examples, in which the following abbreviations may be employed:

aq aqueous
DMF dimethylformamide
DMSO dimethyl sulfoxide
EtOAc ethyl acetate
EtOH ethanol
MeOH methanol
MeCN acetonitrile
Pd—C palladium on carbon
rt room temperature
sat saturated
TFA trifluoroacetic acid
THF tetrahydrofuran

Starting materials and chemical reagents specified in the syntheses described below are commercially available, e.g. from Sigma-Aldrich Fine Chemicals.

In the event that there is a discrepancy between nomenclature and any compounds depicted graphically, then it is the latter that presides (unless contradicted by any experimental details that may be given or unless it is clear from the context).

### Example 1

### N-(4-Chlorophenyl)-2-[6,7-methylenedioxy-3-(2-naphthoyl)-4-oxo-1-quinolinyl]-acetamide

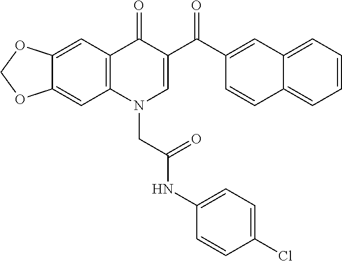 [SMILES: O=C(Cn1cc(C(=O)c2ccc3ccccc3c2)c(=O)c2cc3c(cc21)OCO3)Nc1ccc(Cl)cc1]

### (a) 2-Naphthoylacetic acid ethyl ester

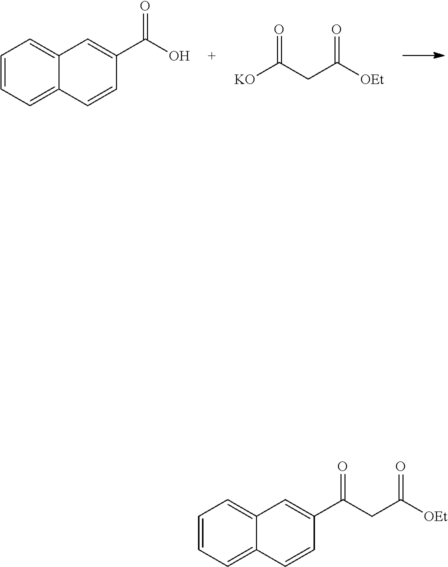

(i) Carbonyldiimidazole (16.9 g, 104.54 mmol) was added in portions to a mixture of 2-naphthoic acid (15.0 g, 87.12 mmol) and anhydrous THF (120 ml). The mixture was stirred at rt overnight.
(ii) MgCl₂ (20.7 g, 217 mmol) was added in portions to a mixture of potassium ethyl malonate (29.65 g, 174 mmol), Et₃N (36.6 ml, 261 mmol) and anhydrous MeCN (420 ml) keeping the temperature below 20° C. The mixture was stirred at rt for 4 h and cooled in an ice-bath. The solution from step (i) was added dropwise and the mixture was stirred at rt overnight and concentrated. Toluene (250 ml) was added. The mixture was cooled in an ice-bath and 4 M HCl (140 mL) was carefully added. The mixture was allowed to warm to rt and the layers were separated. The aqueous layer was extracted with EtOAc and the combined organic layers were washed with water, brine, and dried (Na₂SO₄). Concentration and purification by chromatography gave the sub-title compound (20.05 g, 95%).

### (b) 3-Ethoxy-2-(2-naphthoyl)acrylic acid ethyl ester

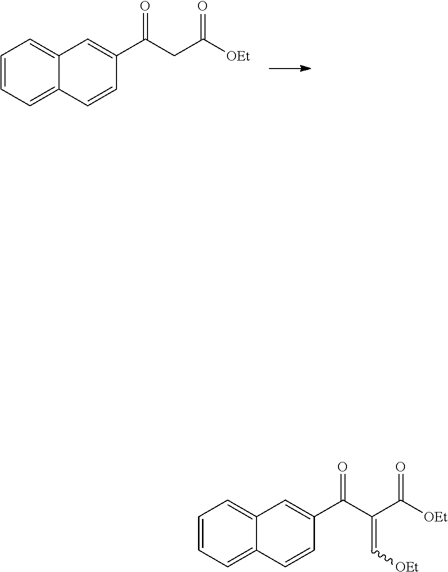 [SMILES: CCOC(=O)CC(=O)c1ccc2ccccc2c1 ; CCOC=C(C(=O)OCC)C(=O)c1ccc2ccccc2c1]

A mixture of 2-naphthoylacetic acid ethyl ester (20 g, 82.8 mmol), CH(OEt)₃ (20.6 mL, 124 mmol) and acetic anhydride (15.6 mL, 165.5 mmol) was heated at reflux for 22 h and concentrated. The material was used in the following step without further purification.

### (c) 3-(3,4-Methylenedioxyphenylamino)-2-(2-naphthol)acrylic acid ethyl ester

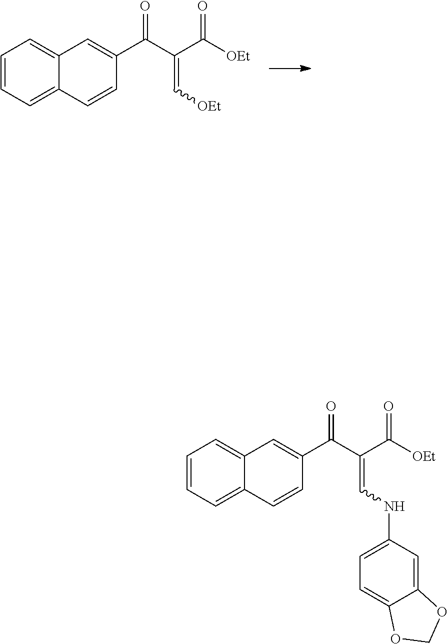 [SMILES: CCOC=C(C(=O)OCC)C(=O)c1ccc2ccccc2c1 ; CCOC(=O)C(=CNc1ccc2c(c1)OCO2)C(=O)c1ccc2ccccc2c1]

3,4-Methylenedioxyaniline (5.0 g, 36.6 mmol) was added to a mixture of 3-ethoxy-2-(2-naphthoyl)acrylic acid ethyl ester (10.9 g, 36.6 mmol) and toluene (70 mL). The mixture was heated at reflux for 3.75 h, cooled and concentrated. Purification of the residue by chromatography gave the sub-title compound (10.2 g, 72%).

### (d) 6,7-Methylenedioxy-3-(2-naphthoyl)-4-oxoquinoline

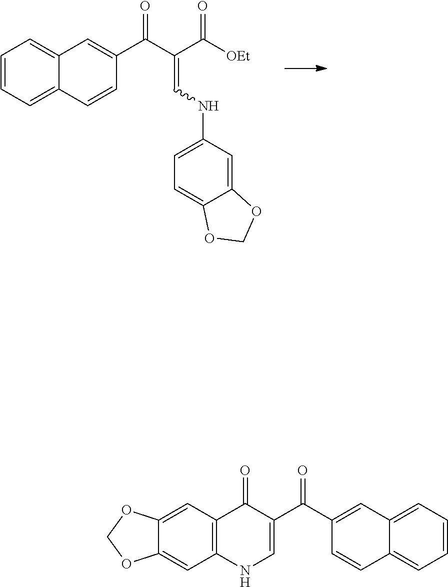 [SMILES: CCOC(=O)C(=CNc1ccc2c(c1)OCO2)C(=O)c1ccc2ccccc2c1 ; O=C(c1ccc2ccccc2c1)c1c[nH]c2cc3c(cc2c1=O)OCO3]

3-(3,4-Methylenedioxyphenylamino)-2-(2-naphthoyl)acrylic acid ethyl ester (5.6 g, 14.4 mmol) was added to diphenylether (35 mL) at 240° C. The mixture was heated at reflux for 6 h and allowed to cool to rt. The precipitate was collected and washed with petroleum ether 40-60.

### (e) 2-Chloro-N-(4-chlorophenyl)acetamide

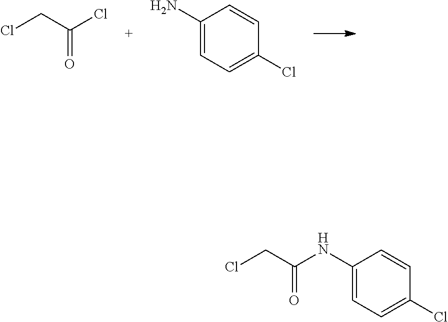 [SMILES: O=C(Cl)CCl ; Nc1ccc(Cl)cc1 ; O=C(CCl)Nc1ccc(Cl)cc1]

A mixture of 4-chloraniline (2.6 g, 20 mmol), NaHCO₃ (2.0 g, 24 mmol) and acetone (30 mL) was added over 30 min to a mixture of 2-chloroacetylchloride 2.5 g (22 mmol) in acetone (10 mL). The mixture was stirred for 1 d and the solid formed was collected, washed with water and dried to give the sub-title compound (3.1 g, 76%).

### (f) N-(4-Chlorophenyl)-2-[6,7-methylenedioxy-3-(2-naphthoyl)-4-oxo-1-quinolinyl]acetamide

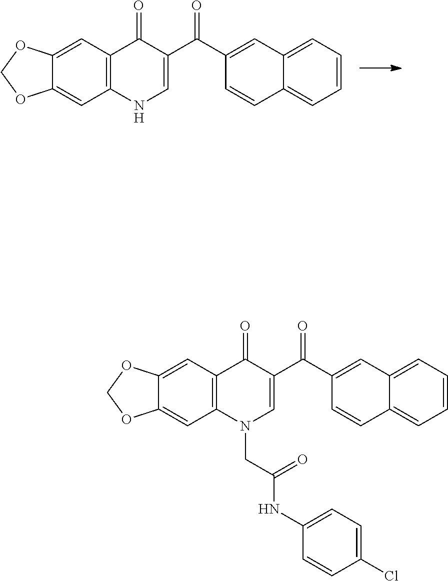 [SMILES: O=C(c1ccc2ccccc2c1)c1c[nH]c2cc3c(cc2c1=O)OCO3 ; O=C(Cn1cc(C(=O)c2ccc3ccccc3c2)c(=O)c2cc3c(cc21)OCO3)Nc1ccc(Cl)cc1]

NaH (60% in mineral oil, 14 mg, 0.35 mmol) was added to a mixture of 6,7-methylenedioxy-3-(2-naphthoyl)-4-oxoquinoline and DMF (6 mL) at rt. After 15 min 2-chloro-N-(4-chlorophenyl)acetamide (71 mg, 0.35 mmol) was added. The mixture was stirred at 30 min at rt and 2 h at 50° C. The mixture was concentrated and the residue purified by chromatography to give the sub-title compound (82 mg, 55%).

¹H NMR (500 MHz, DMSO-d₆) δ: 11.04 (bs, 1H), 8.47 (s, 1H), 8.33 (s, 1H), 8.11-7.94 (m, 3H), 7.82 (s, 1H), 7.74-7.47 (m, 5H), 7.46-7.27 (m, 3H), 6.20 (s, 2H), 5.33 (s, 2H).

### Examples 2-13

The title compounds where prepared in accordance with Example 1, using the appropriately substituted arylamine in step (e), see Table 1.

TABLE 1

| | Chemical structure |
|---|---|
| | Name |
| Example | ¹H NMR (400 MHz, DMSO-d₆) δ |
| 2 | 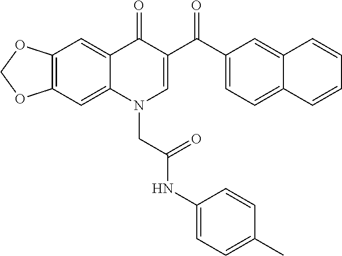 [SMILES: Cc1ccc(NC(=O)Cn2cc(C(=O)c3ccc4ccccc4c3)c(=O)c3cc4c(cc32)OCO4)cc1] |
| | 2-[6,7-Methylenedioxy-3-(2-naphthoyl)-4-oxo-1-quinolinyl]-N-p- |
| | tolylacetamide |
| | 10.37 (1H, s) 8.43 (1H, s) 8.29 (1H, s) 8.02 (1H, d, J = 8.1 Hz) |
| | 7.97-7.93 (2H, m) 7.78 (1H, dd, J = 8.6, 1.6 Hz) 7.63-7.59 (1H, m) |
| | 7.56-7.52 (1H, m) 7.50 (1H, s) 7.47 (1H, s) |
| 3 | 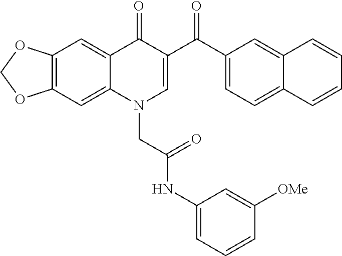 [SMILES: COc1cccc(NC(=O)Cn2cc(C(=O)c3ccc4ccccc4c3)c(=O)c3cc4c(cc32)OCO4)c1] |
| | N-m-Anisyl-2-[6,7-methylenedioxy-3-(2-naphthoyl)-4-oxo-1- |
| | quinolinyl]acetamide |
| | 10.47 (1H, s) 8.43 (1H, s) 8.29 (1H, s) 8.02 (1H, d, J = 8.0 Hz) |
| | 7.97-7.94 (2H, m) 7.78 (1H, dd, J = 8.6, 1.8 Hz) 7.63-7.59 (1H, m) |
| | 7.56-7.52 (1H, m) 7.50 (1H, s) 7.30-7.29 (1H, m) 7.23-7.19 (2H, |
| | m) 7.11-7.08 (1H, m) 6.64 (1H, dd, J = 8.4, 2.4 Hz) 6.17 (2H, s) |
| | 5.24 (2H, s) 3.69 (3H, s) |
| 4 | 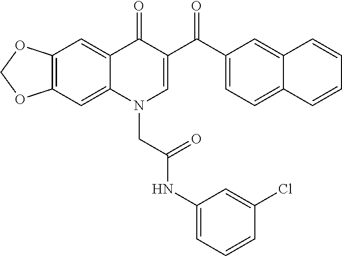 [SMILES: O=C(Cn1cc(C(=O)c2ccc3ccccc3c2)c(=O)c2cc3c(cc21)OCO3)Nc1cccc(Cl)c1] |
| | N-(3-chlorophenyl)-2-[6,7-methylenedioxy-3-(2-naphthoyl)-4- |
| | oxo-1-quinolinyl]acetamide |
| | 10.68 (1H, s) 8.47 (1H, s) 8.32 (1H, s) 8.06 (1H, d, J = 8.2 Hz) |
| | 7.97-7.94 (2H, m) 7.84-7.79 (2H, m) 7.68-7.62 (1H, m) 7.61-7.55 |
| | (1H, m) 7.54 (1H, s) 7.50-7.45 (1H, m) 7.42-7.35 (1H, m) 7.28 |
| | (1H, s) 7.16 (1H, dd, J = 7.8, 1.4 Hz) 6.21 (2H, s) 5.29 (2H, s) |
| 5 | 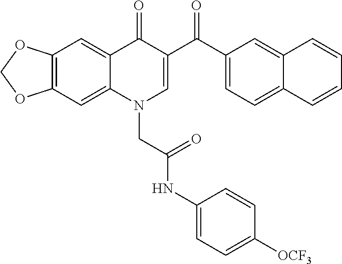 [SMILES: O=C(Cn1cc(C(=O)c2ccc3ccccc3c2)c(=O)c2cc3c(cc21)OCO3)Nc1ccc(OC(F)(F)F)cc1] |
| | 2-[6,7-Methylenedioxy-3-(2-naphthoyl)-4-oxo-1-quinolinyl]-N- |
| | (4-trifluoromethoxyphenyl)acetamide |
| | 10.69 (1H, s) 8.48 (1H, s) 8.32 (1H, s) 8.05 (1H, d, J = 8.2 Hz) |
| | 7.97-7.94 (2H, m) 7.81 (1H, dd, J = 8.6, 1.6 Hz) 7.75-7.69 (2H, m) |
| | 7.68-7.62 (1H, m) 7.61-7.55 (1H, m) 7.54 (1H, s) 7.36 (2H, d, |
| | J = 8.6 Hz) 7.27 (1H, s) 6.21 (2H, s) 5.29 (2H, s) |
| 6 | 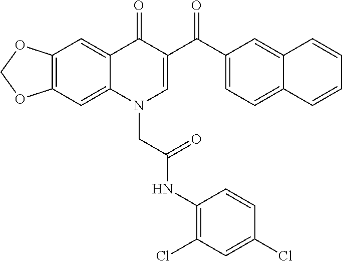 [SMILES: O=C(Cn1cc(C(=O)c2ccc3ccccc3c2)c(=O)c2cc3c(cc21)OCO3)Nc1ccc(Cl)cc1Cl] |
| | N-(2,4-DichlorophenyI)-2-[6,7-methylenedioxy-3-(2-naphthoyl)- |
| | 4-oxo-1-quinolinyl]acetamide |
| | 10.23 (1H, s) 8.46 (1H, s) 8.33-8.30 (1H, m) 8.04 (1H, d, J = 8.2 |
| | Hz) 7.97-7.93 (2H, m) 7.84-7.77 (2H, m) 7.72 (1H, d, J = 2.4 Hz) |
| | 7.68-7.61 (1H, m) 7.61-7.55 (1H, m) 7.53 (1H, s) 7.44 (1H, dd, |
| | J = 8.6, 2.4 Hz) 7.26 (1H, s) 6.22 (2H, s) 5.39 (2H, s) |
| 7 | 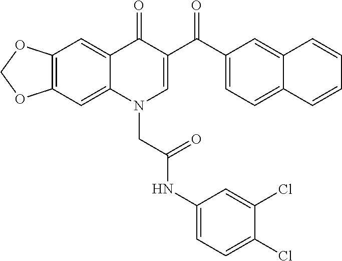 [SMILES: O=C(Cn1cc(C(=O)c2ccc3ccccc3c2)c(=O)c2cc3c(cc21)OCO3)Nc1ccc(Cl)c(Cl)c1] |
| | N-(3,4-Dichlorophenyl)-2-[6,7-methylenedioxy-3-(2-naphthoyl)- |
| | 4-oxo-1-quinolinyl]acetamide |
| | 10.78 (1H, s) 8.47 (1H, s) 8.33-8.31 (1H, m) 8.05 (1H, d, J = 8.2 |
| | Hz) 8.01-7.96 (3H, m) 7.81 (1H, dd, J = 8.4, 1.2 Hz) 7.68-7.55 |
| | (3H, m) 7.54-7.49 (2H, m) 7.29 (1H, s) 6.21 (2H, s) 5.30 (2H, s) |
| 8 | 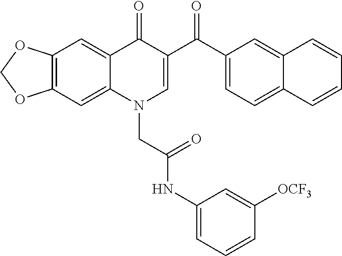 [SMILES: O=C(Cn1cc(C(=O)c2ccc3ccccc3c2)c(=O)c2cc3c(cc21)OCO3)Nc1cccc(OC(F)(F)F)c1] |
| | 2-[6,7-Methylenedioxy-3-(2-naphthoyI)-4-oxo-1-quinolinyl]-N- |
| | (3-trifluoromethoxyphenyl)acetamide |
| | 10.82 (1H, s) 8.47 (1H, s) 8.32 (1H, s) 8.05 (1H, d, J = 8.2 Hz) |
| | 7.97-7.94 (2H, m) 7.82 (1H, dd, J = 8.6, 1.6 Hz) 7.80-7.77 (1H, m) |
| | 7.68-7.61 (1H, m) 7.60-7.55 (1H, m) 7.54 (1H, s) 7.53-7.45 (2H, |
| | m) 7.31 (1H, s) 7.09 (1H, d, J = 8.0 Hz) 6.21 (2H, s) 5.30 (2H, s) |
| 9 | 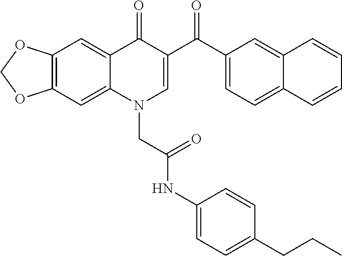 [SMILES: CCCc1ccc(NC(=O)Cn2cc(C(=O)c3ccc4ccccc4c3)c(=O)c3cc4c(cc32)OCO4)cc1] |
| | 2-[6,7-Methylenedioxy-3-(2-naphthoyl)-4-oxo-1-quinolinyl]-N- |
| | (4-propylphenyl)acetamide |
| | 10.41 (1H, s) 8.47 (1H, s) 8.33-8.31 (1H, m) 8.05 (1H, d, J = 8.0 |
| | Hz) 7.97-7.94 (2H, m) 7.81 (1H, dd, J = 8.6, 1.6 Hz) 7.68-7.62 |
| | (1H, m) 7.61-7.55 (1H, m) 7.53-7.48 (3H, m) 7.24 (1H, s) |
| | 7.18-7.13 (2H, m) 6.20 (2H, s) 5.25 (2H, s) 2.53-2.51 (2H, m, |
| | overlapped with DMSO) 1.61-1.50 (2H, m) 0.87 (3H, t, J = 7.4 |
| | Hz) |
| 10 | 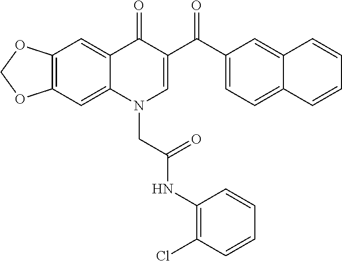 [SMILES: O=C(Cn1cc(C(=O)c2ccc3ccccc3c2)c(=O)c2cc3c(cc21)OCO3)Nc1ccccc1Cl] |
| | N-(2-ChlorophenyI)-2-[6,7-methylenedioxy-3-(2-naphthoyl)-4- |
| | oxo-1-quinolinyl]acetamide |
| | 10.16 (1H, s) 8.47 (1H, s) 8.32 (1H, s) 8.04 (1H, d, J = 8.2 Hz) |
| | 8.00-7.97 (2H, m) 7.82 (1H, d, J = 8.8 Hz) 7.75 (1H, d, J = 8.0 Hz) |
| | 7.68-7.61 (1H, m) 7.60-7.52 (3H, m) 7.39-7.31 (1H, m) 7.30-7.20 |
| | (2H, m) 6.23 (2H, s) 5.39 (2H, s) |
| 11 | 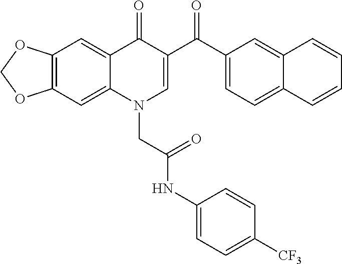 [SMILES: O=C(Cn1cc(C(=O)c2ccc3ccccc3c2)c(=O)c2cc3c(cc21)OCO3)Nc1ccc(C(F)(F)F)cc1] |
| | 2-[6,7-Methylenedioxy-3-(2-naphthoyl)-4-oxo-1-quinolinyl]-N- |
| | (4-trifluoromethylphenyl)acetamide |
| | 10.81 (1H, s) 8.45 (1H, s) 8.29 (1H, s) 8.02 (1H, d, J = 8.0 Hz) |
| | 7.95 (2H, dd, J = 8.2, 4.0 Hz) 7.80-7.77 (3H, m) 7.70 (1H, s) 7.67 |
| | (1H, s) 7.63-7.59 (1H, m) 7.56-7.52 (1H, m) 7.50 (1H, s) 7.27 |
| | (1H, s) 6.17 (2H, s) 5.29 (2H, s) |
| 12 | 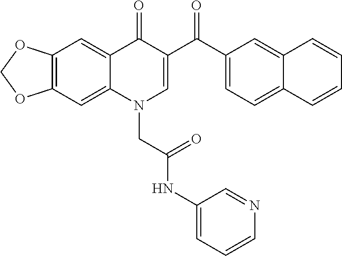 [SMILES: O=C(Cn1cc(C(=O)c2ccc3ccccc3c2)c(=O)c2cc3c(cc21)OCO3)Nc1cccnc1] |
| | 2-[6,7-Methylenedioxy-3-(2-naphthoyl)-4-oxo-1-quinolinyl]-N- |
| | (3-pyridyl)acetamide |
| | 11.01 (1H, s) 8.90 (1H, s) 8.44 (1H, d, J = 1.8 Hz) 8.40-8.38 (1H, |
| | m) 8.29 (1H, s) 8.17 (1H, d, J = 8.5) 8.02 (1H, d, J = 7.9 Hz) |
| | 7.97-7.94 (2H, m) 7.78 (1H, d, J = 8.8 Hz) 7.63-7.53 (3H, m) 7.50 |
| | (1H, s) 7.28 (1H, s) 6.17 (2H, s) 5.33 (2H, s) |
| 13 | 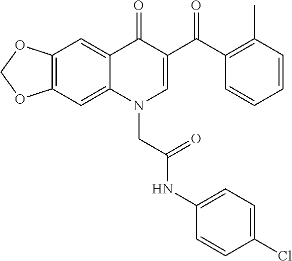 [SMILES: Cc1ccccc1C(=O)c1cn(CC(=O)Nc2ccc(Cl)cc2)c2cc3c(cc2c1=O)OCO3] |
| | N-(4-Chlorophenyl)-2-(6,7-methylenedioxy-3-o-toluoyl-4-oxo-1- |
| | quinolinyl)acetamid |
| | 2.31 (s, 3H); 5.28 (s, 2H); 6.18 (s, 2H); 7.15-7.27 (m, 4H); |
| | 7.30-7.37 (m, 1H); 7.37-7.42 (m, 2H); 7.46 (s, 1H); 7.60-7.66 (m, |
| | 2H); 8.50 (s, 1H); 10.63 (s, 1H) |
| 14 | 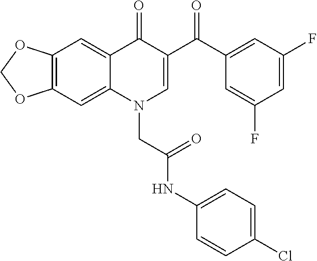 [SMILES: O=C(Cn1cc(C(=O)c2cc(F)cc(F)c2)c(=O)c2cc3c(cc21)OCO3)Nc1ccc(Cl)cc1] |
| | N-(4-Chlorophenyl)-2-[3-(3,5-difluorobenzoyl)-6,7-methylene- |
| | dioxy-4-oxo-1-quinolinyl]acetamide |
| | 5.28 (s, 2H); 6.20 (s, 2H); 7.27 (s, 1H); 7.30-7.36 (m, 2H) |
| | 7.37-7.42 (m, 2H); 7.49 (tt, J = 2.4, 9.2 Hz, 1H); 7.53 (s, 1H); |
| | 7.60-7.65 (m, 2H); 8.50 (s, 1H); 10.61 (s, 1H) |
| 15 | 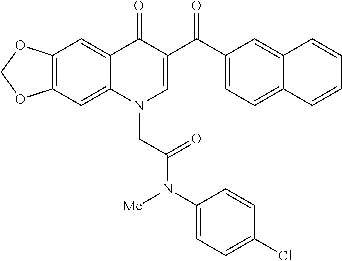 [SMILES: CN(C(=O)Cn1cc(C(=O)c2ccc3ccccc3c2)c(=O)c2cc3c(cc21)OCO3)c1ccc(Cl)cc1] |
| | N-(4-Chlorophenyl)-N-methyl-2-[6,7-methylenedioxy-3-(2- |
| | naphthoyl)-4-oxo-1-quinolinyl]acetamide |
| | 8.27 (2H, d, J = 5.3 Hz) 8.03-7.92 (3H, m) 7.77-7.36 (8H, m) 7.05 |
| | (1H, s) 6.19 (2H, s) 5.55 (1H, s) 4.95 (1H, s) 3.19 (3H, s) |

### Examples 16-21

The title compounds where prepared in accordance with Example 1, using the appropriately substituted aroylchloride in step (a), see Table 2.

TABLE 2

| | Chemical structure |
|---|---|
| | Name |
| Example | ¹H NMR (400 MHz, DMSO-d₆) δ |
| 16 | 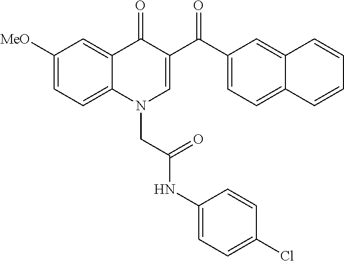 [SMILES: COc1ccc2c(c1)c(=O)c(C(=O)c1ccc3ccccc3c1)cn2CC(=O)Nc1ccc(Cl)cc1] N-(4-Chlorophenyl)-2-[6-methoxy-3-(2-naphthoyl)-4-oxo-1- quinolinyl]acetamide 3.85 (s, 3H); 5.34 (s, 2H); 7.40 (AA′BB′ mult., J = 8.8 Hz, 2H); 7.44 (dd, J = 3.1, 9.3 Hz, 1H); 7.58 (ddd, J = 1.1, 7.0, 8.1 Hz, 1H); 7.63 (AA′BB′ mult., J = 8.8 Hz, 2H); 7.60-7.67 (m, 3H); 7.82 (dd, J = 1.7, 8.6 Hz, 1H); 7.98 (d, J = 8.6 Hz, 1H); 7.99 (d, J = 8.1 Hz, 1H); 8.06 (d, J = 8.1 Hz, 1H); 8.34 (s, 1H); 8.55 (s, 1H); 10.70 (s, 1H) |
| 17 | 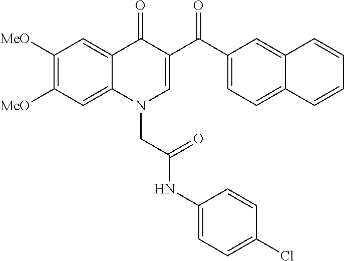 [SMILES: COc1cc2c(=O)c(C(=O)c3ccc4ccccc4c3)cn(CC(=O)Nc3ccc(Cl)cc3)c2cc1OC] N-(4-Chlorophenyl)-2-[6,7-dimethoxy-3-(2-naphthoyl)-4-oxo-1- quinolinyl]acetamide 3.85 (s, 3H); 3.87 (s, 3H); 5.33 (s, 2H); 7.04 (s, 1H); 7.40 (AA′BB′ mult., J = 8.8 Hz, 2H); 7.58 (ddd, J = 1.2, 7.0, 8.1 Hz, 1H); 7.60 (s, 1H); 7.63 (AA′BB′ mult., J = 8.8 Hz, 2H); 7.64 (ddd, J = 1.2, 7.0, 8.1 Hz, 1H); 7.81 (dd, J = 1.7, 8.6 Hz, 1H); 7.98 (d, J = 8.6 Hz, 1H); 7.99 (d, J = 8.1 Hz, 1H); 8.05 (d, J = 8.1 Hz, 1H); 8.33 (s, 1H); 8.49 (s, 1H); 10.72 (s, 1H) |
| 18 | 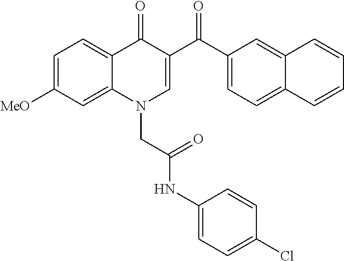 [SMILES: COc1ccc2c(=O)c(C(=O)c3ccc4ccccc4c3)cn(CC(=O)Nc3ccc(Cl)cc3)c2c1] N-(4-Chlorophenyl)-2-[7-methoxy-3-(2-naphthoyl)-4-oxo-1- quinolinyl]acetamide 3.87 (s, 3H); 5.29 (s, 2H); 7.00 (d, J = 2.1 Hz, 1H); 7.12 (dd, J = 2.1, 8.9 Hz, 1H); 7.40 (AA′BB′ mult., J = 8.8 Hz, 2H); 7.58 (ddd, J = 1.1, 7.0, 8.1 Hz, 1H); 7.63 (AA′BB′ mult., J = 8.8 Hz, 2H); 7.65 (m, 1H); 7.82 (dd, J = 1.6, 8.6 Hz, 1H); 7.98 (d, J = 8.6 Hz, 1H); 7.99 (d, J = 8.1 Hz, 1H); 8.06 (d, J = 8.1 Hz, 1H); 8.15 (d, J = 8.9 Hz, 1H); 8.33 (s, 1H); 8.52 (s, 1H); 10.70 (s, 1H) |
| 19 | 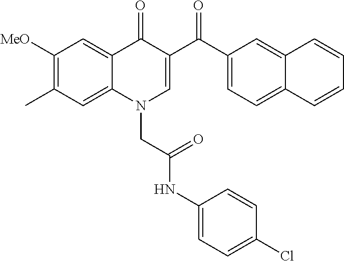 [SMILES: COc1cc2c(=O)c(C(=O)c3ccc4ccccc4c3)cn(CC(=O)Nc3ccc(Cl)cc3)c2cc1C] N-(4-Chlorophenyl)-2-[6-methoxy-7-methyl-3-(2-naphthoyl)- 4-oxo-1-quinolinyl]acetamide 2.32 (s, 3H); 3.88 (s, 3H); 5.31 (s, 2H); 7.40 (AA′BB′ mult., J = 8.8 Hz, 2H); 7.54 (s, 1H); 7.57 (ddd, J = 1.2, 7.0, 8.1 Hz, 1H); 7.60 (s, 1H); 7.64 (AA′BB′ mult., J = 8.8 Hz, 2H); 7.64 (ddd, J = 1.3, 7.0, 8.1 Hz, 1H); 7.81 (dd, J = 1.6, 8.5 Hz, 1H); 7.97 (d, J = 8.5 Hz, 1H); 7.99 (d, J = 8.1 Hz, 1H); 8.05 (d, J = 8.1 Hz, 1H); 8.33 (s, 1H); 8.51 (s, 1H); 10.65 (s, 1H) |
| 20 | 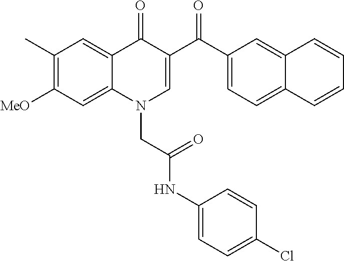 [SMILES: COc1cc2c(cc1C)c(=O)c(C(=O)c1ccc3ccccc3c1)cn2CC(=O)Nc1ccc(Cl)cc1] N-(4-Chlorophenyl)-2-[7-methoxy-6-methyl-3-(2-naphthoyl)-4- oxo-1-quinolinyl]acetamide 2.24 (s, 3H); 3.88 (s, 3H); 5.31 (s, 2H); 6.95 (s, 1H); 7.40 (AA′BB′ mult., J = 8.8 Hz, 2H); 7.57 (ddd, J = 1.2, 7.0, 8.1 Hz, 1H); 7.62 (AA′BB′ mult., J = 8.8 Hz, 2H); 7.64 (ddd, J = 1.3, 7.0, 8.1 Hz, 1H); 7.82 (dd, J = 1.6, 8.6 Hz, 1H); 7.97 (d, J = 8.6 Hz, 1H); 7.97 (m, 1H); 7.99 (d, J = 8.1 Hz, 1H); 8.05 (d, J = 8.1 Hz, 1H); 8.33 (s, 1H); 8.50 (s, 1H); 10.74 (s, 1H) |
| 21 | 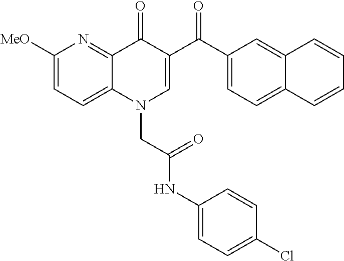 [SMILES: COc1ccc2c(n1)c(=O)c(C(=O)c1ccc3ccccc3c1)cn2CC(=O)Nc1ccc(Cl)cc1] N-(4-Chlorophenyl)-2-[6-methoxy-3-(2-naphthoyl)-4-oxo- (1,5-naphthyridin-1-yl)]acetamide 3.95 (s, 3H); 5.34 (s, 2H); 7.29 (d, J = 9 3 Hz, 1H); 7.40 (AA′BB′ mult., J = 8.8 Hz, 2H); 7.58 (ddd, J = 1.1, 7.0, 8.0 Hz, 1H); 7.63 (AA′BB′ mult., J = 8.8 Hz, 2H); 7.65 (ddd, J = 1.1, 7.0, 8.0 Hz, 1H); 7.84 (dd, J = 16, 8.6 Hz, 1H); 8.00 (two overlapped d, J = 8.0 and 8.6 Hz, 2H); 8.07 (d, J = 8.0 Hz, 1H); 8.12 (d, J = 9.3 Hz, 1H); 8.35 (s, 1H); 8.53 (s, 1H); 10.66 (s, 1H) |

### Example 22

### N-(4-Chlorophenyl)-2-[6-hydroxy-3-(2-naphthoyl)-4-oxo-(1,5-naphthyridin-1-yl)]-acetamide

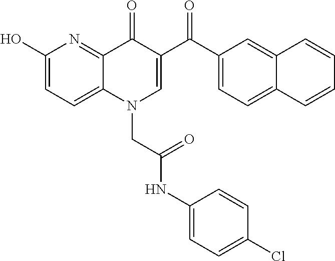 [SMILES: O=C(Cn1cc(C(=O)c2ccc3ccccc3c2)c(=O)c2nc(O)ccc21)Nc1ccc(Cl)cc1]

A mixture of N-(3-chlorophenyl)-2-[6-methoxy-3-(2-naphthoyl)-4-oxo-(1,5-naph-thyridin-1-yl)]acetamide (87 mg, 0.17 mmol, see Example 21), LiCl (37 mg, 0.87 mmol), p-toluenesulfonic acid monohydrate (166 mg, 0.87 mmol) and DMF (4 mL) was heated at 120° C. for 3 h and concentrated. The residue was treated with EtOH/H₂O (1:1) (8 mL) and the solid formed was collected, washed with EtOH and dried to give the title compound (48 mg, 57%).

¹H NMR (400 MHz, DMSO-d₆) δ: 5.34 (s, 2H); 6.84 (d, J=10.1 Hz, 1H); 7.40 (AA′BB′ mult., J=8.8 Hz, 2H); 7.58 (ddd, J=1.1, 6.9, 8.0 Hz, 1H); 7.63 (AA′BB′ mult., J=8.8 Hz, 2H); 7.66 (ddd, J=1.2, 6.9, 8.1 Hz, 1H); 7.85 (dd, J=1.7, 8.6 Hz, 1H); 7.96 (d, J=10.1 Hz, 1H); 8.00 (d, J=8.0 Hz, 1H); 8.00 (d, J=8.6 Hz, 1H); 8.07 (d, J=8.1 Hz, 1H); 8.37 (unresolved d, J˜1.7 Hz, 1H); 8.46 (s, 1H); 10.65 (s, 1H); 11.00 (b s, 1H)

### Example 23

### N-(4-Chlorophenyl)-2-[7-methoxy-3-(2-naphthoyl)-4-oxo-(1,6-naphthyridin-1-yl)]-acetamide

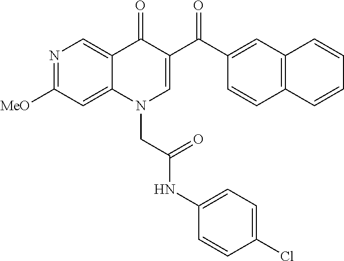 [SMILES: COc1cc2c(cn1)c(=O)c(C(=O)c1ccc3ccccc3c1)cn2CC(=O)Nc1ccc(Cl)cc1]

### (a) 2-(4,6-Dichloronicotinoyl)-3-(dimethylamino)acrylic acid ethyl ester

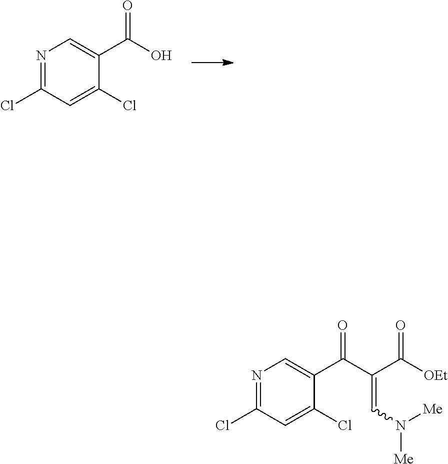 [SMILES: O=C(O)c1cnc(Cl)cc1Cl ; CCOC(=O)C(=CN(C)C)C(=O)c1cnc(Cl)cc1Cl]

A mixture of 4,6-dichloronicotinic acid (0.8 g, 3.9 mmoles) and thionyl chloride (5 ml) was heated at reflux for 2 h. The mixture was concentrated and the residue was dissolved in toluene (10 ml) and added to ethyl 3-(dimethylamino)acrylate (0.84 g, 5.85 mmoles) and triethyl amine (0.6 g, 5.85 mmoles). The mixture was heated at 90° C. for 4 h, cooled, filtered and concentrated. Purification by chromatography gave the sub-tile compound (0.7 g, 54%).

### (b) 7-Methoxy-1-tert-octyl-4-oxo-1,6-naphthyridine-3-carboxylic acid

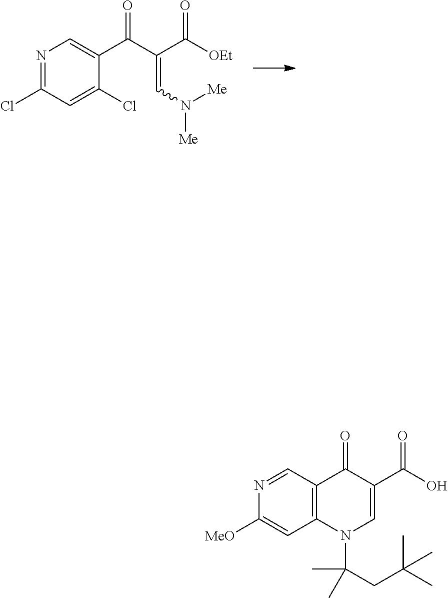 [SMILES: CCOC(=O)C(=CN(C)C)C(=O)c1cnc(Cl)cc1Cl ; COc1cc2c(cn1)c(=O)c(C(=O)O)cn2C(C)(C)CC(C)(C)C]

tert-Octylamine (464 mg, 3.6 mmol) was added to a mixture of 2-(4,6-dichloro-nicotinoyl)-3-(dimethylamino)acrylic acid ethyl ester (1046 mg, 3.3 mmol) and MeOH. After 3 h at it NaOMe in MeOH (1 M, 33 mmol) was added and the mixture was heated at reflux for 4 h. The mixture was allowed to cool and concentrated. Water (˜2 mL) was added and the pH was adjusted to ˜1 with 2 M HCl. The mixture was stirred overnight and the precipitate was filtered off, washed with water and dried to give the sub-title compound (844 mg, 77%).

### (c) N,7-Dimethoxy-N-methyl-1-tert-octyl-4-oxo-1,6-naphthyridine-3-carboxamide

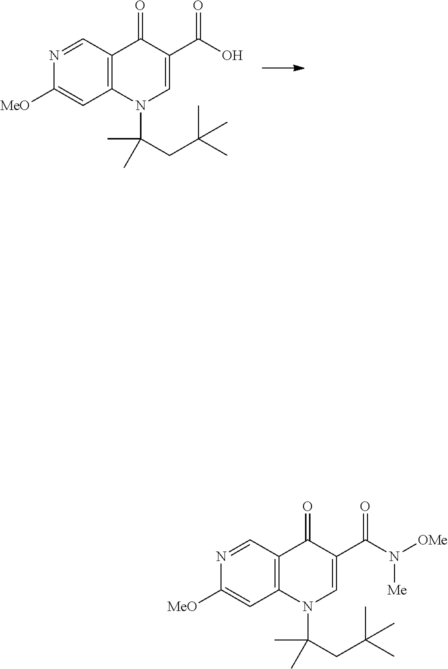 [SMILES: COc1cc2c(cn1)c(=O)c(C(=O)O)cn2C(C)(C)CC(C)(C)C ; COc1cc2c(cn1)c(=O)c(C(=O)N(C)OC)cn2C(C)(C)CC(C)(C)C]

Triethylamine (0.8 mL, 6.3 mmol) and isobutyl chloroformate (0.38 mL, 2.9 mmol) were added to a mixture of 7-methoxy-1-tert-octyl-4-oxo-1,6-naphthyridine-3-carboxylic acid (840 mg, 2.5 mmol) and CH₂Cl₂ (15 mL) at 0° C. The mixture was stirred at 0° C. for 30 min and N,O-dimethylhydroxylamine hydrochloride (296 mg, 3.0 mmol) was added. The mixture was stirred at it for 1 h and diluted with chloroform and NH₄Cl (aq., sat). The layers were separated and the organic layer was washed with NaCl (aq., sat), dried (Na₂SO₄) and concentrated. Purification by chromatography gave the sub-title compound (884 mg, 93%).

### (d) 7-Metoxy-3-(2-naphthoyl)-1-tert-octyl-4-oxo-1,6-naphthyridine

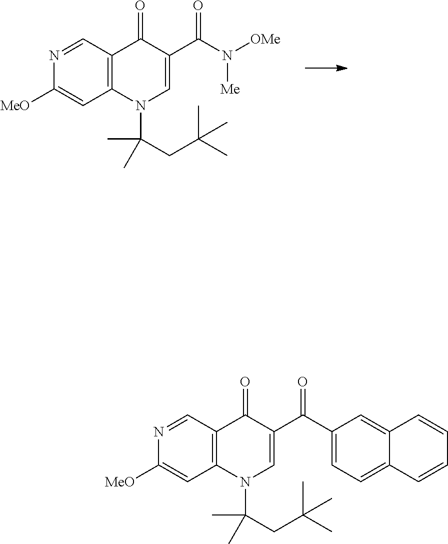 [SMILES: COc1cc2c(cn1)c(=O)c(C(=O)N(C)OC)cn2C(C)(C)CC(C)(C)C ; COc1cc2c(cn1)c(=O)c(C(=O)c1ccc3ccccc3c1)cn2C(C)(C)CC(C)(C)C]

2-Naphthylmagnesium bromide (0.5 M in THF, 1.4 mL, 0.70 mmol) was added to N,7-dimethoxy-N-methyl-1-tert-octyl-4-oxo-1,6-naphthyridine-3-carboxamide (175 mg, 0.47 mmol) in THF (2 mL) at rt. The mixture was stirred at rt for 3 h. NH₄Cl (aq., sat) was added and the mixture was extracted with CH₂Cl₂. The combined extracts were dried (Na₂SO₄) and concentrated. Purification by chromatography gave the sub-title compound (49 mg, 24%).

### (e) 7-Methoxy-3-(2-naphthoyl)-4-oxo-1,6-naphthyridine

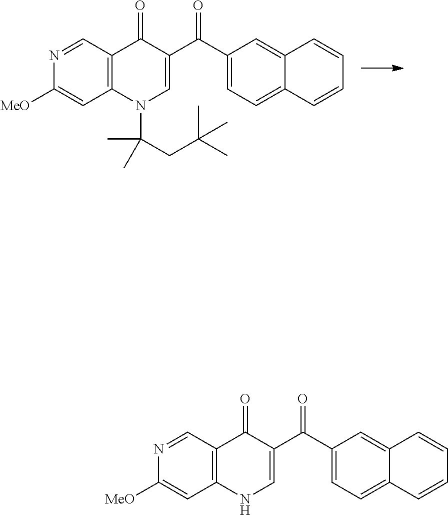 [SMILES: COc1cc2c(cn1)c(=O)c(C(=O)c1ccc3ccccc3c1)cn2C(C)(C)CC(C)(C)C ; COc1cc2[nH]cc(C(=O)c3ccc4ccccc4c3)c(=O)c2cn1]

A mixture of 7-metoxy-3-(2-naphthoyl)-1-tert-octyl-4-oxo-1,6-naphthyridine, TFA (2 mL) and CH₂Cl₂ (2 mL) was stirred at rt for 30 min. Concentration gave the sub-title compound (32 mg, 95%).

### (f) N-(4-Chlorophenyl)-2-[7-methoxy-3-(2-naphthoyl)-4-oxo-(1,6-naphthyridin-1-yl)]acetamide

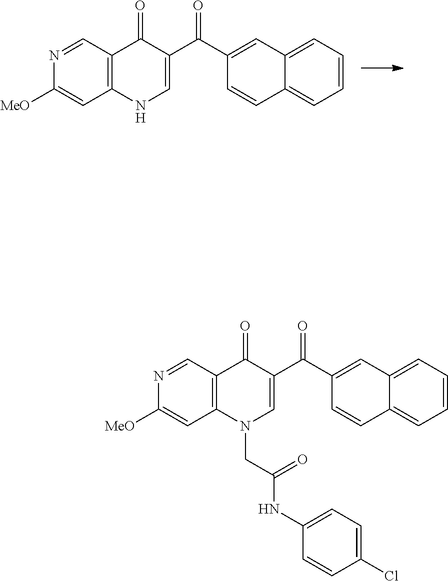 [SMILES: COc1cc2[nH]cc(C(=O)c3ccc4ccccc4c3)c(=O)c2cn1 ; COc1cc2c(cn1)c(=O)c(C(=O)c1ccc3ccccc3c1)cn2CC(=O)Nc1ccc(Cl)cc1]

2-Chloro-N-(4-chlorophenyl)acetamide (22 mg, 0.11 mmol, see Example 1, Step (e)) was added to a mixture of 7-methoxy-3-(2-naphthoyl)-4-oxo-1,6-naphthyridine (32 mg, 0.97 mmol), K₂CO₃ (33 mg, 0.24 mmol) and DMF (2 mL). The mixture was stirred at it for 72 h, filtered and concentrated. The residue was washed with EtOH (3 mL) and crystallysed from DMF (˜3 mL), EtOH (˜2 mL) and H₂O (1 drop) to give the title compound (16 mg, 34%).

¹H NMR (400 MHz, DMSO-d₆) δ: 2.25 (s, 3H); 2.28 (s, 3H); 3.74 (s, 3H); 3.86 (s, 3H); 5.26 (s, 2H); 6.20 (s, 2H); 6.48 (dd, J=2.5, 8.8 Hz, 1H); 6.65 (d, J=2.5 Hz, 1H); 7.21 (s, 1H); 7.22 (d, J=7.9 Hz, 1H); 7.45 (dd, J=1.5, 7.9 Hz, 1H); 7.52 (s, 1H); 7.52 (s, 1H); 7.68 (d, J=8.8 Hz, 1H); 8.28 (s, 1H); 9.67 (s, 1H)

### Example 24

### N-(4-Chlorophenyl)-2-[6-methoxy-3-(2-naphthoyl)-4-oxo-(1,7-naphthyridin-1-yl)]-acetamide

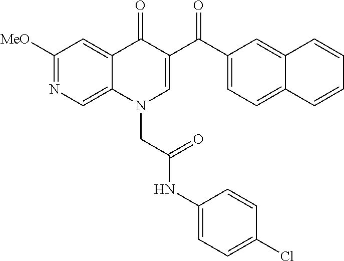 [SMILES: COc1cc2c(=O)c(C(=O)c3ccc4ccccc4c3)cn(CC(=O)Nc3ccc(Cl)cc3)c2cn1]

### (a) 8-Chloro-6-methoxy-3-(2-naphthoyl)-4-oxo-1,7-naphthyridine

The sub-title compound was prepared in accordance with Example 1, Steps (a) to (d) using 3-amino-2-chloro-6-methoxypyridine in Step (c).

### (b) 6-Methoxy-3-(2-naphthoyl)-4-oxo-1,7-naphthyridine

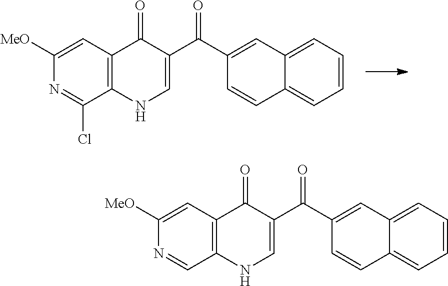 [SMILES: COc1cc2c(=O)c(C(=O)c3ccc4ccccc4c3)c[nH]c2c(Cl)n1 ; COc1cc2c(=O)c(C(=O)c3ccc4ccccc4c3)c[nH]c2cn1]

A mixture of 8-Chloro-6-methoxy-3-(2-naphthoyl)-4-oxo-1,7-naphthyridine (54 mg, 0.15 mmol), Pd/C (10%, 7.9 mg, 0.07 mmol), Et₃N (0.02 mL, 0.15 mmol), EtOH (5 mL), EtOAc (6 mL) and MeOH (5 mL) was hydrogenated at 3 atm and rt for 8 h. The mixture was filtered and the filtrate treated with CH₂Cl₂/EtOAc (1:1). The precipitate formed was collected and purified by chromatography to give the sub-title compound (37 mg, 76%).

### (c) N-(4-Chlorophenyl)-2-[6-methoxy-3-(2-naphthoyl)-4-oxo-(1,7-naphthyridin-1-yl)]acetamide

The title compound was prepared in accordance with Example 1, Step (e).

¹H NMR (400 MHz, DMSO-d₆) δ: 3.94 (s, 3H); 5.42 (s, 2H); 7.38 (s, 1H); 7.40 (AA′BB′ mult., J=8.9 Hz, 2H); 7.58 (ddd, J=1.2, 7.0, 8.0 Hz, 1H); 7.61-7.68 (m, 3H); 7.82 (dd, J=1.6, 8.6 Hz, 1H); 7.99 (d, J=8.6 Hz, 1H); 8.00 (d, J=8.0 Hz, 1H); 8.05 (d, J=8.0 Hz, 1H); 8.35 (s, 1H); 8.62 (s, 1H); 8.84 (s, 1H); 10.70 (s, 1H)

### Example 25

### N-(4-Chlorophenyl)-2-[3-(4-fluorobenzoyl-5-methoxy-4-oxo-1-quinolinyl]acetamide

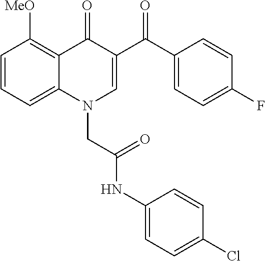 [SMILES: COc1cccc2c1c(=O)c(C(=O)c1ccc(F)cc1)cn2CC(=O)Nc1ccc(Cl)cc1]

### (a) 3-(2-Bromo-5-methoxyphenylamino)-2-(4-fluorobenzoyl)acrylic acid methyl ester

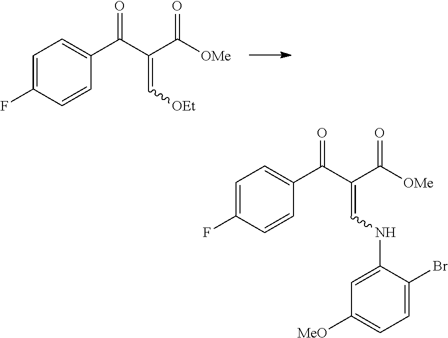 [SMILES: CCOC=C(C(=O)OC)C(=O)c1ccc(F)cc1 ; COC(=O)C(=CNc1cc(OC)ccc1Br)C(=O)c1ccc(F)cc1]

The sub-title compound was prepared in accordance with Example 1, Step (c) from 3-ethoxy-2-(4-fluorobenzoyl)acrylic acid methyl ester and 2-bromo-5-methoxyaniline.

### (b) 8-Bromo-3-(4-fluorobenzoyl)-5-methoxy-4-oxoquinoline

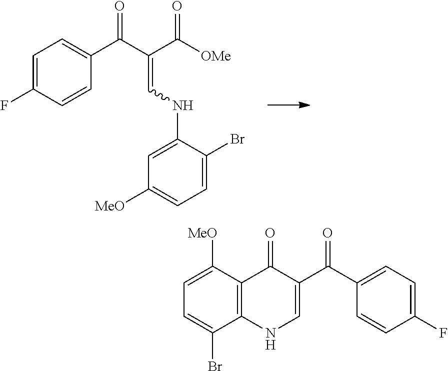

3-(2-Bromo-5-methoxyphenylamino)-2-(4-fluorobenzoyl)acrylic acid methyl ester (242 mg, 0.59 mmol) was added carefully with stirring to polyphosphoric acid (3 g) at 140° C. The mixture was stirred for 30 min at 140° C. and allowed to cool to rt. Absolute EtOH (4 mL) was added and the mixture was heated at reflux for 45 min, allowed to cool to rt, neutralized with aqueous NaHCO₃ (aq., sat) and extracted with CH₂Cl₂. The extracts were combined, washed with brine and concentrated. The residue was crystallized from MeOH giving the sub-title compound (52 mg, 23%).

### (c) 3-(4-Fluorobenzoyl)-5-methoxy-4-oxoquinoline

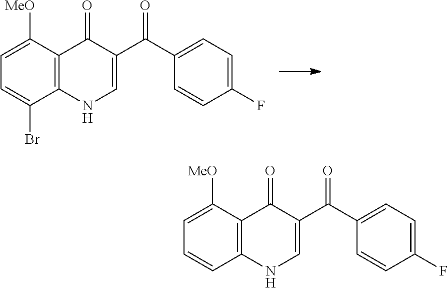 [SMILES: COc1ccc(Br)c2[nH]cc(C(=O)c3ccc(F)cc3)c(=O)c12 ; COc1cccc2[nH]cc(C(=O)c3ccc(F)cc3)c(=O)c12]

A mixture of 8-bromo-3-(4-fluorobenzoyl)-5-methoxy-4-oxoquinoline (50 mg, 0.13 mmol), Pd—C (10%, 7.1 mg), NaOH (aq., 2.5 M, 0.11 mL, 0.27 mmol), MeOH (5 mL), EtOH (2 mL) and EtOAc (5 mL) was hydrogenated at rt and 3.5 atm for 2 h. The mixture was filtered, concentrated, treated with NaOH (aq., 2.5 M), washed with CH₂Cl₂ and filtered. Acidification to pH˜3 with 2 M HCl gave a precipitate which was collected, washed with water and dried to give the sub-title compound (12 mg, 30%).

### (d) N-(4-Chlorophenyl)-2-[3-(4-fluorobenzoyl-5-methoxy-4-oxo-1-quinolinyl]-acetamide

The title compound was prepared from 3-(4-fluorobenzoyl)-5-methoxy-4-oxoquinoline and 2-chloro-N-(4-chlorophenyl)acetamide in accordance with Example 1, Step f. ¹H NMR (400 MHz, DMSO-d₆) δ: 3.80 (s, 3H); 5.20 (s, 2H); 6.96 (d, J=8.4 Hz, 1H); 7.03 (d, J=8.5 Hz, 1H); 7.30 (t, J=8.9 Hz, 2H); 7.39 (AA′BB′ mult., J=8.9 Hz, 2H); 7.62 (AA′BB′ mult., J=8.9 Hz, 2H); 7.65 (t, J=8.5 Hz, 1H); 7.79 (dd, J=5.6, 8.9 Hz, 2H); 8.33 (s, 1H); 10.67 (s, 1H)

### Example 26

### N-p-Anisyl-2-[3-(2-furoyl)-6,7-methylenedioxy-4-oxo-1-quinolinyl]acetamide

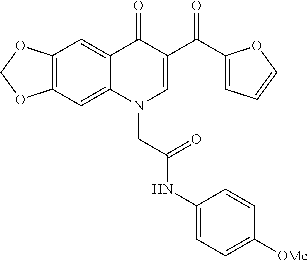 [SMILES: COc1ccc(NC(=O)Cn2cc(C(=O)c3ccco3)c(=O)c3cc4c(cc32)OCO4)cc1]

### (a) 2-Furoylacetic acid ethyl ester

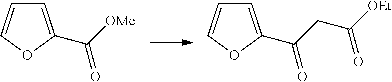 [SMILES: COC(=O)c1ccco1 ; CCOC(=O)CC(=O)c1ccco1]

Furoic acid methyl ester (22.7 g, 0.18 mol) was heated to 80° C. Na (2 g, 87 mmol) was added in small portions followed by dropwise addition of EtOAc (0.1 mol, 8.8 g). After complete dissolution of the Na, the mixture was heated to 90-95° C. and additional amounts of Na (2 g) and EtOAc (8.8 g) were added. After complete dissolution of the Na, benzene (100 mL) and four additional portions of Na (4×2 g) and EtOAc (4×8.8 g) were added. (The total amounts of Na and EtOAc were 12 g, 0.52 mol and 52.8 g, 0.6 mol respectively). The mixture was heated at 90-95° C. for 10 h, ice-water (60 mL) was added and the mixture was diluted with HCl (5%, aq., 250 mL). The layers were separated and the aqueous layer was extracted with Et₂O (2×100 mL). The combined extracts were dried (Na₂SO₄) and concentrated to give the sub-title compound (14.1 g, 43%) which was ca 82% pure and was used in the next step without further purification.

### (b) 3-Ethoxy-2-(2-furoyl)acrylic acid ethyl ester

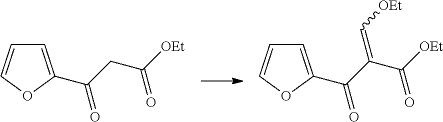 [SMILES: CCOC(=O)CC(=O)c1ccco1 ; CCOC=C(C(=O)OCC)C(=O)c1ccco1]

The sub-title compound was prepared from 2-furoylacetic acid ethyl ester in accordance with Example 1, Step (b).

### (c) 2-(2-Furoyl)-3-(3,4-methylenedioxyphenylamino)acrylic acid ethyl ester

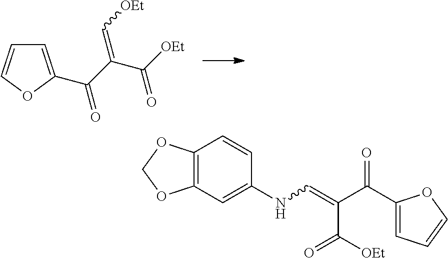

3,4-Methylenedioxyaniline (6.6 g, 0.05 mol) in absolute EtOH (50 mL) was added drop-wise to a stirred mixture of 3-ethoxy-2-(2-furoyl)acrylic acid ethyl ester (11.9 g, 0.05 mol) and absolute EtOH (100 mL). The mixture was heated at 60° C. for 8 h. The mixture was concentrated and the residue purified by chromatography to give the sub-title compound which was used in the next step without further purification.

### (d) N-p-Anisyl-2-[3-(2-furoyl)-6,7-methylenedioxy-4-oxo-1-quinolinyl]-acetamide

The title compound was prepared in accordance with Example 1, Steps (d) and (f). The reaction time in step (d) was 2 h. The total yield starting from furoic acid methyl ester was 13%.

¹H NMR (400 MHz, DMSO-d₆) δ 10.34 (s, 1H), 8.37 (s, 1H), 7.98 (s, 1H), 7.57 (s, 1H), 7.51 (d, J=8.6 Hz, 2H), 7.32 (d, J=3.6 Hz, 1H), 7.21 (s, 1H), 6.90 (d, J=8.6 Hz, 2H), 6.74-6.68 (m, 1H), 6.20 (s, 2H), 5.18 (s, 2H), 3.72 (s, 3H).

### Example 27

### N-(5-chloro-2-pyridyl)-2-[3-(2-furoyl)-6,7-methylenedioxy-4-oxo-1-quinolinyl]-acetamide

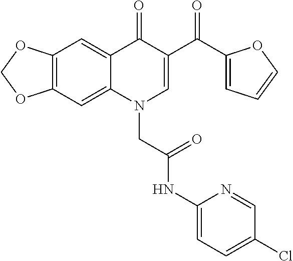 [SMILES: O=C(Cn1cc(C(=O)c2ccco2)c(=O)c2cc3c(cc21)OCO3)Nc1ccc(Cl)cn1]

The title compound was prepared in accordance with Example 1, using 2-amino-5-chloropyridine in step (e). The total yield starting from furoic acid methyl ester was 4%.

¹H NMR (400 MHz, DMSO-d₆) δ: 11.20 (s, 1H), 8.43 (d, J=2.8 Hz, 1H), 8.36 (s, 1H), 8.06-7.96 (m, 2H), 7.91 (dd, J=9.1, 2.9 Hz, 1H), 7.57 (s, 1H), 7.32 (d, J=3.7 Hz, 1H), 7.23 (s, 1H), 6.73-6.68 (m, 1H), 6.20 (s, 2H), 5.29 (s, 2H).

### Example 28

### N-(5-Chloro-2-pyridyl)-2-[6,7-methylenedioxy-3-(2-naphthoyl)-4-oxo-1-quinolinyl]-acetamide

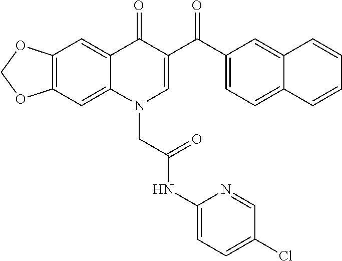 [SMILES: O=C(Cn1cc(C(=O)c2ccc3ccccc3c2)c(=O)c2cc3c(cc21)OCO3)Nc1ccc(Cl)cn1]

### (a) 2-naphthoylacetic acid ethyl ester

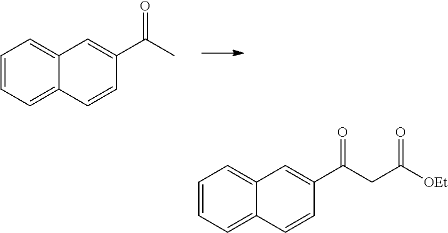 [SMILES: CC(=O)c1ccc2ccccc2c1 ; CCOC(=O)CC(=O)c1ccc2ccccc2c1]

Diethylcarbonate (195 g, 1.65 mol) was brought to reflux, the heating was removed and Na (5.75 g, 0.25 mol) was added in small portions. The mixture was heated to 100° C. for complete dissolution of the Na. 2-Acetylnaphthalene (4.26 g, 0.25 mol) was added in small portions and the temperature was slowly increased to 120° C. in order to distil off the formed EtOH. The mixture was cooled to rt; ice (80 g) and acetic acid (20 mL) were added. The layers were separated and the aqueous phase was extracted with Et₂O (2×100 mL). The combined organic phases were washed with water (2×100 mL), dried (Na₂SO₄), concentrated to give the sub-title compound which was used in the next step without further purification.

### (b) N-(5-Chloro-2-pyridyl)-2-[6,7-methylenedioxy-3-(2-naphthoyl)-4-oxo-1-quinolinyl]acetamide

The title compound was prepared from in accordance with Example 1, Steps (b) to (f) using 2-naphthoylacetic acid ethyl ester in Step (b) and 2-amino-5-chloropyridine in Step (e). The reaction time in Step (d) was 1 h. The total yield starting from 2-acetylnaphthalene was 10%.

¹H NMR (DMSO-d₆) δ: 10.34 (s, 1H), 8.37 (s, 1H), 7.98 (s, 1H), 7.57 (s, 1H), 7.51 (d, J=8.6 Hz, 2H), 7.32 (d, J=3.6 Hz, 1H), 7.21 (s, 1H), 6.90 (d, J=8.6 Hz, 2H), 6.74 ? 6.68 (m, 1H), 6.20 (s, 2H), 5.18 (s, 2H), 3.72 (s, 3H).

### Example 29-46

The title compounds were prepared from the appropriately substituted 2-aroyl- or 2-heteroaroyl-acetic acid ester (prepared as in Example 1, Step (a) or Example 28, Step (a)) and the appropriately substituted aniline or pyridylamine using the methods described herein, see Table 3.

TABLE 3

| | Chemical structure |
|---|---|
| | Name |
| Example | ¹H NMR (DMSO-d₆) δ |
| 29 | 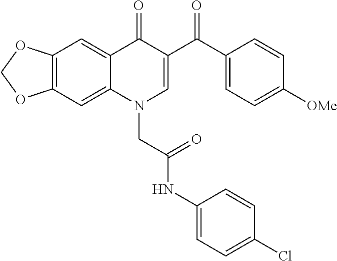 [SMILES: COc1ccc(C(=O)c2cn(CC(=O)Nc3ccc(Cl)cc3)c3cc4c(cc3c2=O)OCO4)cc1] 2-[3-p-Anisoyl-6,7-methylenedioxy-4-oxo-1-quinolinyl]-N- (4-chlorophenyl)acetamide 1H NMR (500 MHz, DMSO-d6) δ 10.69 (bs, 1H), 8.31 (s, 1H), 7.74 (d, J = 8.3 Hz, 2H), 7.63 (d, J = 8.5 Hz, 2H), 7.53 (s, 1H), 7.38 (d, J = 8.4 Hz, 2H), 7.24 (s, 1H), 7.00 (d, J = 8.4 Hz, 2H), 6.19 (s, 2H), 5.24 (s, 2H), 3.83 (s, 3H). |
| 30 | 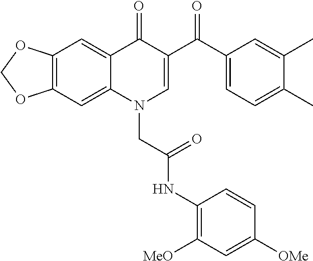 [SMILES: COc1ccc(NC(=O)Cn2cc(C(=O)c3ccc(C)c(C)c3)c(=O)c3cc4c(cc32)OCO4)c(OC)c1] N-(2,4-Dimethoxyphenyl)-2-[3-(3,4-dimethylbenzoyl)-6,7- methylenedioxy-4-oxo-1-quinolinyl]acetamide 1H NMR (500 MHz, DMSO-d6) δ 9.70 (s, 1H), 8.29 (s, 1H), 7.69 (d, J = 8.7 Hz, 1H), 7.53 (s, 2H), 7.46 (d, J = 7.8 Hz, 1H), 7.24- 7.19 (m, 2H), 6.65 (d, J = 2.7 Hz, 1H), 6.48 (dd, J = 8.9, 2.7 Hz, 1H), 6.21 (s, 2H), 5.27 (s, 2H), 3.86 (s, 3H), 3.74 (s, 3H), 2.30- 2.23 (m, 6H). |
| 31 | 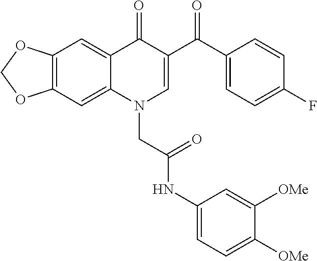 [SMILES: COc1ccc(NC(=O)Cn2cc(C(=O)c3ccc(F)cc3)c(=O)c3cc4c(cc32)OCO4)cc1OC] N-(3,4-Dimethoxyphenyl)-2-[3-(4-fluorobenzoyl)-6,7- methylenedioxy-4-oxo-1-quinolinyl]acetamide 1H NMR (500 MHz, DMSO-d6) δ 10.45 (s, 1H), 8.40 (s, 1H), 7.84-7.75 (m, 2H), 7.55-7.50 (m, 1H), 7.37-7.20 (m, 4H), 7.09 (d, J = 9.4 Hz, 1H), 6.91 (d, J = 8.3 Hz, 1H), 6.19 (s, 2H), 5.23 (s, 2H), 3.74-3.68 (m, 6H). |
| 32 | 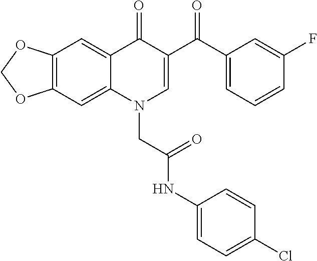 [SMILES: O=C(Cn1cc(C(=O)c2cccc(F)c2)c(=O)c2cc3c(cc21)OCO3)Nc1ccc(Cl)cc1] N-(4-Chlorophenyl)-2-[3-(3-fluorobenzoyl)-6,7- methylenedioxy-4-oxo-1-quinolinyl]acetamide 1H NMR (500 MHz, DMSO-d6) δ 8.45 (s, 1H), 7.63 (d, J = 8.4 Hz, 2H), 7.58-7.36 (m, 7H), 7.26 (s, 1H), 6.19 (s, 2H), 5.27 (s, 2H). |
| 33 | 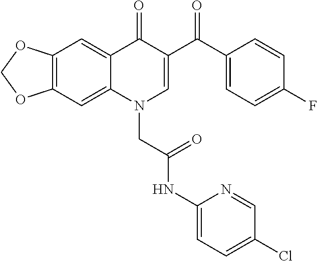 [SMILES: O=C(Cn1cc(C(=O)c2ccc(F)cc2)c(=O)c2cc3c(cc21)OCO3)Nc1ccc(Cl)cn1] N-(5-Chloro-2-pyridyl)-2-[3-(4-fluorobenzoyl)-6,7- methylenedioxy-4-oxo-1-quinolinyl]acetamide 1H NMR (500 MHz, DMSO-d6) δ 8.41-8.37 (m, 2H), 8.00 (d, J = 8.8 Hz, 1H), 7.89-7.77 (m, 3H), 7.52 (s, 1H), 7.33-7.24 (m, 3H), 6.19 (s, 2H), 5.29 (s, 2H). |
| 34 | 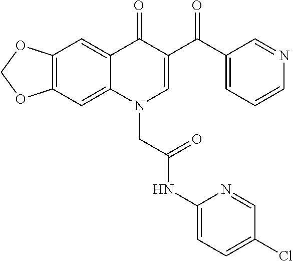 [SMILES: O=C(Cn1cc(C(=O)c2cccnc2)c(=O)c2cc3c(cc21)OCO3)Nc1ccc(Cl)cn1] N-(5-Chloro-2-pyridyl)-2-(6,7-methylenedioxy-3-nicotinoyl- 4-oxo-1-quinolinyl)acetamide 1H NMR (500 MHz, DMSO-d6) δ 11.26 (s, 1H), 8.81 (d, J = 2.3 Hz, 1H), 8.72 (dd, J = 5.0, 1.8 Hz, 1H), 8.52 (s, 1H), 8.44 (d, J = 2.7 Hz, 1H), 8.06-8.00 (m, 2H), 7.92 (dd, J = 8.9, 2.8 Hz, 1H), 7.54-7.47 (m, 2H), 7.29 (s, 1H), 6.20 (s, 2H), 5.39 (s, 2H). |
| 35 | 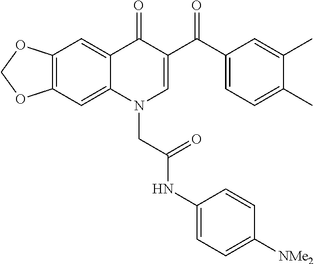 [SMILES: Cc1ccc(C(=O)c2cn(CC(=O)Nc3ccc(N(C)C)cc3)c3cc4c(cc3c2=O)OCO4)cc1C] N-(4-Dimethylaminophenyl)-3-[3,4-dimethylbenzoyl)-6,7- methylenedioxy-4-oxo-1-quinolinyl]acetamide 1H NMR (400 MHz, DMSO-d6) δ 10.19 (s, 1H), 8.30 (s, 1H), 7.55- 7.38 (m, 5H), 7.26-7.17 (m, 2H), 6.70 (d, J = 8.7 Hz, 2H), 6.19 (s, 2H), 5.17 (s, 2H), 2.84 (s, 6H), 2.31-2.23 (m, 6H). |
| 36 | 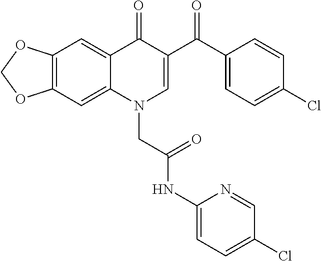 [SMILES: O=C(Cn1cc(C(=O)c2ccc(Cl)cc2)c(=O)c2cc3c(cc21)OCO3)Nc1ccc(Cl)cn1] 2-[3-(4-Chlorobenzoyl)-6,7-methylenedioxy-4-oxo-1-quinolinyl]- N-(5-chloro-2-pyridyl)acetamide 1H NMR (400 MHz, DMSO-d6) δ 11.21 (s, 1H), 8.46-8.40 (m, 2H), 8.03 (d, J = 9.1 Hz, 1H), 7.92 (dd, J = 8.9, 2.7 Hz, 1H), 7.72 (d, J = 8.3 Hz, 2H), 7.57-7.47 (m, 3H), 7.25 (s, 1H), 6.19 (s, 2H), 5.33 (s, 2H). |
| 37 | 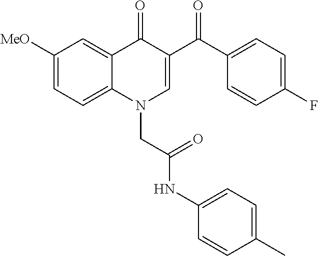 [SMILES: COc1ccc2c(c1)c(=O)c(C(=O)c1ccc(F)cc1)cn2CC(=O)Nc1ccc(C)cc1] 2-[3-(4-fluorobenzoyl)-6-methoxy-4-oxo-1-quinolinyl]-N-p- tolylacetamide 2.25 (s, 3H); 3.86 (s, 3H); 5.29 (s, 2H); 7.11-7.15 (m, 2H); 7.25- 7.33 (m, 2H); 7.43 (dd, J = 3.0, 9.2 Hz, 1H); 7.46-7.50 (m, 2H); 7.59 (d, J = 9.2 Hz, 1H); 7.65 (d, J = 3.0 Hz, 1H); 7.78-7.84 (m, 2H); 8.49 (s. 1H); 10.47 (s, 1H) |
| 38 | 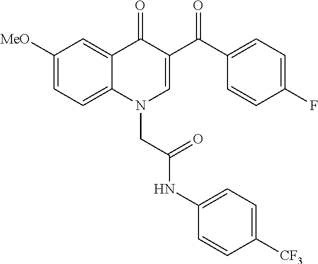 [SMILES: COc1ccc2c(c1)c(=O)c(C(=O)c1ccc(F)cc1)cn2CC(=O)Nc1ccc(C(F)(F)F)cc1] 2-[3-(4-Fluorobenzoyl)-6-methoxy-4-oxo-1-quinolinyl]-N- (4-trifluoromethylphenyl)acetamide 3.85 (s, 3H); 5.36 (s, 2H); 7.27-7.33 (m, 2H); 7.42 (dd, J = 3.0, 9.2 Hz, 1H); 7.61 (d, J = 9.2 Hz, 1H); 7.65 (d, J = 3.0 Hz, 1H); 7.69-7.73 (m, 2H); 7.78-7.84 (m, 4H); 8.51 (s, 1H); 10.92 (s, 1H) |
| 39 | 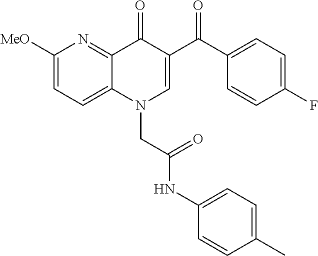 [SMILES: COc1ccc2c(n1)c(=O)c(C(=O)c1ccc(F)cc1)cn2CC(=O)Nc1ccc(C)cc1] 2-[3-(4-Fluorobenzoyl)-6-methoxy-4-oxo-1,5-naphthyridin-1- yl]-N-p-tolylacetamide 10.43 (s, 1H), 8.47 (s, 1H), 8.11-8.05 (m, 1H), 7.88-7.76 (m, 2H), 7.47 (d, J = 8.1 Hz, 2H), 7.35-7.25 (m, 3H), 7.13 (d, J = 8.1 Hz, 2H), 5.30 (s, 2H), 3.96 (s, 3H), 2.25 (s, 3H). |
| 40 | 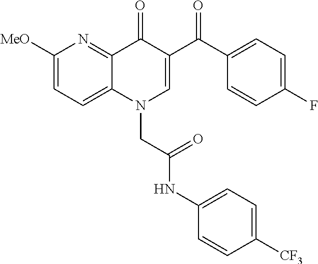 [SMILES: COc1ccc2c(n1)c(=O)c(C(=O)c1ccc(F)cc1)cn2CC(=O)Nc1ccc(C(F)(F)F)cc1] 2-[3-(4-Fluorobenzoyl)-6-methoxy-4-oxo-1,5-naphthyridin-1- yl]-N-(4-trifluoromethylphenyl)acetamide 3.96 (s, 3H); 5.37 (s, 2H); 7.25-7.35 (m, 3H); 7.68-7.73 (m, 2H); 7.71-7.86 (m, 4H); 8.10-8.15 (m, 1H); 8.49 (s, 1H); 10.88 (s, 1H) |
| 41 | 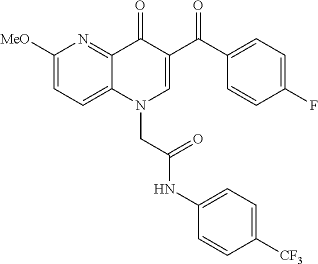 [SMILES: COc1ccc2c(n1)c(=O)c(C(=O)c1ccc(F)cc1)cn2CC(=O)Nc1ccc(C(F)(F)F)cc1] N-(4-Cyanophenyl)-2-[3-(4-fluorobenzoyl)-6-methoxy-4-oxo-1,5- naphthyridin-1-yl]-acetamide 3.96 (s, 3H); 5.36 (s, 2H); 7.22-7.37 (m, 3H); 7.70-7.88 (m, 6H); 8.09-8.16 (m, 1H); 8.47 (s, 1H); 10.95 (s, 1H) |
| 42 | 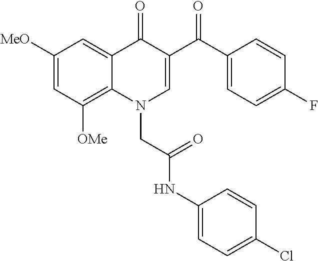 [SMILES: COc1cc(OC)c2c(c1)c(=O)c(C(=O)c1ccc(F)cc1)cn2CC(=O)Nc1ccc(Cl)cc1] N-(4-Chlorophenyl)-2-[6,8-dimethoxy-3-(4-fluorobenzoyl)-4- oxo-1-quinolinyl]acetamide 3.78 (s, 3H); 3.82 (s, 3H); 5.15 (s, 2H); 6.43 (d, J = 2.1 Hz, 1H); 6.53 (d, J = 2.1 Hz, 1H); 7.29 (distorted t, J = 8.9 Hz, 2H); 7.40 (AA′BB′ mult., J = 8.9 Hz, 2H); 7.61 (AA′BB′ mult., J = 8.9 Hz, 2H); 7.77 (distorted dd, J = 5.7, 8.8 Hz, 2H); 8.25 (s, 1H); 10.68 (s, 1H) |
| 43 | 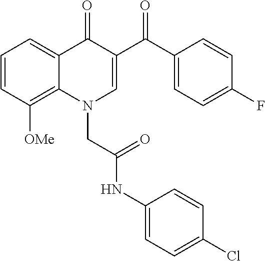 [SMILES: COc1cccc2c(=O)c(C(=O)c3ccc(F)cc3)cn(CC(=O)Nc3ccc(Cl)cc3)c12] N-(4-Chlorophenyl)-2-[3-(4-fluorobenzoyl-8-methoxy-4- oxo-1-quinolinyl]acetamide 3.79 (s, 3H); 5.40 (s, 2H); 7.30 (distorted t, J = 8.9 Hz, 2H); 7.37 (dd, J = 2.0, 8.1 Hz, 1H); 7.39 (AA′BB′ mult., J = 8.9 Hz, 2H); 7.40 (t, J = 7.7 Hz, 1H); 7.63 (AA′BB′ mult., J = 8.9 Hz, 2H); 7.81 (distorted dd, J = 5.6, 8.9 Hz, 2H); 7.84 (dd, J = 2.0, 7.4 Hz, 1H); 8.36 (s, 1H); 10.45 (s, 1H) |
| 44 | 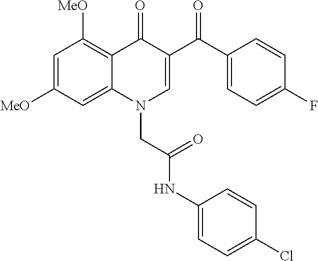 [SMILES: COc1cc(OC)c2c(=O)c(C(=O)c3ccc(F)cc3)cn(CC(=O)Nc3ccc(Cl)cc3)c2c1] N-(4-Chlorophenyl)-2-[4,6-dimethoxy-3-(4-fluorobenzoyl)-4- oxo-1-quinolinyl]acetamide 3.78 (s, 3H); 3.82 (s, 3H); 5.15 (s, 2H); 6.43 (d, J = 2.1 Hz, 1H); 6.53 (d, J = 2.1 Hz, 1H); 7.29 (distorted t, J = 8.9 Hz, 2H); 7.40 (AA′BB′ mult., J = 8.9 Hz, 2H); 7.61 (AA′BB′ mult., J = 8.9 Hz, 2H); 7.77 (distorted dd, J = 5.7, 8.8 Hz, 2H); 8.25 (s, 1H); 10.68 (s, 1H) |

### Example 45

### N-p-Anisyl-2-[3-(4-chlorobenzenesulfonyl)-6,7-methylenedioxy-4-oxo-1-quinolinyl]acetamide

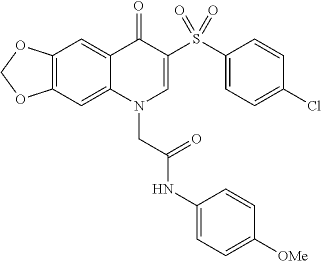 [SMILES: COc1ccc(NC(=O)Cn2cc(S(=O)(=O)c3ccc(Cl)cc3)c(=O)c3cc4c(cc32)OCO4)cc1]

### (a) 2-(4-Chlorophenylsulfonyl)acetic acid methyl ester

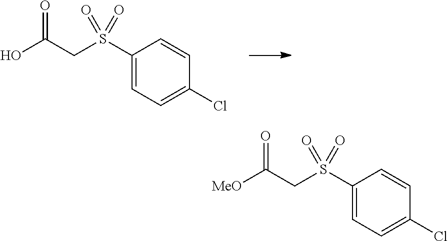 [SMILES: O=C(O)CS(=O)(=O)c1ccc(Cl)cc1 ; COC(=O)CS(=O)(=O)c1ccc(Cl)cc1]

Diisopropylethylamine (10.9 g, 84 mmol) was added to a mixture of 2-(4-chloro-phenylsulfonyl)acetic acid (15.0 g, 64 mmol) and anhydrous MeCN (100 ml). Methyl iodide (10.9 g, 77 mmol) was added dropwise at rt and the mixture was stirred at 60° C. for 6 h. The mixture was concentrated, poured into water and extracted with CH₂Cl₂ (150 mL). The extract was washed twice with water (200 ml), dried (Na₂SO₄), and concentrated to give the sub-title compound (14.3 g, 90%).

### (b) N-p-Anisyl-2-[3-(4-chlorobenzenesulfonyl)-6,7-methylenedioxy-4-oxo-1-quinolinyl]acetamide

The title compound was prepared from in accordance with Example 1, using 2-(4-chlorophenylsulfonyl)acetic acid methyl ester in step (b). The reaction time in step (d) was 0.5 h. The total yield starting from 2-(4-chlorophenylsulfonyl)acetic acid was 22%.

¹H NMR (400 MHz, DMSO-d₆) δ: 10.38 (s, 1H), 8.85 (s, 1H), 8.04 (d, J=8.4 Hz, 2H), 7.68 (d, J=8.3 Hz, 2H), 7.52 (d, J=8.6 Hz, 2H), 7.46 (s, 1H), 7.24 (s, 1H), 6.91 (d, J=8.6 Hz, 2H), 6.20 (s, 2H), 5.32 (s, 2H), 3.72 (s, 3H).

In addition to the compounds synthesized as described above, certain compounds in Table 4 were commercially available and purchased from ChemDiv or Enamine.

TABLE 4

| | Chemical structure |
|---|---|
| Example | Name |
| 46 | 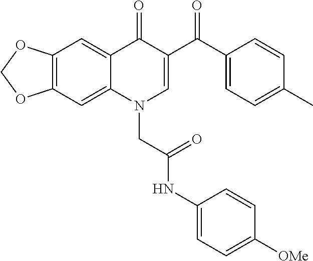 [SMILES: COc1ccc(NC(=O)Cn2cc(C(=O)c3ccc(C)cc3)c(=O)c3cc4c(cc32)OCO4)cc1] N-p-Anisyl-2-(6,7-methylenedioxy-4-oxo-3-p-toluoyl- 1-quinolinyl)acetamide |
| 47 | 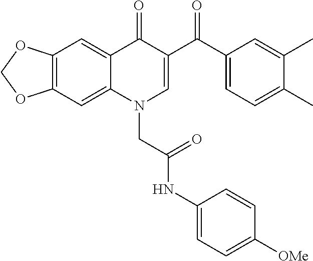 [SMILES: COc1ccc(NC(=O)Cn2cc(C(=O)c3ccc(C)c(C)c3)c(=O)c3cc4c(cc32)OCO4)cc1] N-p-Anisyl-2-[3-(3,4-dimethylbenzoyl)-6,7-methyl- enedioxy-4-oxo-1-quinolinyl]acetamide |
| 48 | 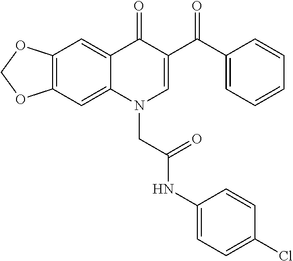 [SMILES: O=C(Cn1cc(C(=O)c2ccccc2)c(=O)c2cc3c(cc21)OCO3)Nc1ccc(Cl)cc1] 2-(3-Benzoyl-6,7-methylenedioxy-4-oxo-1- quinolinyl)-N-(4-chlorophenyl)acetamide |
| 49 | 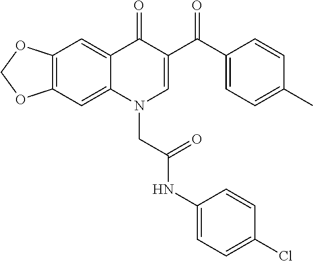 [SMILES: Cc1ccc(C(=O)c2cn(CC(=O)Nc3ccc(Cl)cc3)c3cc4c(cc3c2=O)OCO4)cc1] N-(4-Chlorophenyl)-2-(3-p-toluoyl-6,7-methylene- dioxy-4-oxo-1-quinolinyl)acetamide |
| 50 | 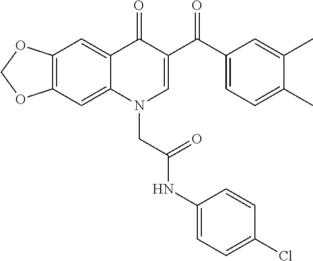 [SMILES: Cc1ccc(C(=O)c2cn(CC(=O)Nc3ccc(Cl)cc3)c3cc4c(cc3c2=O)OCO4)cc1C] N-(4-Chlorophenyl)-2-[3-(3,4-dimethylbenzoyl)-6,7- methylenedioxy-4-oxo-1-quinolinyl]acetamide |
| 51 | 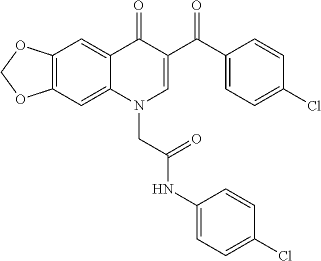 [SMILES: O=C(Cn1cc(C(=O)c2ccc(Cl)cc2)c(=O)c2cc3c(cc21)OCO3)Nc1ccc(Cl)cc1] 2-[3-(4-Chlorobenzoyl)-6,7-methylenedioxy-4-oxo-1- quinolinyl]-N-(4-chlorophenyl)acetamide |
| 52 | 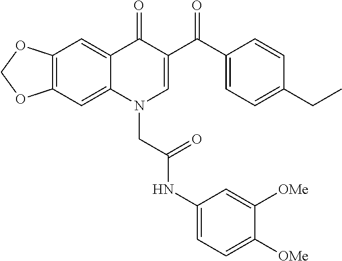 [SMILES: CCc1ccc(C(=O)c2cn(CC(=O)Nc3ccc(OC)c(OC)c3)c3cc4c(cc3c2=O)OCO4)cc1] N-(3,4-Dimethoxyphenyl)-2-[3-(4-ethylbenzoyl)-6,7- methylenedioxy-4-oxo-1-quinolinyl]acetamide |
| 53 | 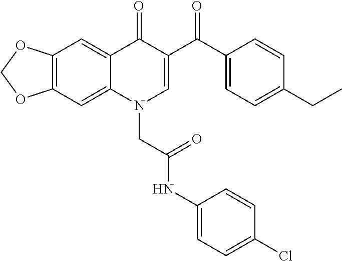 [SMILES: CCc1ccc(C(=O)c2cn(CC(=O)Nc3ccc(Cl)cc3)c3cc4c(cc3c2=O)OCO4)cc1] N-(4-Chlorophenyl)-2-[3-(4-ethylbenzoyl)-6,7- methylenedioxy-4-oxo-1-quinolinyl]acetamide |
| 54 | 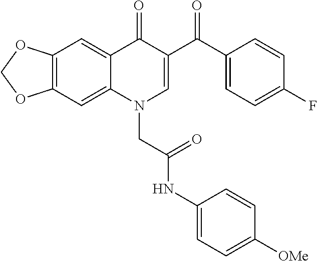 [SMILES: COc1ccc(NC(=O)Cn2cc(C(=O)c3ccc(F)cc3)c(=O)c3cc4c(cc32)OCO4)cc1] N-p-Anisyl-2-[(4-fluorobenzoyl)-6,7-methylenedioxy- 4-oxo-1-quinolinyl]acetamide |
| 55 | 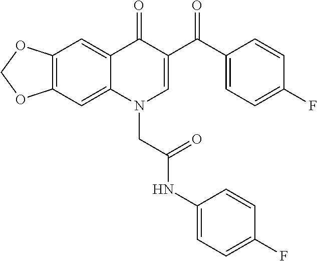 [SMILES: O=C(Cn1cc(C(=O)c2ccc(F)cc2)c(=O)c2cc3c(cc21)OCO3)Nc1ccc(F)cc1] 2-[3-(4-Fluorobenzoyl)-6,7-methylenedioxy-4-oxo-1- quinolinyl]-N-(4-fluorophenyl)acetamide |
| 56 | 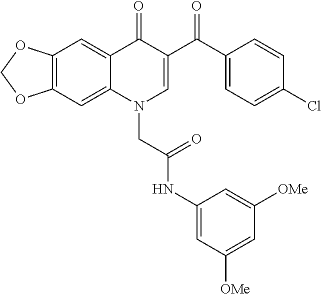 [SMILES: COc1cc(NC(=O)Cn2cc(C(=O)c3ccc(Cl)cc3)c(=O)c3cc4c(cc32)OCO4)cc(OC)c1] 2-[3-(4-Chlorobenzoyl)-6,7-methylenedioxy-4-oxo-1- quinolinyl]-N-(3,5-dimethoxyphenyl)acetamide |
| 57 | 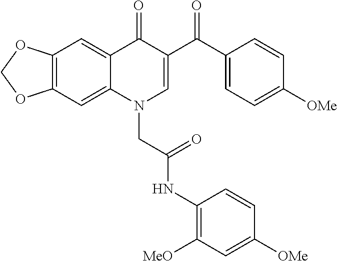 [SMILES: COc1ccc(C(=O)c2cn(CC(=O)Nc3ccc(OC)cc3OC)c3cc4c(cc3c2=O)OCO4)cc1] 2-(3-p-Anisoyl-6,7-methylenedioxy-4-oxo-1- quinolinyl)-N-(3,5-dimethoxyphenyl)acetamide |
| 58 | 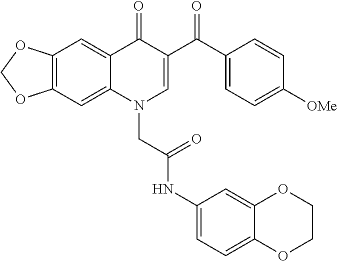 [SMILES: COc1ccc(C(=O)c2cn(CC(=O)Nc3ccc4c(c3)OCCO4)c3cc4c(cc3c2=O)OCO4)cc1] 2-(3-p-Anisoyl-67-methylenedioxy-4-oxo-1- quinolinyl)-N-(3,4-ethylenedioxyphenyl)acetamide |
| 59 | 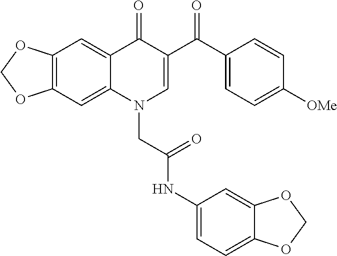 [SMILES: COc1ccc(C(=O)c2cn(CC(=O)Nc3ccc4c(c3)OCO4)c3cc4c(cc3c2=O)OCO4)cc1] 2-(3-p-Anisoyl-6,7-methylenedioxy-4-oxo-1- quinolinyl)-N-(3,4-methylenedioxyphenyl)acetamide |
| 60 | 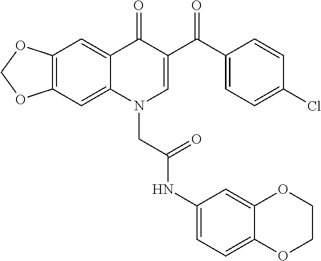 [SMILES: O=C(Cn1cc(C(=O)c2ccc(Cl)cc2)c(=O)c2cc3c(cc21)OCO3)Nc1ccc2c(c1)OCCO2] 2-[3-(4-Chlorobenzoyl)-6,7-methylenedioxy-4-oxo-1- quinolinyl]-N-(3,4-ethylenedioxyphenyl)acetamide |
| 61 | 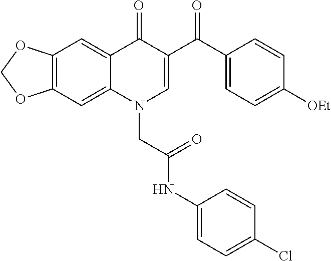 [SMILES: CCOc1ccc(C(=O)c2cn(CC(=O)Nc3ccc(Cl)cc3)c3cc4c(cc3c2=O)OCO4)cc1] N-(4-Chlorophenyl)-2-[3-(4-ethoxybenzoyl)-6,7- methylenedioxy-4-oxo-1-quinolinyl]acetamide |
| 62 | 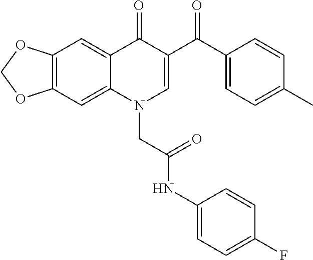 [SMILES: Cc1ccc(C(=O)c2cn(CC(=O)Nc3ccc(F)cc3)c3cc4c(cc3c2=O)OCO4)cc1] N-(4-Fluorophenyl)-2-(6,7-methylenedioxy-4-oxo-3- p-toluoyl-1-quinolinyl)acetamide |
| 63 | 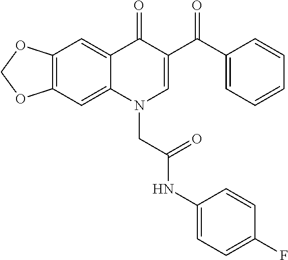 [SMILES: O=C(Cn1cc(C(=O)c2ccccc2)c(=O)c2cc3c(cc21)OCO3)Nc1ccc(F)cc1] 2-(3-Benzoyl-6,7-methylenedioxy-4-oxo-1- quinolinyl)-N-(4-fluorophenyl)acetamide |
| 64 | 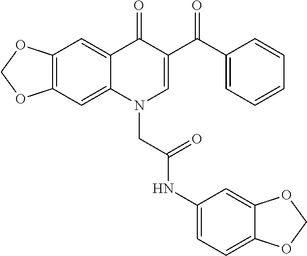 [SMILES: O=C(Cn1cc(C(=O)c2ccccc2)c(=O)c2cc3c(cc21)OCO3)Nc1ccc2c(c1)OCO2] 2-(3-Benzoyl-6,7-methylenedioxy-4-oxo-1- quinolinyl)-N-(3,4-methylenedioxyphenyl)acetamide |
| 65 | 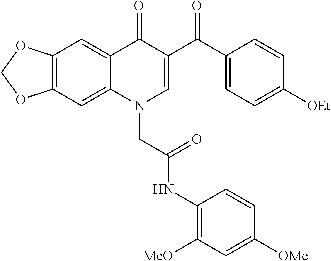 [SMILES: CCOc1ccc(C(=O)c2cn(CC(=O)Nc3ccc(OC)cc3OC)c3cc4c(cc3c2=O)OCO4)cc1] N-(2,4-Dimethoxyphenyl)-2-[3-(4-ethoxybenzoyl)- 6,7-methylenedioxy-4-oxo-1-quinolinyl]acetamide |
| 66 | 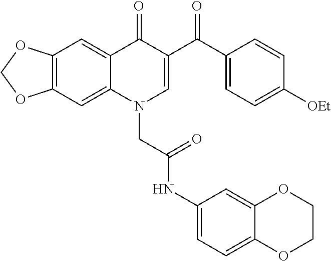 [SMILES: CCOc1ccc(C(=O)c2cn(CC(=O)Nc3ccc4c(c3)OCCO4)c3cc4c(cc3c2=O)OCO4)cc1] 2-[3-(4-Ethoxybenzoyl)-6,7-methylenedioxy-4-oxo-1- quinolinyl]-N-(3,4-ethylenedioxyphenyl)acetamide |
| 67 | 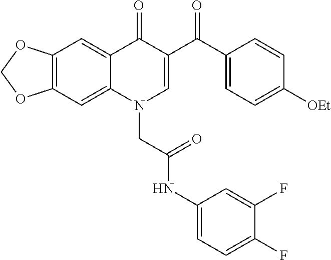 [SMILES: CCOc1ccc(C(=O)c2cn(CC(=O)Nc3ccc(F)c(F)c3)c3cc4c(cc3c2=O)OCO4)cc1] N-(3,4-Difluorophenyl)-2-[3-(4-ethoxybenzoyl)-6,7- methylenedioxy-4-oxo-1-quinolinyl]acetamide |
| 68 | 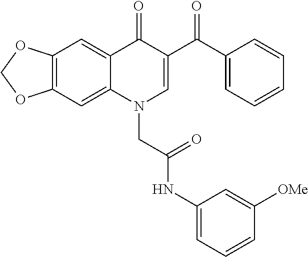 [SMILES: COc1cccc(NC(=O)Cn2cc(C(=O)c3ccccc3)c(=O)c3cc4c(cc32)OCO4)c1] N-(m-Anisyl)-2-(3-benzoyl-6,7-methylenedioxy-4- oxo-1-quinolinyl)acetamide |
| 69 | 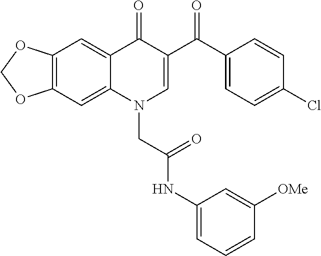 [SMILES: COc1cccc(NC(=O)Cn2cc(C(=O)c3ccc(Cl)cc3)c(=O)c3cc4c(cc32)OCO4)c1] N-(m-Anisyl)-2-[3-(4-chlorobenzoyl)-6,7- methylenedioxy-4-oxo-1-quinolinyl]acetamide |
| 70 | 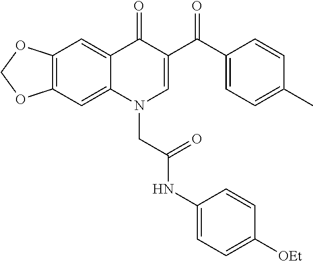 [SMILES: CCOc1ccc(NC(=O)Cn2cc(C(=O)c3ccc(C)cc3)c(=O)c3cc4c(cc32)OCO4)cc1] N-(4-Ethoxyphenyl)-2-(6,7-methylenedioxy-4-oxo-3- p-toluoyl-1-quinolinyl)acetamide |
| 71 | 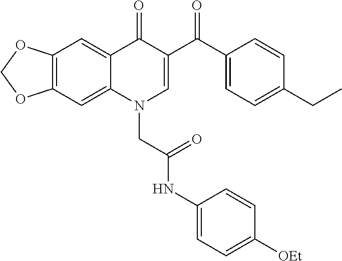 [SMILES: CCOc1ccc(NC(=O)Cn2cc(C(=O)c3ccc(CC)cc3)c(=O)c3cc4c(cc32)OCO4)cc1] N-(4-Ethoxyphenyl)-2-[3-(4-ethylbenzoyl)-6,7- methylenedioxy-4-oxo-1-quinolinyl]acetamide |
| 72 | 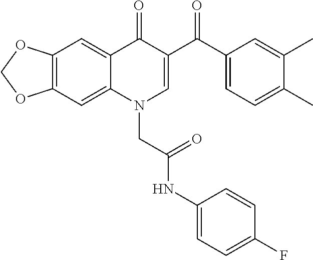 [SMILES: Cc1ccc(C(=O)c2cn(CC(=O)Nc3ccc(F)cc3)c3cc4c(cc3c2=O)OCO4)cc1C] 2-[3-(3,4-Dimethylbenzoyl)-6,7-methylenedioxy-4- oxo-1-quinolinyl]-N-(4-fluorophenyl)acetamide |
| 73 | 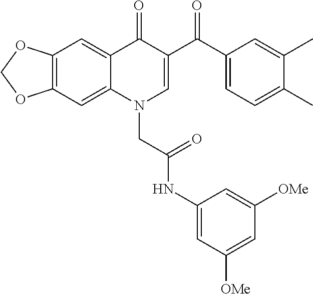 [SMILES: COc1cc(NC(=O)Cn2cc(C(=O)c3ccc(C)c(C)c3)c(=O)c3cc4c(cc32)OCO4)cc(OC)c1] N-(3,5-Dimethoxyphenyl)-2-[3-(3,4- dimethylbenzoyl)-6,7-methylenedioxy-4-oxo-1- quinolinyl]acetamide |
| 74 | 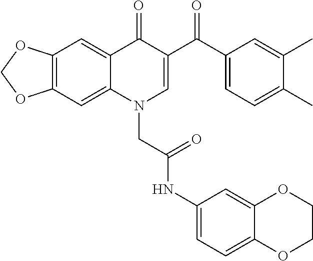 [SMILES: Cc1ccc(C(=O)c2cn(CC(=O)Nc3ccc4c(c3)OCCO4)c3cc4c(cc3c2=O)OCO4)cc1C] 2-[3-(3,4-Dimethylbenzoyl)-6,7-methylenedioxy-4- oxo-1-quinolinyl]-N-(3,4-ethylenedioxyphenyl)- acetamide |
| 75 | 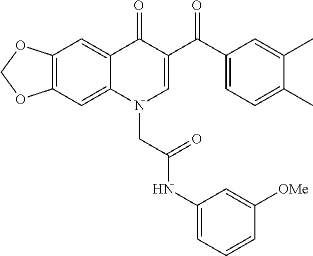 [SMILES: COc1cccc(NC(=O)Cn2cc(C(=O)c3ccc(C)c(C)c3)c(=O)c3cc4c(cc32)OCO4)c1] N-m-Anisyl-2-[3-(3,4-dimethylbenzoyl)-6,7-methyl- enedioxy-4-oxo-1-quinolinyl]acetamide |
| 76 | 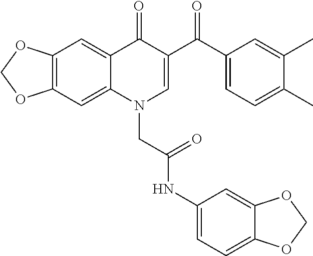 [SMILES: Cc1ccc(C(=O)c2cn(CC(=O)Nc3ccc4c(c3)OCO4)c3cc4c(cc3c2=O)OCO4)cc1C] 2-[3-(3,4-Dimethylbenzoyl)-6,7-methylenedioxy-4- oxo-1-quinolinyl]-N-(3,4-methylenedioxyphenyl)- acetamide |
| 77 | 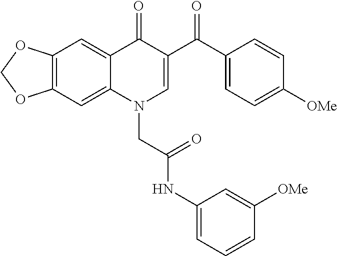 [SMILES: COc1ccc(C(=O)c2cn(CC(=O)Nc3cccc(OC)c3)c3cc4c(cc3c2=O)OCO4)cc1] 2-(3-p-Anisoyl-6,7-methylenedioxy-4-oxo-1-quinolin- yl)-N-m-anisylacetamide |
| 78 | 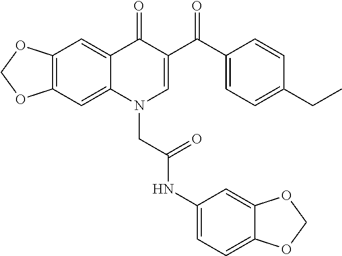 [SMILES: CCc1ccc(C(=O)c2cn(CC(=O)Nc3ccc4c(c3)OCO4)c3cc4c(cc3c2=O)OCO4)cc1] 2-[3-(4-Ethylbenzoyl)-6,7-methylenedioxy-4-oxo-1- quinolinyl]-N-(3,4-methylenedioxyphenyl)acetamide |
| 79 | 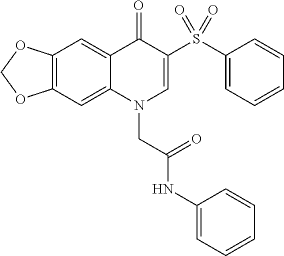 [SMILES: O=C(Cn1cc(S(=O)(=O)c2ccccc2)c(=O)c2cc3c(cc21)OCO3)Nc1ccccc1] 2-(3-Benzenesulfonyl-6,7-methylenedioxy-4-oxo-1- quinolinyl)-N-phenylacetamide |
| 80 | 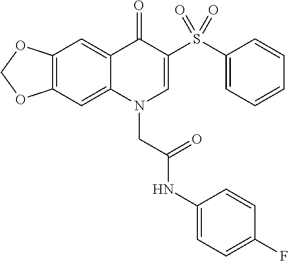 [SMILES: O=C(Cn1cc(S(=O)(=O)c2ccccc2)c(=O)c2cc3c(cc21)OCO3)Nc1ccc(F)cc1] 2-(3-Benzenesulfonyl-6,7-methylenedioxy-4-oxo-1- quinolinyl)-N-(4-fluorophenyl)acetamide |
| 81 | 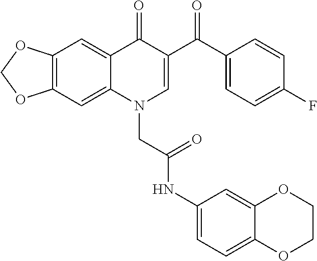 [SMILES: O=C(Cn1cc(C(=O)c2ccc(F)cc2)c(=O)c2cc3c(cc21)OCO3)Nc1ccc2c(c1)OCCO2] N-(3,4-Ethylenedioxyphenyl)-2-[3-(4-fluorobenzoyl)- 6,7-methylenedioxy-4-oxo-1-quinolinyl]acetamide |
| 82 | 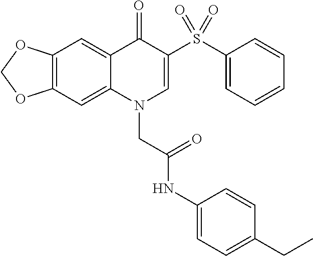 [SMILES: CCc1ccc(NC(=O)Cn2cc(S(=O)(=O)c3ccccc3)c(=O)c3cc4c(cc32)OCO4)cc1] 2-(3-Benzenesulfonyl-6,7-methylenedioxy-4-oxo-1- quinolinyl)-N-(4-ethylphenyl)acetamide |
| 83 | 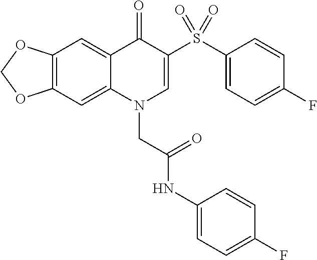 [SMILES: O=C(Cn1cc(S(=O)(=O)c2ccc(F)cc2)c(=O)c2cc3c(cc21)OCO3)Nc1ccc(F)cc1] 2-[3-(4-Fluorobenzenesulfonyl)-6,7-methylenedioxy- 4-oxo-1-quinolinyl]-N-(4-fluorophenyl)acetamide |
| 84 | 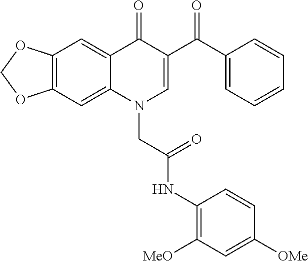 [SMILES: COc1ccc(NC(=O)Cn2cc(C(=O)c3ccccc3)c(=O)c3cc4c(cc32)OCO4)c(OC)c1] 2-(3-Benzoyl-6,7-methylenedioxy-4-oxo-1- quinolinyl)-N-(2,4-dimethoxyphenyl)acetamide |
| 85 | 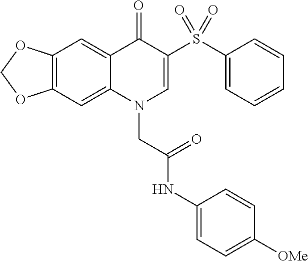 [SMILES: COc1ccc(NC(=O)Cn2cc(S(=O)(=O)c3ccccc3)c(=O)c3cc4c(cc32)OCO4)cc1] N-p-Anisyl-2-(3-benzenesulfonyl-6,7- methylenedioxy-4-oxo-1-quinolinyl)acetamide |
| 86 | 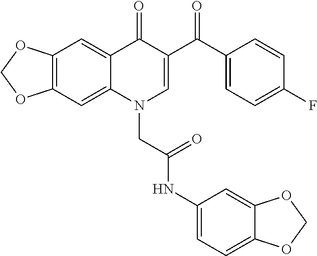 [SMILES: O=C(Cn1cc(C(=O)c2ccc(F)cc2)c(=O)c2cc3c(cc21)OCO3)Nc1ccc2c(c1)OCO2] 2-[3-(4-Fluorobenzoyl)-6,7-methylenedioxy-4-oxo-1- quinolinyl]-N-(3,4-methylenedioxyphenyl)acetamide |
| 87 | 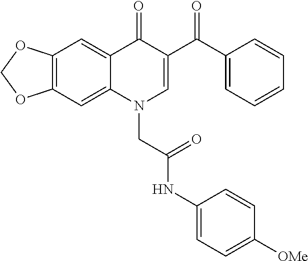 [SMILES: COc1ccc(NC(=O)Cn2cc(C(=O)c3ccccc3)c(=O)c3cc4c(cc32)OCO4)cc1] N-p-Anisyl-2-(3-benzoyl-6,7-methylenedioxy-4-oxo- 1 -quinolinyl)acetamide |
| 88 | 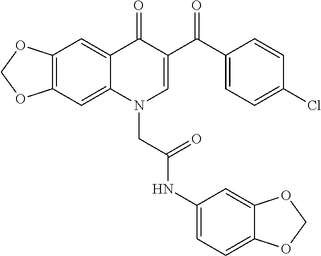 [SMILES: O=C(Cn1cc(C(=O)c2ccc(Cl)cc2)c(=O)c2cc3c(cc21)OCO3)Nc1ccc2c(c1)OCO2] 2-[3-(4-Chlorobenzoyl)-6,7-methylenedioxy-4-oxo-1- quinolinyl]-N-(3,4-methylenedioxyphenyl)acetamide |
| 89 | 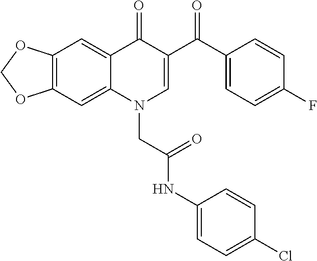 [SMILES: O=C(Cn1cc(C(=O)c2ccc(F)cc2)c(=O)c2cc3c(cc21)OCO3)Nc1ccc(Cl)cc1] N-(4-Chlorophenyl)-2-[3-(4-fluorobenzoyl)-6,7- methylenedioxy-4-oxo-1-quinolinyl]acetamide |
| 90 | 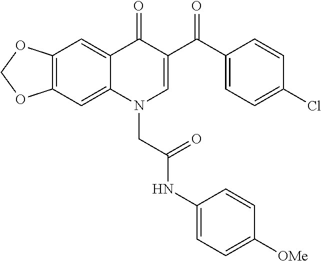 [SMILES: COc1ccc(NC(=O)Cn2cc(C(=O)c3ccc(Cl)cc3)c(=O)c3cc4c(cc32)OCO4)cc1] N-p-Anisyl-2-[3-(4-chlorobenzoyl)-6,7-methylene- dioxy-4-oxo-1-quinolinyl]acetamide |
| 91 | 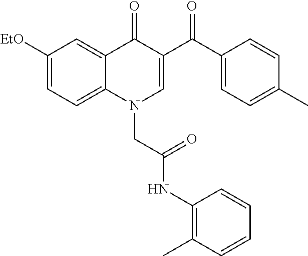 [SMILES: CCOc1ccc2c(c1)c(=O)c(C(=O)c1ccc(C)cc1)cn2CC(=O)Nc1ccccc1C] 2-(6-Ethoxy-4-oxo-3-p-toluoyl-1-quinolinyl)-N-o- tolylacetamide |
| 92 | 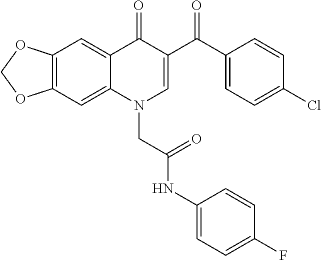 [SMILES: O=C(Cn1cc(C(=O)c2ccc(Cl)cc2)c(=O)c2cc3c(cc21)OCO3)Nc1ccc(F)cc1] 2-[3-(4-Chlorobenzoyl)-6,7-methylenedioxy-4-oxo-1- quinolinyl]-N-(4-fluorophenyl)acetamide |
| 93 | 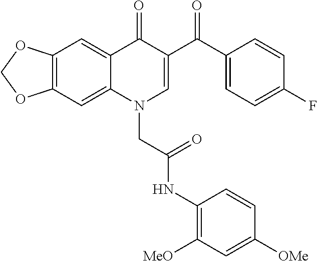 [SMILES: COc1ccc(NC(=O)Cn2cc(C(=O)c3ccc(F)cc3)c(=O)c3cc4c(cc32)OCO4)c(OC)c1] N-(2,4-Dimethoxyphenyl)-2-[3-(4-fluorobenzoyl)-6,7- methylenedioxy-4-oxo-1-quinolinyl]acetamide |
| 94 | 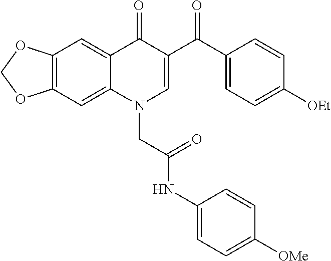 [SMILES: CCOc1ccc(C(=O)c2cn(CC(=O)Nc3ccc(OC)cc3)c3cc4c(cc3c2=O)OCO4)cc1] N-p-Anisyl-2-[3-(4-ethoxybenzoyl)-6,7-methylene- dioxy-4-oxo-1-quinolinyl]acetamide |
| 95 | 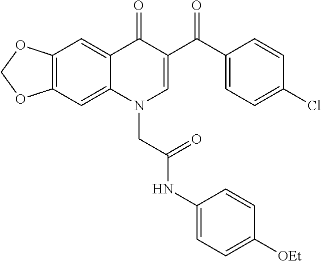 [SMILES: CCOc1ccc(NC(=O)Cn2cc(C(=O)c3ccc(Cl)cc3)c(=O)c3cc4c(cc32)OCO4)cc1] N-(4-Chlorophenyl)-2-[3-(4-ethoxybenzoyl)-6,7- methylenedioxy-4-oxo-1-quinolinyl]acetamide |
| 96 | 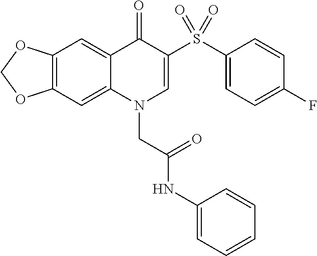 [SMILES: O=C(Cn1cc(S(=O)(=O)c2ccc(F)cc2)c(=O)c2cc3c(cc21)OCO3)Nc1ccccc1] 2-[3-(4-Fluorobenzenesulfonyl)-6,7-methylenedioxy- 4-oxo-1-quinolinyl]-N-phenylacetamide |
| 97 | 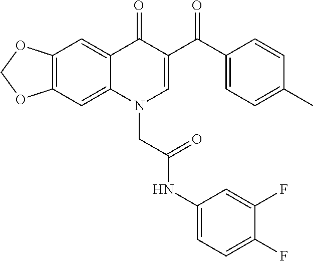 [SMILES: Cc1ccc(C(=O)c2cn(CC(=O)Nc3ccc(F)c(F)c3)c3cc4c(cc3c2=O)OCO4)cc1] N-(3,4-Difluorophenyl)-2-(6,7-methylenedioxy-4-oxo- 3-p-toluoyl-1-quinolinyl)acetamide |
| 98 | 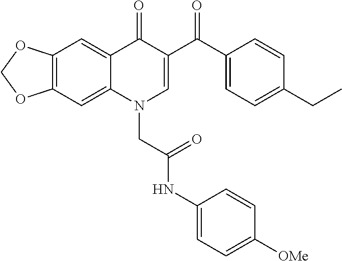 [SMILES: CCc1ccc(C(=O)c2cn(CC(=O)Nc3ccc(OC)cc3)c3cc4c(cc3c2=O)OCO4)cc1] N-p-Anisyl-2-[3-(4-ethylbenzoyl)-6,7-methylene- dioxy-4-oxo-1-quinolinyl]acetamide |
| 99 | 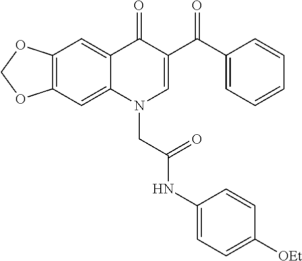 [SMILES: CCOc1ccc(NC(=O)Cn2cc(C(=O)c3ccccc3)c(=O)c3cc4c(cc32)OCO4)cc1] 2-(3-Benzoyl-6,7-methylenedioxy-4-oxo-1- quinolinyl)-N-(4-ethoxyphenyl)acetamide |
| 100 | 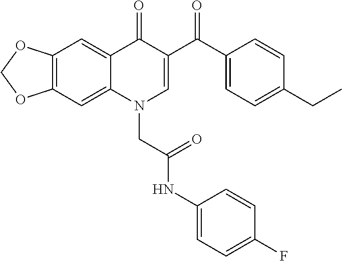 [SMILES: CCc1ccc(C(=O)c2cn(CC(=O)Nc3ccc(F)cc3)c3cc4c(cc3c2=O)OCO4)cc1] 2-[3-(4-Ethylbenzoyl)-6,7-methylenedioxy-4-oxo-1- quinolinyl]-N-(4-fluorophenyl)acetamide |
| 101 | 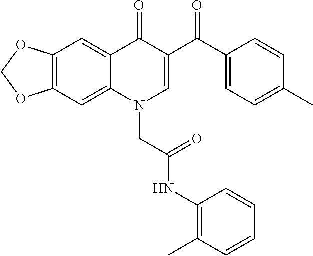 [SMILES: Cc1ccc(C(=O)c2cn(CC(=O)Nc3ccccc3C)c3cc4c(cc3c2=O)OCO4)cc1] 2-(6,7-Methylenedioxy-4-oxo-3-p-toluoyl-1- quinolinyl)-N-o-tolylacetamide |
| 102 | 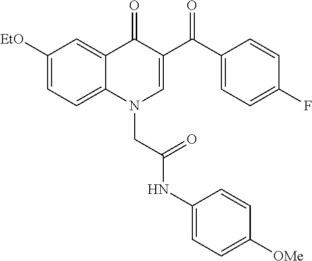 [SMILES: CCOc1ccc2c(c1)c(=O)c(C(=O)c1ccc(F)cc1)cn2CC(=O)Nc1ccc(OC)cc1] N-p-Anisyl-2-[6-ethoxy-3-(4-fluorobenzoyl)-4-oxo-1- quinolinyl]acetamide |
| 103 | 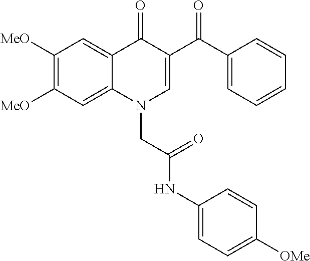 [SMILES: COc1ccc(NC(=O)Cn2cc(C(=O)c3ccccc3)c(=O)c3cc(OC)c(OC)cc32)cc1] N-p-Anisyl-2-(3-benzoyl-6,7-dimethoxy-4-oxo-1- quinolinyl)acetamide |
| 104 | 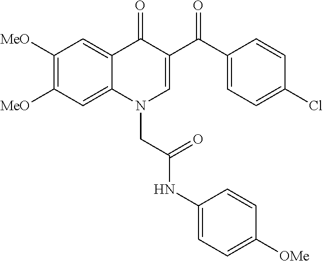 [SMILES: COc1ccc(NC(=O)Cn2cc(C(=O)c3ccc(Cl)cc3)c(=O)c3cc(OC)c(OC)cc32)cc1] N-p-Anisyl-2-[3-(4-chlorobenzoyl)-6,7-dimethoxy-4- oxo-1-quinolinyl]acetamide |
| 105 | 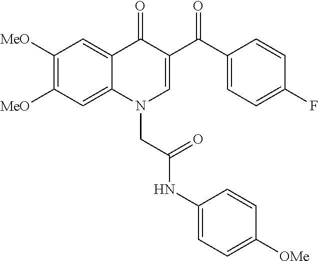 [SMILES: COc1ccc(NC(=O)Cn2cc(C(=O)c3ccc(F)cc3)c(=O)c3cc(OC)c(OC)cc32)cc1] N-p-Anisyl-2-[6,7-dimethoxy-3-(4-fluorobenzoyl)-4- oxo-1-quinolinyl]acetamide |
| 106 | 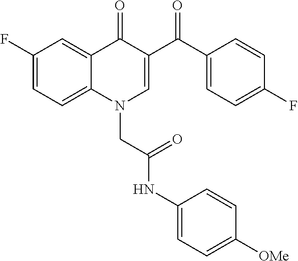 [SMILES: COc1ccc(NC(=O)Cn2cc(C(=O)c3ccc(F)cc3)c(=O)c3cc(F)ccc32)cc1] N-p-Anisyl-2-[6-fluoro-3-(4-fluorobenzoyl)-4-oxo-1- quinolinyl]acetamide |
| 107 | 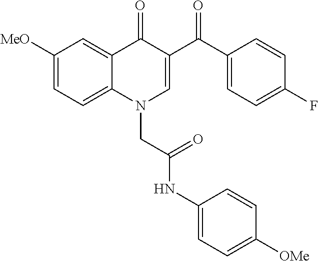 [SMILES: COc1ccc(NC(=O)Cn2cc(C(=O)c3ccc(F)cc3)c(=O)c3cc(OC)ccc32)cc1] N-p-Anisyl-2-[3-(4-fluorobenzoyl)-6-methoxy-4-oxo- 1-quinolinyl]acetamide |
| 108 | 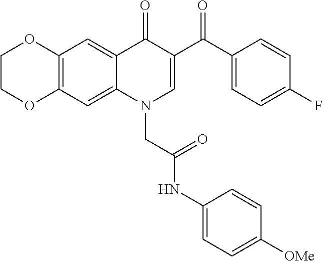 [SMILES: COc1ccc(NC(=O)Cn2cc(C(=O)c3ccc(F)cc3)c(=O)c3cc4c(cc32)OCCO4)cc1] N-p-Anisyl-2-[6,7-ethylenedioxy-3-(4-fluorobenzoyl)- 4-oxo-1-quinolinyl]acetamide |
| 109 | 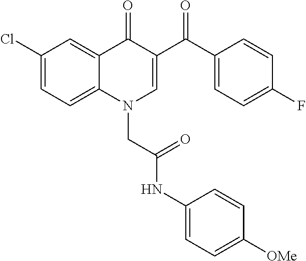 [SMILES: COc1ccc(NC(=O)Cn2cc(C(=O)c3ccc(F)cc3)c(=O)c3cc(Cl)ccc32)cc1] N-p-Anisyl-2-[6-chloro-3-(4-fluorobenzoyl)-4-oxo-1- quinolinyl]acetamide |

### Biological Examples

### Biological Example 1

To cells (HL-60 or PLB-985) in HBSS medium was added compound (25 or 50 μM), isoluminol and Horse Radish Peroxidase, fraction II. Luminescence was measured for 10-30 minutes and the area under the absorbance curve was divided by that from a control experiment to give the activity expressed as fold change.

### Biological Example 2

### Evaluation of the Effect of ROS on Arthritogenic T Cells

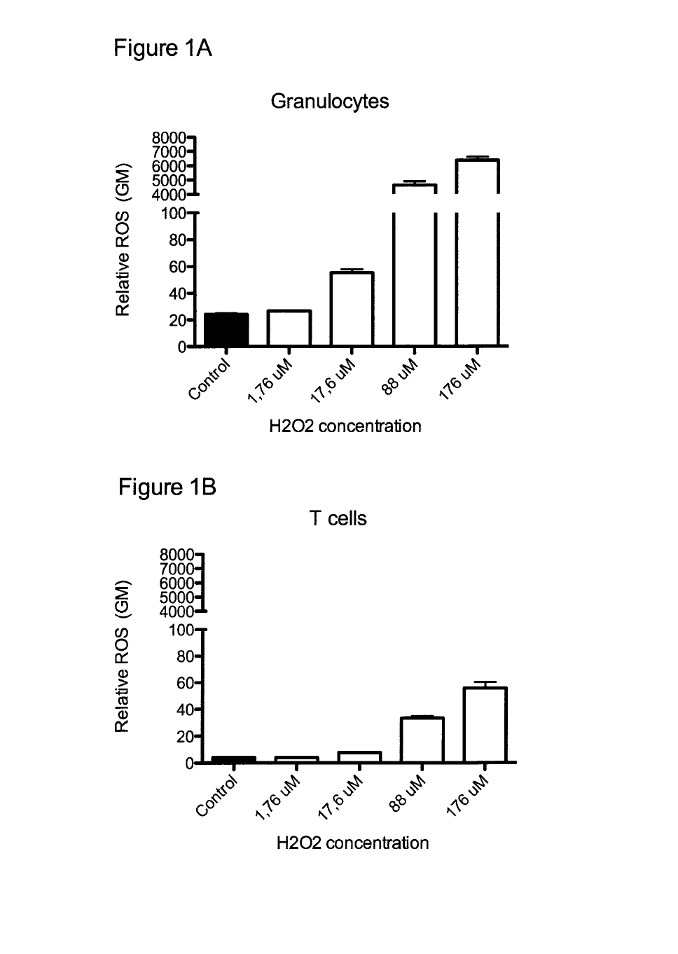

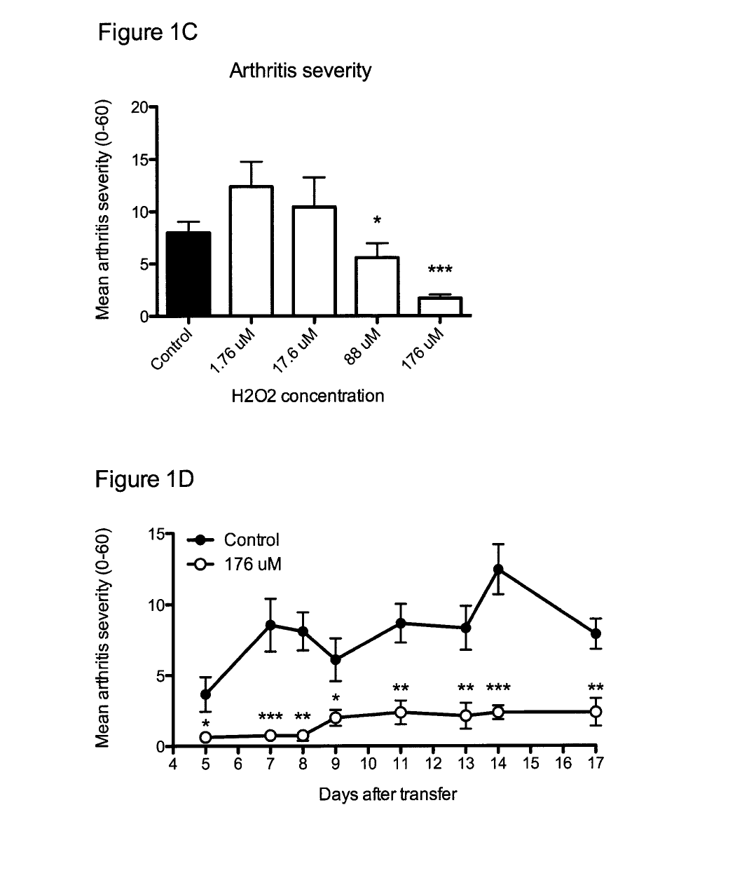

In order to measure the effect of exogenous ROS on intracellular ROS content, single cell suspensions were prepared from rat spleens. After lysis of red blood cells (0.84% ammonium chloride) the cells were stained with antibody His-48-bio (anti-granulocyte) or R73-bio (anti-T cell receptor) followed by staining with streptavidine-APC. Cells were then resuspended in DMEM containing DHR-123 and incubated for 10 minutes at 37° C. Hydrogen peroxide (H₂O₂) was added to a final concentration ranging from 176 μM to 1.76 μM. After 20 minutes incubation, cells were washed, resuspended in PBS and acquired in a FACScalibur. As shown in FIG. 1 a dose dependent relationship could be detected analyzing for the relative ROS content was analysed for A) Granulocytes and B) T cells. Samples were analysed in triplicates.

In order to investigate the effect of ROS on T cell arthritogenicity, spleens were collected from arthritis susceptible DA rats injected with the arthritis inducing oil pristane (0.5 ml per rat s.c.) day −13. Single cell suspensions were prepared from the spleens and red blood cells were lysed. After wash, cells were resuspended in cell culture medium containing concanavalin A (ConA) to a final concentration of 4.5×10̂6 cells/ml. H₂O₂ was added to the culture flasks to a final concentration ranging from 176 μM to 1.76 μM. The cells were incubated for 44 hours at 37° C. After incubation the cells were washed and resuspended in PBS to a final concentration of 60*10̂6 cells/ml. Cells were injected i.p. (1 ml/rat) into naïve recipient DA rats and disease was followed by macroscopic evaluation of disease.

Increased cellular ROS, in this experiment provided as exogenous H₂O₂ significantly alters the T cells capacity to transfer arthritis to naïve recipients in a concentration dependent fashion as shown in FIG. 1 as C) Mean arthritis score during the experiment and D) disease curve where score per day is indicated (n=7-8).

All data is presented as mean±SEM. * represent a p-value <0.05, ** represent a p-value <0.01, *** represent a p-value <0.001.

### Biological Example 3

### Identification of ROS Inducing Substances and Effect on Arthritogenic T Cells

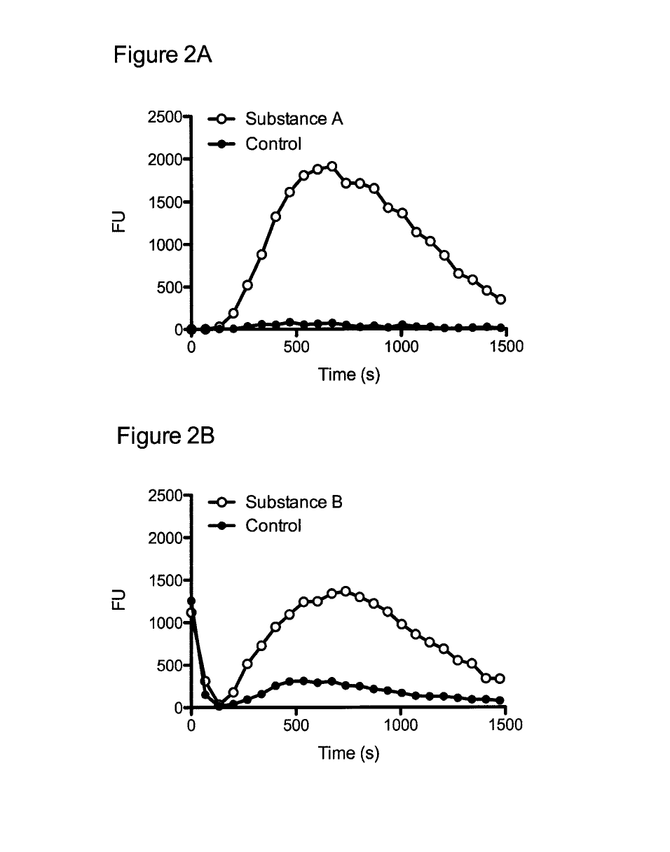

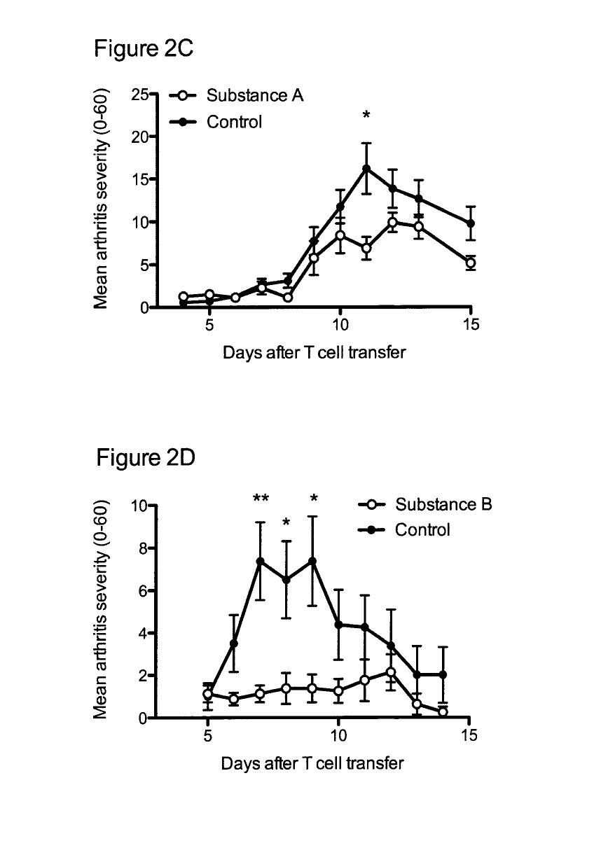

Rat granulocytes were recruited to the peritoneum by an i.p. injection of 2.4% thioglycollate medium (autoclaved). The peritoneal cavity was washed after 24 hours with ice cold HBSS. Collected cells were, after lysis of red blood cells, washed and resupended in HBSS. In a total volume of 100 μl, compound (25 μM), isoluminol (0.175 mg/ml) and horseradish peroxidase (fraction II) (1.75 U/ml) was added to the cells (0.1*10̂6/well) and resulting ROS production detected as a luminescence signal (fluorescent unit, FU) over time. Control cells were treated with vehicle control (DMSO). All samples were analysed at least in duplicates. A clear induction of cellular ROS production could be seen after addition of substance A and B (as defined herein above) compared to control cells, FIGS. 2 A and B.

In order to investigate the effect of ROS inducing substances on T cell arthritogenicity, spleens were collected from arthritis susceptible DA rats injected with the arthritis inducing oil pristane (0.5 ml per rat s.c.) day −13. Single cell suspensions were prepared from the spleens and red blood cells were lysed.

After wash, cells were resuspended in cell culture medium containing concanavalin A (ConA) to a final concentration of 4.5×10̂6 cells/ml. Substance A or B was added to the cells to a final concentration of 12.5 μM. The cells were incubated for 44 hours at 37° C. Control cells were incubated in presence of vehicle control (DMSO). After incubation the cells were washed and resuspended in PBS to a final concentration of C) 30*10̂6 and D) 60*10̂6 cells/ml. Cells were injected i.p. (1 ml/rat) into naïve recipient DA rats and disease was followed by macroscopic evaluation of disease (n=8-11). As shown in FIGS. 2 C and D ROS inducing substances A and B respectively significantly alters the T cells capacity to transfer arthritis to naïve recipients.

All data is presented as mean±SEM. * represent a p-value <0.05, ** represent a p-value <0.01, *** represent a p-value <0.001.

### Biological Example 4

Examples compounds of the invention as described above were tested in the biological in vitro assay described in Biological Example 1 and were found to be stimulators of ROS production. Each assay indicated below was performed at a different site, at a different time, by different personnel and/or using different cell lines, concentrations and controls. The cell line, concentration of example compound and control used in respect of each assay were as indicated below. The values obtained from these different setups (each using an otherwise identical assay) are given as Activity A, B, C, D or E in Table 5 below. The values are expressed as fold increase of NADPH activity in the presence of the relevant of compound (as indicated below) compared to the relevant control (as indicated below).

Activity A: HL-60 cells; 50 μM compound vs. blank
Activity B: PLB-985 cells; 25 μM compound vs. blank
Activity C, D and E: PLB-985 cells; 25 μM compound vs. DMSO

TABLE 5

| Example | Activity A | Activity B | Activity C | Activity D | Activity E |
|---|---|---|---|---|---|
| 1 | | | 45.3 | 27.4 | 8.5 |
| 2 | | | | | 10.0 |
| 3 | | | | | 1.8 |
| 4 | | | | | 3.4 |
| 5 | | | | | 4.3 |
| 6 | | | | | 1.7 |
| 7 | | | | | 2.2 |
| 8 | | | | | 1.3 |
| 9 | | | | | 2.0 |
| 10 | | | | | 1.7 |
| 11 | | | | | 8.8 |
| 12 | | | | | 3.2 |
| 13 | | | | | |
| 14 | | | | | |
| 15 | | | | | 4.3 |
| 16 | | | | 35.4 | 5.9 |
| 17 | | | | 16.3 | |
| 18 | | | | 12.6 | |
| 19 | | | | 10.9 | |
| 20 | | | | 6.3 | |
| 21 | | | | 68.7 | 9.2 |
| 22 | | | | | 20.2 |
| 23 | | | | | 2.1 |
| 24 | | | | | 1.7 |
| 25 | | | | | 34.6 |
| 26 | | | 14.9 | | |
| 28 | | | 16.1 | | |
| 29 | | | 55.0 | | |
| 30 | | | 28.2 | | 4.1 |
| 31 | | | 49.5 | | |
| 32 | | | 47.8 | | |
| 33 | | | 45.6 | | |
| 34 | | | 27.5 | | |
| 35 | | | 6.3 | | |
| 36 | | | 46.4 | | |
| 37 | | | | | 8.4 |
| 38 | | | | | 7.0 |
| 39 | | | | | 76.2 |
| 40 | | | | | 73.0 |
| 41 | | | | | 18.2 |
| 42 | | | | | 2.5 |
| 43 | | | | | 3.8 |
| 44 | | | | | 78.1 |
| 45 | | | 10.7 | | |
| 46 | | 126.8 | 40.7 | | |
| 47 | | 144.2 | 58.4 | | |
| 48 | | 81.4 | 42.5 | | |
| 49 | | 151.7 | 56.4 | | |
| 50 | | 163.2 | 62.8 | | |
| 51 | | 174.2 | 66.7 | | |
| 52 | | 154.4 | 55.6 | | |
| 53 | | 150.1 | | | |
| 54 | | 74.2 | 61.5 | 13.5 | |
| 55 | | 6.3 | 19.6 | | |
| 56 | | 6.4 | 4.4 | | |
| 57 | | 4.4 | 2.3 | | |
| 58 | | 2.5 | 3.6 | | |
| 59 | | 7.6 | 13.4 | | |
| 60 | | 51.8 | 33.3 | | |
| 61 | | 35.9 | 28.5 | | |
| 62 | | 51.0 | 26.1 | | |
| 63 | | 37.3 | 24.3 | | |
| 64 | | 41.1 | 27.9 | | |
| 65 | | 4.1 | 2.8 | | |
| 66 | | 2.7 | 2.5 | | |
| 67 | | 2.3 | 1.8 | | |
| 68 | | 10.1 | 6.7 | | |
| 69 | | 4.6 | 4.0 | | |
| 70 | | 33.2 | 25.4 | | |
| 71 | | 43.6 | 25.1 | | |
| 72 | | 62.6 | 33.6 | | |
| 73 | | 15.2 | 17.1 | | |
| 74 | | 38.0 | 20.1 | | |
| 75 | | 7.7 | 5.3 | | |
| 76 | | 43.6 | 23.9 | | |
| 77 | | | 2.8 | | |
| 78 | | | 11.6 | | |
| 79 | | | 3.2 | | |
| 80 | | | 3.2 | | |
| 81 | | | 22.6 | | |
| 82 | | | 5.9 | | |
| 83 | | | 2.0 | | |
| 84 | | | 8.4 | | |
| 85 | | | 12.2 | | |
| 86 | | 22.7 | 28.3 | | |
| 87 | | | 27.2 | | |
| 88 | | | 31.4 | | |
| 89 | | 33.0 | 34.4 | 17.1 | |
| 90 | | 40.9 | 37.6 | | |
| 91 | 5.3 | | | | |
| 92 | 8.2 | | | | |
| 93 | 2.9 | | | | |
| 94 | 8.4 | | | | |
| 95 | 4.5 | | | | |
| 96 | 2.3 | | | | |
| 97 | 6.0 | | | | |
| 98 | 9.0 | | | | |
| 99 | 4.3 | | | | |
| 100 | 2.9 | | | | |
| 101 | 2.1 | | | | |
| 102 | | | | 2.1 | |
| 103 | | | | 3.7 | |
| 104 | | | | 1.4 | |
| 105 | | | | 2.0 | |
| 106 | | | | 5.6 | |
| 107 | | | | 6.6 | |
| 108 | | | | 1.3 | |
| 109 | | | | 2.5 | |

### REFERENCES

The following documents may be referred to herein, and are hereby incorporated by reference in their entirety:
Bae, Y. S., T. G. Lee, et al. (2003). “Identification of a compound that directly stimulates phospholipase C activity.” Mol Pharmacol 63(5): 1043-50.
Bae, Y. S., J. Y. Song, et al. (2003). “Differential activation of formyl peptide receptor signaling by peptide ligands.” Mol Pharmacol 64(4): 841-7.
Bjorkman, L., C. Dahlgren, et al. (2008). “Phagocyte-derived reactive oxygen species as suppressors of inflammatory disease.” Arthritis Rheum 58(10): 2931-5.
Bylund, J., M. Samuelsson, et al. (2003). “NADPH-oxidase activation in murine neutrophils via formyl peptide receptors.” Exp Cell Res 282(2): 70-7.
Christophe, T., A. Karlsson, et al. (2001). “The synthetic peptide Trp-Lys-Tyr-Met-Val-Met-NH2 specifically activates neutrophils through FPRL1/lipoxin A4 receptors and is an agonist for the orphan monocyte-expressed chemoattractant receptor FPRL2.” J Biol Chem 276(24): 21585-93.
De Ravin, S. S., N. Naumann, et al. (2008). “Chronic granulomatous disease as a risk factor for autoimmune disease.” J Allergy Clin Immunol 122(6): 1097-103.
Forsman, H., C. Kalderen, et al. “Stable formyl peptide receptor agonists that activate the neutrophil NADPH-oxidase identified through screening of a compound library.” Biochem Pharmacol 81(3): 402-11.
Greve, B., P. Hoffmann, et al. (2008). “NCF1 gene and pseudogene pattern: association with parasitic infection and autoimmunity.” Malar J 7(1): 251.
Hultqvist, M., P. Olofsson, et al. (2006). “A new arthritis therapy with oxidative burst inducers.” PLoS Med 3(9): e348.
Hultqvist, M., P. Olofsson, et al. (2004). “Enhanced autoimmunity, arthritis, and encephalomyelitis in mice with a reduced oxidative burst due to a mutation in the Ncf1 gene.” Proc Natl Acad Sci USA 101(34): 12646-51.
Jaquet, V., L. Scapozza, et al. (2009). “Small-molecule NOX inhibitors: ROS-generating NADPH oxidases as therapeutic targets.” Antioxid Redox Signal 11(10): 2535-52.
Kirpotina, L. N., A. I. Khlebnikov, et al. “Identification of novel small-molecule agonists for human formyl peptide receptors and pharmacophore models of their recognition.” Mol Pharmacol 77(2): 159-70.
Krjukova, J., T. Holmqvist, et al. (2004). “Phospholipase C activator m-3M3FBS affects Ca2+ homeostasis independently of phospholipase C activation.” Br J Pharmacol 143(1): 3-7.
Lambeth, J. D., K. H. Krause, et al. (2008). “NOX enzymes as novel targets for drug development.” Semin Immunopathol 30(3): 339-63.
Mossberg, N., O. Andersen, et al. (2007). “Oxygen radical production and severity of the Guillain-Barre syndrome.” J Neuroimmunol 192(1-2): 186-91.
Mossberg, N., C. Movitz, et al. (2009). “Oxygen radical production in leukocytes and disease severity in multiple sclerosis.” J Neuroimmunol 213(1-2): 131-4.
Olofsson, P., J. Holmberg, et al. (2003). “Positional identification of Ncf1 as a gene that regulates arthritis severity in rats.” Nat Genet. 33(1): 25-32.
Olsson, L. M., A. K. Lindqvist, et al. (2007). “A case-control study of Rheumatoid arthritis identifies an associated SNP in the NCF4 gene supporting a role for the NOX-complex in autoimmunity.” Arthritis Res Ther 9(5): R98.
Rabiet, M. J., E. Huet, et al. (2005). “Human mitochondria-derived N-formylated peptides are novel agonists equally active on FPR and FPRL1, while Listeria monocytogenes-derived peptides preferentially activate FPR.” Eur J Immunol 35(8): 2486-95.
Rioux, J. D., R. J. Xavier, et al. (2007). “Genome-wide association study identifies new susceptibility loci for Crohn disease and implicates autophagy in disease pathogenesis.” Nat Genet. 39(5): 596-604.
Roberts, R. L., J. E. Hollis-Moffatt, et al. (2008). “Confirmation of association of IRGM and NCF4 with ileal Crohn's disease in a population-based cohort.” Genes Immun 9(6): 561-5.
Schepetkin, I. A., L. N. Kirpotina, et al. (2007). “High-throughput screening for small-molecule activators of neutrophils: identification of novel N-formyl peptide receptor agonists.” Mol Pharmacol 71(4): 1061-74.
Schepetkin, I. A., L. N. Kirpotina, et al. (2008). “Identification of novel formyl peptide receptor-like 1 agonists that induce macrophage tumor necrosis factor alpha production.” Mol Pharmacol 74(2): 392-402.
Wan, H. X., C. Zhou, et al. (2007). “Discovery of Trp-Nle-Tyr-Met as a novel agonist for human formyl peptide receptor-like 1.” Biochem Pharmacol 74(2): 317-26.
Yu, B., Y. Chen, et al. “The association between single-nucleotide polymorphisms of NCF2 and systemic lupus erythematosus in Chinese mainland population.” Clin Rheumatol.
Zhou, L. and D. Y. Zhu (2009). “Neuronal nitric oxide synthase: structure, subcellular localization, regulation, and clinical implications.” Nitric Oxide 20(4): 223-30.

1. A compound of formula I, 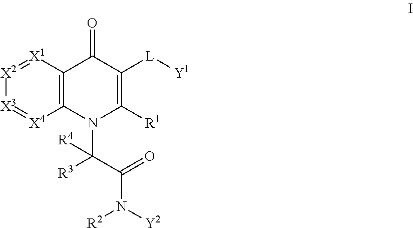
or a pharmaceutically acceptable salt thereof, for use in the treatment, prevention and/or prophylaxis of an autoimmune disease and/or an inflammatory disease,
wherein in respect of the compound of formula I:
X¹, X², X³ and X⁴ each represents CR⁵, CR⁶, CR⁷, CR⁸ respectively or, any one or two of X¹ to X⁴, alternatively and independently represents N;
R⁵, R⁶, R⁷ and R⁸ each independently represents hydrogen, halogen, —R^{a}, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, OS(O)₂R^{p}, —S(O)ₘR^{q}, —S(O)₂N(R^{r})R^{s}, or —SR^{t}; or
any two R⁵, R⁶, R⁷ or R⁸ groups, when adjacent to one another, may be linked by a —C₁₋₅alkylene-, —O—C₁₋₄alkylene-, —C₁₋₄alkylene-O—, —O—C₁₋₃alkylene-O—, —S—C₁₋₄alkylene-, —C₁₋₄alkylene-S— or —S—C₁₋₃alkylene-S— group, which alkylene moieties are optionally substituted by one or more substituents selected from —F, C₁₋₃alkyl, optionally substituted by one or more —F, and ═O;
but characterized in that:
at least one of R⁵ to R⁸ is present and represents —ORⁿ, —SR^{t} or halogen, or two adjacent groups selected from R⁵ to R⁸ are both present and are linked together to form a —O—C₁₋₄alkylene-, —C₁₋₄alkylene-O—, —O—C₁₋₃alkylene-O—, —S—C₁₋₄alkylene-, —C₁₋₄alkylene-S— or —S—C₁₋₃alkylene-S— group;
L represents —C(O)— or —S(O)₂—;
Y¹ represents aryl or heteroaryl, both of which are optionally substituted by one or more substituents selected from E¹;
Y² represents aryl or heteroaryl, both of which are optionally substituted by one or more substituents selected from E²;
E¹ and E² each independently represent halogen, —R^{a}, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, —OS(O)₂R^{p}, —S(O)ₘR^{q}, —S(O)₂N(R^{r})R^{s}, or —SR^{t}; and/or
any two E¹ or E² substituents, when adjacent to one another, may be linked to form a —C₁₋₅alkylene-, —O—C₁₋₄alkylene-, —C₁₋₄alkylene-O—, —O—C₁₋₃alkylene-O—, —S—C₁₋₄alkylene-, —C₁₋₄alkylene-S— or —S—C₁₋₃alkylene-S— group, which alkylene moieties are optionally substituted by one or more substituents selected from —F, C₁₋₃alkyl, optionally substituted by one or more —F, and ═O;
R¹ and R² each independently represent hydrogen or C₁₋₆ alkyl, optionally substituted by one or more substituents selected from —F, —OMe, —OEt, —OCHF₂ and —OCF₃;
R³ and R⁴ each independently represent hydrogen or C₁₋₆ alkyl, optionally substituted by one or more substituents selected from —F, —OMe, —OEt, —OCHF₂ and —OCF₃; or
R³ and R⁴ may be linked to form a further 3- to 6-membered ring, which may optionally contain one or more heteroatoms and may optionally be further substituted by one or more substituents selected from C₁₋₆alkyl, optionally substituted by one or more —F, and ═O;
each R^{a}, R^{b}, R^{c}, R^{d}, R^{e}, R^{f}, R^{g}, R^{h}, Rⁱ, R^{j}, R^{l}, Rⁿ, R^{o}, R^{q}, R^{r}, R^{s} and R^{t} independently represents hydrogen or C₁₋₆ alkyl, optionally substituted by one or more —F; or
any two R^{c} and R^{d}, R^{f} and R^{g} and/or R^{r} and R^{s} may be linked together with the nitrogen atom to which they are necessarily attached, to form a 3- to 8-membered monocyclic or bicyclic ring, which ring may optionally contain one or two heteroatoms, and which ring may optionally be substituted by one or more substituents selected from —F, C₁₋₃alkyl, optionally substituted by one or more —F, and ═O;
each R^{k}, R^{m} and R^{p} independently represent C₁₋₆ alkyl, optionally substituted by one or more —F;
m represents 0, 1 or 2.

2. A compound as claimed in claim 1, wherein:
at least one of R⁶ and R⁷ is present and represents —ORⁿ, or
R⁶ and R⁷ are both present and are linked together to form a —O—C₁₋₄alkylene-, —C₁₋₄alkylene-O— or —O—C₁₋₃ alkylene-O— group.

3. A compound as claimed in claim 2, wherein:
R⁶ and R⁷ are both present and are linked together to form a —O—C₁₋₃ alkylene-O— group.

4. A compound as claimed in claim 1, wherein:
X¹, X², X³ and X⁴ each represents CR⁵, CR⁶, CR⁷, CR⁸ respectively or any one of X¹ to X⁴, alternatively and independently represents N;
E¹ and E² each independently represent halogen, —R^{a}, —CN, C(O)N(R^{c})R^{d}, —N(R^{f})R^{g}, —N(R^{h})C(O)Rⁱ, —N(R^{j})C(O)OR^{k}, NO₂, —N(R^{l})S(O)₂—R^{m}, —ORⁿ, —S(O)ₘR^{q} or S(O)₂N(R^{r})R^{s}; or
any two E¹ or E² substituents, when adjacent to one another, may be linked to form a —C₁₋₅alkylene-, O—C₁₋₄alkylene-, —C₁₋₄alkylene-O— or a O—C₁₋₃alkylene-O— group, which groups are optionally substituted by one or more substituent selected from —F, C₁₋₃alkyl, optionally substituted by one or more —F, and ═O;
R¹ represents hydrogen, -Me, -Et, —CH₂F, —CHF₂ or —CF₃;
R² represents hydrogen, -Me or -Et;
R³ and R⁴ each independently represents hydrogen or -Me, or R³ and R⁴ may be linked to form a further 3- to 4-membered ring;
R⁵, R⁶, R⁷ and R⁸ each independently represents hydrogen, halogen, —R^{a}, —CN, C(O)N(R^{c})R^{d}, —N(R^{f})R^{g}, —N(R^{h})C(O)Rⁱ, —N(R^{j})C(O)OR^{k}, NO₂, —N(R^{l})S(O)₂—R^{m}, —ORⁿ, —S(O)ₘR^{q}, —S(O)₂N(R^{r})R^{s} or —SR^{t}; or
any two R⁵, R⁶, R⁷ or R⁸ groups, when adjacent to one another, may be linked by a —C₁₋₅alkylene-, O—C₁₋₄alkylene-, —C₁₋₄alkylene-O— or a O—C₁₋₃alkylene-O— group, which groups are optionally substituted by one or more —F or -Me; and/or
each R^{a}, R^{c}, R^{d}, R^{f}, R^{g}, R^{h}, Rⁱ, R^{j}, R^{l}, Rⁿ, R^{q}, R^{r}, R^{s} and R^{t} independently represents hydrogen or C₁₋₃ alkyl, optionally substituted by one, two or three —F; or
any two R^{c} and R^{d}, R^{f} and R^{g} and/or R^{r} and R^{s} may be linked together with the nitrogen atom to which they are necessarily attached, to form a 4- to 6-membered monocyclic ring, which ring may optionally contain one additional heteroatom, and which ring may optionally be substituted by C₁₋₃alkyl, optionally substituted by one, two or three —F.

5. A compound as claimed in claim 1, but in which either:
(a) R⁶ and R⁷ independently represent hydrogen, halogen, C₂₋₆ alkyl, optionally substituted by one or more fluorine atoms, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, OS(O)₂R^{p}, —S(O)ₘR^{q}, —S(O)₂N(R^{r})R^{s}, or —SR^{t}; or
(b) R⁶ and R⁷ are both present and independently represent halogen, C₁₋₆ alkyl, optionally substituted by one or more fluorine atoms, —CN, —C(O)R^{b}, —C(O)N(R^{c})R^{d}, —C(O)OR^{e}, —N(R^{f})R^{g}, —N(R^{h})C(O)Rⁱ, —N(R^{j})C(O)OR^{k}, —NO₂, —N(R^{l})S(O)₂—R^{m}, —ORⁿ, —OC(O)R^{o}, OS(O)₂R^{p}, —S(O)ₘR^{q}, —S(O)₂N(R^{r})R^{s}, or —SR^{t}.

6. A compound as claimed in claim 5, wherein either:
(a) R⁶ and R⁷ independently represent hydrogen, halogen or C₂₋₆ alkyl, optionally substituted by one or more fluorine atoms; or
(b) R⁶ and R⁷ are both present and independently represent halogen or C₁₋₆ alkyl, optionally substituted by one or more fluorine atoms.

7. A method of treatment of a subject with a disease that would benefit from increased NADPH oxidase activity and/or increased production of reactive oxygen species, by administering to the subject a therapeutically effective amount of a compound of formula I as defined in claim 1.

8. The method of claim 7, wherein the disease is an autoimmune disease selected from rheumatoid arthritis, multiple sclerosis, inflammatory bowel disease, Crohn's disease, and ulcerative colitis, systemic lupus erythematosus, autoimmune uveitis, type I diabetes, dermatomyesitis, Goodpasteure's syndrome, Graves' disease, Guillian-Barré Syndrome (GBS), Hashimotos Disease, Mixed connective tissue disease, Myasthenia gravis, Pemphigus vulgaris, Pernicious anemia, Psoriasis, Polymyositis, Primary biliary cirrhosis, Sjögren's syndrome, Giant cell arteritis, Ulcerative colitis, Vasculitis, Wegener's granulomatosis, Churg-Strauss syndrome and iopathic thrombocytopenic purpura, or the disease is an inflammatory disease selected from celiac disease, vasculitis, lupus, chronic obstructive pulmonary disease (COPD), irritable bowel disease, atherosclerosis, arthritis and psoriasis.

9. A pharmaceutical composition, comprising a compound is of formula I as defined in claim 1, or a pharmaceutically acceptable salt thereof, but in which:
(i) when L represents —C(O)—;
R², R³ and R⁴ each represent hydrogen;
Y² represents 4-fluorophenyl;
X¹, X², X³ and X⁴ respectively represent CR⁵, CR⁶, CR⁷ and CR⁸,
R⁵, R⁷ and R⁸ each represent hydrogen;
R⁶ represents —OCH₃,
then Y¹ does not represent phenyl substituted at the 4-position with chlorine, fluorine or methyl;
(ii) when L represents —S(O)₂—;
R², R³ and R⁴ each represent hydrogen;
Y² represents phenyl;
X¹, X², X³ and X⁴ respectively represent CR⁵, CR⁶, CR⁷ and CR⁸,
R⁵, R⁷ and R⁸ each represent hydrogen;
R⁶ represents fluorine,
then Y¹ does not represent phenyl substituted at the 4-position with methyl;
(iii) when L represents —S(O)₂—;
R², R³ and R⁴ each represent hydrogen;
Y² represents 4-methylphenyl;
X¹, X², X³ and X⁴ respectively represent CR⁵, CR⁶, CR⁷ and CR⁸,
R⁵, R⁷ and R⁸ each represent hydrogen;
R⁶ represents fluorine,
then Y¹ does not represent phenyl, or phenyl substituted at the 4-position with methyl or fluorine; and
a pharmaceutically acceptable adjuvant, diluent or carrier.

10. (canceled)

11. A method of treating a subject with a disease that would benefit from increased NADPH oxidase activity and/or increased production of a reactive oxygen species, comprising administering to the subject a pharmaceutical formulation as claimed in claim 9.

12. (canceled)

13. (canceled)

14. A combination product comprising:
(A) a compound of formula I as defined in claim 1, or a pharmaceutically-acceptable salt thereof; and
(B) one or more therapeutic agent(s) that is/are useful in the treatment of a disease selected from an autoimmune disease, an inflammatory disease or another disease that would benefit from increased NADPH oxidase activity and/or increased production of reactive oxygen species,
wherein each of components (A) and (B) is formulated in admixture with a pharmaceutically-acceptable adjuvant, diluent or carrier.

15. A process for the preparation of a pharmaceutical formulation, which process comprises bringing into association a compound of formula I, as defined in claim 1, or a pharmaceutically acceptable salt thereof with a pharmaceutically-acceptable adjuvant, diluent or carrier.

16. A process for the preparation of a combination product as defined in claim 14, which process comprises bringing into association a compound of formula I, or a pharmaceutically acceptable salt thereof with the other therapeutic agent(s) that is/are useful in the treatment of a cancer, and at least one pharmaceutically-acceptable adjuvant, diluent or carrier.

17. A method for identifying a compound as defined in claim 1, as being potentially suitable for combined ROS activation and T cell regulation, or, a compound as defined in claim 1, which combines ROS activation and T cell regulation, comprising:
incubating a test agent and NADPH oxidase sufficient cells and measuring ROS production; and
comparing the ROS production of the same cells in absence of test agent.
